(19) United States
(12) Patent Application Publication
(10) Pub. No.: US 2005/0203081 A1
(43) Pub. Date: Sep. 15, 2005

(54) Inhibitors of protein tyrosine phosphatase 1B

(76) Inventors: Jinbo Lee, Andover, MA (US); Steve J. Kirincich, Concord, MA (US); Michael J. Smith, Poland, OH (US); Douglas P. Wilson, Ayer, MA (US); Bruce C. Follows, Littleton, MA (US); Zhao-Kui Wan, Arlington, MA (US); Diane M. Joseph-McCarthy, Belmont, MA (US); David V. Erbe, Somerville, MA (US); Yan-Ling Zhang, Lexington, MA (US); Weixin Xu, Acton, MA (US); Steve Y. Tam, Wellesley, MA (US)

Correspondence Address: FISH & RICHARDSON P.C., 225 FRANKLIN STREET, BOSTON, MA, 02110, US

(21) Appl. No.: 11/064,390

(22) Filed: Feb. 23, 2005

Related U.S. Application Data
(60) Provisional application No. 60/547,071, filed on Feb. 25, 2004.

(51) Int. Cl. A61K 31/55; A61K 31/4743; A61K 31/407; A61K 31/381; C07D 498/02

(52) U.S. Cl. 514/215; 514/412; 514/443; 514/301; 549/49; 548/453; 546/114

(57) ABSTRACT

Protein tyrosine phosphatases (PTPases) such as PTP1B can play a role in regulating a wide variety of cellular responses such as insulin signaling. Substituted bicyclic fused-thiophene compounds can inhibit PTP1B and thereby induce greater insulin sensitivity. Accordingly, PTP1B inhibition can provide an alternate treatment for PTPase-mediated disorders such as diabetes.

### CROSS REFERENCE TO RELATED APPLICATIONS

This application claims the benefit of U.S. Provisional Application No. 60/547,071 filed on Feb. 25, 2004, which is incorporated herein by reference in its entirety.

### TECHNICAL FIELD

This invention relates to inhibitors of protein tyrosine phosphatase 1B (PTP1B) and other protein tyrosine phosphatases (PTPases).

### BACKGROUND

Protein tyrosine phosphatases (PTPases) are a large family of diverse molecules that can play an important role in modulating a wide variety of cellular responses. The PTPase family is divided into three major subclasses, classical PTPases, low molecular weight PTPases, and dual specificity PTPases. The classical PTPases can be further categorized into two classes, intracellular PTPases (e.g., PTP1B, TC-PTP, rat-brain PTPase, STEP, PTPMEG 1, PTPH1, PTPD1, PTPD2, FAP-1/BAS, PTP1C/SH-PTP1/SHP-1 and PTP1D/Syp/SH-PTP2/SHP2) and receptor-type PTPases (e.g., CD45, LAR, PTPA, PTPP, PTPδ, PTPε, PTPγ, SAP-1 and DEP-1). Dual specificity phosphatases have the ability to remove the phosphate group from both serine/threonine and tyrosine residues. Members of the PTPase family have been implicated as important modulators or regulators of a wide variety of cellular processes including insulin signaling, leptin signaling, T-cell activation and T-cell mediated signaling cascade, the growth of fibroblasts, platelet aggregation, and regulation of osteoblast proliferation.

### SUMMARY

In general, compounds of formula (I), including pharmaceutically acceptable salts or pro-drugs of those compounds, inhibit PTP1B. Pharmaceutical compositions can include one or more compounds of formula (I) or pharmaceutically acceptable salts, or prodrugs of those or more compounds of formula (I) and a pharmaceutically acceptable carrier or excipient. In addition, PTPase-mediated disorders can be treated with and PTPases can be inhibited with compounds of formula (I) or pharmaceutically acceptable salts, or pro-drugs of those compounds.

In one aspect, this invention features compounds of formula (I):

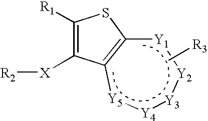

R₁ is C(O)OR₇, 5- to 6-membered heterocycle, H, halogen, CN, or C(O)NR₇R₈.

R₂ is C(O)ZR₄ or CN.

Z is —O— or —NR₅—.

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene and alkynylene groups can be optionally substituted with one or more halogen, oxo, HN═, CN, OCF₃, OH, NH₂, NO₂, R₄, or Q.

Each Y₁, Y₂, Y₃, Y₄, and Y₅ is, independently, CR₃, N, S, or O. One or two of Y₁, Y₂, Y₃, Y₄, and Y₅ can be absent.

Each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q. Any of the aryl, heterocyclic, alkyl, alkenyl or alkynyl groups is optionally substituted with one or more halogen, oxo, CN, OCF₃, OH, NH₂, NO₂, N₃, R₄, or Q.

Each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅.

Each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl. Each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈.

Each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl. Each R₇, R₈, and R₉ can be optionally substituted with one or more halogen, oxo, CN, OCF₃, OH, NH₂, or NO₂.

When R₃ is H, the ring system is 1-benzothiophene, R₁ is C(O)OCH₃, and X is —OCH₂—, then R₂ is not C(O)OCH₃.

When R₃ is H, the ring system is 1-benzothiophene, R₁ is C(O)OH, and X is —OCH₂—, then R₂ is not C(O)OH.

When R₃ is H, the ring system is thieno[2,3-b]pyridine, R₁ is isopropyl ester, and X is —OCH₂—, then R₂ is not C₁₋₃alkyl ester.

When R₃ is H, the ring system is thieno[2,3-b]pyridine, R₁ is C(O)OC₁₋₄alkyl, and X is —OCH₂— or —OCH(CH₃)—, then R₂ is not CN.

When R₃ is H, the ring system is thieno[2,3-b]pyridine, R₁ is isopropyl ester, and X is —SCH₂CH₂—, then R₂ is not CN.

When R₃ is H, the ring system is thieno[2,3-b]pyridine, R₁ is isopropyl ester, and X is —SCH₂—, then R₂ is not isopropyl ester.

The compound of formula (I) can be a salt.

In another aspect, this invention features compounds of formula (I),

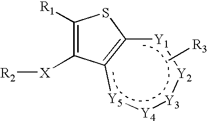

wherein R₁ is C(O)OR₇, 5- to 6-membered heterocycle, H, halogen, CN, or C(O)NR₇R₈;

R₂ is C(O)ZR₄ or CN;

Z is —O— or —NR₅—;

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;

each Y₁, Y₂, Y₃, and Y₄, is, independently, CR₃, N, S, or O; where Y₅ is absent;

each R₃ is, independently, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;

each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;

each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;

each R₇, R₈, and R₉ is, independently, H, C₁₋₁₂alkyl, C₂₋₁₂alkenyl, C₁₋₁₂alkynyl, C₃₋₁₂cycloalkyl, aryl, or arylC₁₋₁₂alkyl; where each R₇, R₈, and R₉ is optionally substituted with one or more halogen, oxo, CN, OCF₃, OH, NH₂, or NO₂.

The compound of formula (I) can be a salt.

In a further aspect, this invention features compounds of formula (I),

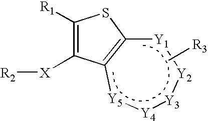

wherein R₁ is C(O)OC₁₋₁₂alkyl, 5- to 6-membered heterocycle, H, halogen, CN, or C(O)NR₇R₈;

R₂ is C(O)ZR₄ or CN, wherein R₄ is not methyl;

Z is —O— or —NR₅—;

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;

each Y₁, Y₂, Y₃, and Y₄, is, independently, CR₃; where Y₅ is absent;

each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;

each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —NR₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;

each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₉R₉, —NR₇C(O)OR₉, —NR₇S(O)₂R₈, —SR₇, —S(O)R₇, —S(O)₂R₇, or —S(O)₂NR₇R₈;

each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₂₋₁₂alkyl; where each R₇, R₈, and R₉ is optionally substituted with one or more halogen, oxo, CN, OCF₃, OH, NH₂, or NO₂.

The compound of formula (I) can be a salt.

In one aspect, this invention features compounds of formula (I),

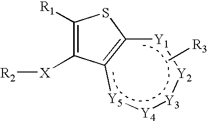

wherein R₁ is C(O)OH, 5- to 6-membered heterocycle, H, halogen, CN, or C(O)NR₇R₈;

R₂ is C(O)ZR₄ or CN, where R₄ is not H;

Z is —O— or —NR₅—;

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;

each Y₁, Y₂, Y₃, and Y₄, is, independently, CR₃; where Y₅ is absent;

each R₃ is, independently, H, aryl, 5- to 8-membered heterocyclyl, C₁₋₆alkyl, C₂₋₆ alkenyl, C₂₋₆ alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;

each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;

each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;

each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₂alkyl; where each R₇, R₈, and R₉ is optionally substituted with one or more halogen, oxo, CN, OCF₃, OH, NH₂, or NO₂.

The compound of formula (I) can be a salt.

In another aspect, this invention features compounds of formula (I),

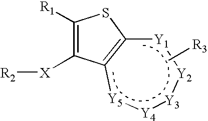

wherein R₁ is C(O)OH, C(O)OC₁₋₂alkyl, C(O)OC₄₋₁₂ alkyl, 5- to 6-membered heterocycle, H, halogen, CN, or C(O)NR₇R₈;

R₂ is C(O)ZR₄;

Z is —O— or —NR₅—;

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;

each Y₁, Y₂, Y₃, and Y₄ is, independently, CR₃, N, S, or O; where Y₅ is absent, and where at least one Y₁, Y₂, Y₃, and Y₄ is N;

each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;

each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NS(O)₂R₅, —SR₄, —S(O)R₄, —S(O)₂R₄, or —S(O)₂NR₄R₅;

each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;

each R₇, R₈, and R₉ is, independently, H, C₁₋₁₂alkyl, C₁₋₁₂alkenyl, C₂₋₁₂alkynyl, C₃₋₁₂cycloalkyl, aryl, or arylC-₁₋₁₂alkyl; where each R₇, R₈, and R₉ is optionally substituted with one or more halogen, oxo, CN, OCF₃, OH, NH₂, or NO₂.

The compound of formula (I) can be a salt.

In a further aspect, this invention relates to compounds of formula (I),

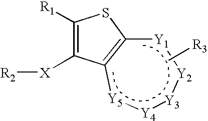

wherein R₁ is C(O)OH, C(O)OC₅₋₁₂alkyl, 5- to 6-membered heterocycle, H, halogen, CN, or C(O)NR₇R₈;

R₂ is C(O)ZR₄ or CN;

Z is —O— or —NR₅—;

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;

each Y₁, Y₂, Y₃, Y₄, and Y₅ is, independently, CR₃, N, S, or O; where one or two of Y₁, Y₂, Y₃, Y₄, and Y₅ can be absent;

each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;

each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;

each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;

each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl; where each R₇, R₈, and R₉ is optionally substituted with one or more halogen, oxo, CN, OCF₃, OH, NH₂, or NO₂.

The compound of formula (I) can be a salt.

In one aspect, a pharmaceutical composition includes at least one of the compounds of formula (I), or a pharmaceutically acceptable salt or prodrug thereof, and a pharmaceutically acceptable excipient or carrier. The compound can inhibit a PTPase such as PTP1B.

For the pharmaceutical composition, the compound of formula (I) can have the following structure:

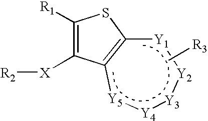

R₁ is C(O)OR₇, 5- to 6-membered heterocycle, H, halogen, CN, or C(O)NR₇R₈.

R₂ is C(O)ZR₄ or CN.

Z is —O— or —NR₅—.

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene and alkynylene groups can be optionally substituted with one or more halogen, oxo, HN═, CN, OCF₃, OH, NH₂, NO₂, R₄, or Q.

Each Y₁, Y₂, Y₃, Y₄, and Y₅ is, independently, CR₃, N, S, or O. One or two of Y₁, Y₂, Y₃, Y₄, and Y₅ can be absent.

Each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q. Any of the aryl, heterocyclic, alkyl, alkenyl or alkynyl groups is optionally substituted with one or more halogen, oxo, CN, OCF₃, OH, NH₂, NO₂, N₃, R₄, or Q.

Each Q is, independently, —OC(O)NR₄R₅, —OR₄, —OC(O)R₄, —COOR₄, —C(O)NR₄R₅, —C(O)R₄, —C(═N—OH)R₄, —NR₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅.

Each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl. Each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₉, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈.

Each R₇, R₈, and R₉ is, independently, H, C₂₋₁₂alkyl, C₂₋₁₂alkenyl, C₂₋₁₂alkynyl, C₃₋₁₂cycloalkyl, aryl, or arylC₁₋₁₂alkyl. Each R₇, R₈, and R₉ can be optionally substituted with one or more halogen, oxo, CN, OCF₃, OH, NH₂, or NO₂.

In one aspect, a method of treating a PTPase-mediated disorder or condition (e.g., a PTP1B-mediated disorder or condition) includes administering to a mammal (e.g., a human) a therapeutically effective amount of a substituted fused, bicyclic thiophene or a pharmaceutically acceptable salt or prodrug thereof.

In another aspect, a method of treating a PTPase-mediated disorder or condition (e.g., a PTP1B-mediated disorder or condition) includes administering to a mammal (e.g., a human) a therapeutically effective amount of a compound of formula (I). In the method of treatment, the compound of formula (I) can have the following structure:

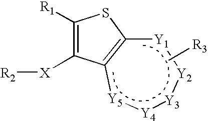

R₁ is C(O)OR₇, 5- to 6-membered heterocycle, H, halogen, CN, or C(O)NR₇R₈.

R₂ is C(O)ZR₄ or CN.

Z is —O— or —NR₅—.

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene and alkynylene groups can be optionally substituted with one or more halogen, oxo, HN═, CN, OCF₃, OH, NH₂, NO₂, R₄, or Q.

Each Y₁, Y₂, Y₃, Y₄, and Y₅ is, independently, CR₃, N, S, or O. One or two of Y₁, Y₂, Y₃, Y₄, and Y₅ can be absent.

Each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q. Any of the aryl, heterocyclic, alkyl, alkenyl or alkynyl groups is optionally substituted with one or more halogen, oxo, CN, OCF₃, OH, NH₂, NO₂, N₃, R₄, or Q.

Each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NS(O)₂R₅, —SR₄, —S(O)R₄, —S(O)₂R₄, or —S(O)₂NR₄R₅.

Each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl. Each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈.

Each R₇, R₈, and R₉ is, independently, H, C₂₋₁₂alkyl, C₂₋₁₂alkenyl, C₂₋₁₂alkynyl, C₃₋₁₂cycloalkyl, aryl, or arylC₁₋₁₂alkyl. Each R₇, R₈, and R₉ can be optionally substituted with one or more halogen, oxo, CN, OCF₃, OH, NH₂, or NO₂.

In one aspect, a method of inhibiting a PTPase activity (e.g., a PTP1B activity) in a sample includes contacting the sample with an effective amount of a substituted fused, bicyclic thiophene or a pharmaceutically acceptable salt or prodrug thereof.

In another aspect, a method of inhibiting a PTPase, such as PTP1B, includes contacting a sample with an effective amount of a compound of formula (I). In the method of inhibiting PTPase, the compound of formula (I) can have the following structure:

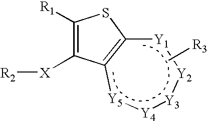

R₁ is C(O)OR₇, 5- to 6-membered heterocycle, H, halogen, CN, or C(O)NR₇R₈.

R₂ is C(O)ZR₄ or CN.

Z is —O— or —NR₅—.

X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, or —C₂₋₄alkynylene-. Any of the alkylene, alkenylene and alkynylene groups can be optionally substituted with one or more halogen, oxo, HN═, CN, OCF₃, OH, NH₂, NO₂, R₄, or Q.

Each Y₁, Y₂, Y₃, Y₄, and Y₅ is, independently, CR₃, N, S, or O. One or two of Y₁, Y₂, Y₃, Y₄, and Y₅ can be absent.

Each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q. Any of the aryl, heterocyclic, alkyl, alkenyl or alkynyl groups is optionally substituted with one or more halogen, oxo, CN, OCF₃, OH, NH₂, NO₂, N₃, R₄, or Q.

Each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅.

Each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl. Each R₄, R₅, and R₆ can be optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈.

Each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl. Each R₇, R₈, and R₉ can be optionally substituted with one or more halogen, oxo, CN, OCF₃, OH, NH₂, or NO₂.

In another aspect, the present invention relates to methods for testing PTP1B inhibitors.

Embodiments can include one or more of the following features.

R₁ can be C(O)OH.

R₁ can be C(O)OCH₃.

R₁ can be C(O)NH₂.

R₁ can be C(O)NHCH₃.

R₁ can be CN.

R₁ can be a 5-membered heterocycle.

X can be —O—C₁₋₃alkylene-(e.g., —OCH₂—, —OCHF—).

R₂ can be C(O)OH.

R₂ can be C(O)OCH₃.

R₂ can be C(O)OC₂₋₄alkane.

X can be —OCH₂— and R₂ can be C(O)OH.

R₂ can be C(O)NH₂.

R₂ can be CN.

Y₅ can be absent and each Y₁, Y₂, Y₃, and Y₄ can be CR₃.

Y₅ can be absent and where one of Y₁, Y₂, Y₃, or Y₄ can be N, and the remaining Y₁, Y₂, Y₃, or Y₄ can each be CR₃.

X can be —OCH₂— and Y₅ can be absent and each Y₁, Y₂, Y₃, and Y₄ can be CR₃.

X can be —OCH₂—; Y₅ can be absent and each Y₁, Y₂, Y₃, and Y₄ can be CR₃; R₁ can be C(O)OH; and R₂ can be C(O)OH.

X can be —OCH₂—, Y₅ can be absent, and where one of Y₁, Y₂, Y₃, or Y₄ can be N and the remaining Y₁, Y₂, Y₃, or Y₄ can each be CR₃.

X can be —OCH₂—; Y₅ can be absent, and where one of Y₁, Y₂, Y₃, or Y₄ can be N and the remaining Y₁, Y₂, Y₃, or Y₄ can each be CR₃; R₁ can be C(O)OH; and R₂ can be C(O)OH.

The composition of claim 30, wherein R₃ can be a halogen.

The composition of claim 30, wherein R₃ can be an optionally substituted aryl. The PTPase can be PTP1B.

The PTPase-mediated disorder or condition can be selected from type I diabetes, type II diabetes, obesity, cancer, autoimmune disease, allergic disorder, acute inflammation, chronic inflammation, metabolic syndrome, and osteoporosis.

In certain embodiments, R₁ is a 5- or 6-membered heterocycle. Preferred 5-membered heterocycles can include the following:

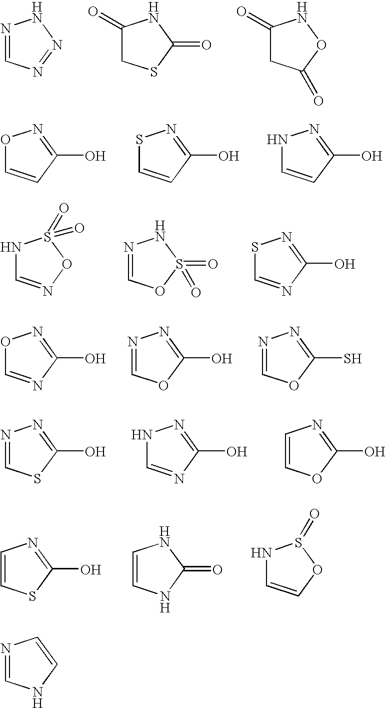

In certain embodiments, R₁ and R₂ are —C(O)OH or —C(O)OC₁₋₄alkyl.

In another aspect, X is —O—C₁₋₃alkylene-, —NR₈—C₁₋₃alkylene-, —S—C₁₋₃alkylene-, —SO C₁₋₃alkylene-, or —SO₂—C₁₋₃alkylene-, wherein any alkylene group is optionally substituted with one or more F, Cl, CN, OCF₃, OH, NH₂, NO₂, CHO, or Q. In certain embodiments, X is —O—CH₂—.

In another aspect, the fused heterocycle is benzothiophene or thienopyridine.

“Alkyl” refers to hydrocarbon chains that can contain 1 to 10 (preferably 1 to 6; more preferably 1 to 4) carbon atoms. Examples of alkyl include, but are not limited to, methyl, ethyl, propyl, butyl, pentyl, hexyl, heptyl, isopropyl, isobutyl, tert-butyl, isopentyl, neopentyl, octyl, or nonyl.

“Alkenyl” refers to a straight or branched hydrocarbon chain containing one or more (preferably 1-4; more preferably 1-2) double bonds and can contain 2 to 10 carbon atoms. Examples of alkenyl include vinyl, allyl, isopropenyl, pentenyl, hexenyl, heptenyl, 1-propenyl, 2-butenyl, or 2-methyl-2-butenyl.

“Alkynyl” refers to a straight or branched hydrocarbon chain containing one or more (preferably 1-4, or more preferably 1-2) triple bonds and can contain 2 to 10 carbon atoms. Examples of alkynyl include ethynyl, propargyl, 3-methyl-1-pentynyl, or 2-heptynyl.

“Cycloalkyl” refers to saturated or partly saturated monocyclic or polycyclic carbocyclic rings. Each ring can have from 3 to 10 carbon atoms. The term also can include a monocyclic or polycyclic ring fused to an aryl group or a heterocyclic group. Examples of cycloalkyl include cyclopropyl, cyclobutyl, cyclopentyl, cyclohexyl, cycloheptyl, cyclohexenyl, or cyclopentenyl.

“Heterocyclyl”, “heterocycle”, or “heterocyclic” refers to a saturated or partially saturated monocyclic or polycyclic ring system containing at least one heteroatom selected from N, O and S (including SO and SO₂). Each of the rings can have from 3 to 10 atoms, except where defined otherwise. Examples of this definition include tetrahydrofuran, piperazine, piperidine, tetrahydropyran, morpholine, pyrrolidine, or tetrahydrothiophene.

The term “aryl” means monocyclic-, polycyclic, biaryl or heterocyclic aromatic rings. Each ring can contain 5 to 6 atoms. The term also may describe one of the foregoing aromatic rings fused to a cycloalkyl or heterocyclic group. “Heterocyclic aromatic” and “heteroaryl” means a monocyclic or polycyclic aromatic rings containing at least one heteroatom selected from N, O and S (including SO and SO₂) in the perimeter of the ring. Each ring can contain 5 to 6 atoms. Examples of aryl include phenyl, naphthyl, biphenyl, indanyl, indenyl, tetrahydronaphthyl, dihydrobenzopyranyl, fluorenyl, pyrrolyl, pyrazolyl, imidazolyl, triazolyl, oxazolyl, isoxazolyl, oxadiazolyl, thiazoyl, thiadiazolyl, isothiazolyl, thienyl, thiophenyl, triazinyl, furanyl, pyridyl, tetrazolyl, pyrimidinyl, pyridazinyl, quinolyl, isoquinolyl, 2,3-dihydrobenzofuranyl, benzothiophenyl, 2,3-dihydrobenzothiophenyl, furo(2,3-b)pyridyl, isoquinolyl, dibenzofuran, benzisoxazolyl, benzoxazolyl, benzothiazolyl, benzimidazolyl, benzofuranyl, 4,5,6,7-tetrahydro-benzo[b]thiophenyl, indolyl, isoindolyl, 1,3-dihydro-isoindolyl, indazolyl, carbazolyl, 5H-dibenz[b,f]azepine, 10,11-dihydro-5H— dibenz[b,f]azepine, phenylpyridyl, phenylpyrimidinyl, phenylpyrazinyl, or phenypyridazinyl.

“Alkoxy” or alkyloxy” means an alkyl group as defined above having the indicated number of carbon atoms attached through an oxygen bridge. Examples include methoxy, ethoxy, or propyloxy. “Alkenyloxy” and “alkynyloxy” are similarly defined for alkenyl and alkynyl groups, respectively.

“Aryloxy” means an aryl group as defined above attached through an oxygen bridge. Examples include phenoxy or naphthyloxy.

“Cycloalkyloxy” and “heterocyclyloxy” are similiarly defined for cycloalkyl and heterocyclic groups, respectively.

Additional terms are similarly defined, following the convention that the last group in the term is the attachment point, unless is defined otherwise. For example, “arylalkenyl” represents an aryl group as defined above attached through an alkenyl group.

A salt of any of the compounds of formula (I) can be prepared. For example, a pharmaceutically acceptable salt can be formed when an amino-containing compound of this invention reacts with an inorganic or organic acid. Some examples of such an acid include hydrochloric acid, hydrobromic acid, hydroiodic acid, sulfuric acid, phosphoric acid, p-bromophenylsulfonic acid, carbonic acid, succinic acid, citric acid, benzoic acid, and acetic acid. Examples of pharmaceutically acceptable salts thus formed include sulfate, pyrosulfate bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, and maleate. A compound of this invention may also form a pharmaceutically acceptable salt when a compound of this invention having an acid moiety reacts with an inorganic or organic base. Such salts include those derived from inorganic or organic bases, e.g., alkali metal salts such as sodium, potassium, or lithium salts; alkaline earth metal salts such as calcium or magnesium salts; or ammonium salts or salts of organic bases such as morpholine, ethanol amine, choline, piperidine, pyridine, dimethylamine, or diethylamine salts. It should be recognized that a compound of the invention can contain chiral carbon atoms. In other words, it may have optical isomers or diastereoisomers.

An effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). An effective amount of a compound described herein can range from about 0.01-100 mg/kg, and more preferably from about 1-10 mg/kg. Effective doses will also vary, as recognized by those skilled in the art, dependent on route of administration, excipient usage, and the possibility of co-usage, pre-treatment, or post-treatment, with other therapeutic treatments.

The pharmaceutical composition may be administered via the parenteral route, including orally, topically, subcutaneously, intraperitoneally, intramuscularly, and intravenously. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well known to those familiar with the art, can be utilized as pharmaceutical excipients for delivery of the therapeutic compounds. Because some of the compounds described herein can have limited water solubility, a solubilizing agent can be included in the composition to improve the solubility of the compound. For example, the compounds can be solubilized in polyethoxylated castor oil (Cremophor EL®) and may further contain other solvents, e.g., ethanol.

A compound described herein can be formulated into dosage forms for other routes of administration utilizing conventional methods. For example, it can be formulated in a capsule, a gel seal, or a tablet for oral administration. Capsules may contain any standard pharmaceutically acceptable materials such as gelatin or cellulose. Tablets may be formulated in accordance with conventional procedures by compressing mixtures of a compound described herein with a solid carrier and a lubricant. Examples of solid carriers include starch and sugar bentonite. The compound can also be administered in a form of a hard shell tablet or a capsule containing a binder, e.g., lactose or mannitol, a conventional filler, and a tableting agent.

Inhibition of a PTPase may be determined by measuring turnover of various substrates, from small, phosphorylated organic compounds to endogenous phospho-peptides. McCain D F, Zhang Z Y: Assays for protein-tyrosine phosphatases. Methods Enzymol. (2002) 345: 507-518. Typical inhibition (Ki) values for the compounds disclosed herein ranged from 300 micromolar up to 10 micromolar. Some disorders or physiological conditions may be mediated by inhibition of a PTPase. A disorder or physiological condition that is mediated by PTPase refers to a disorder or condition wherein PTPase plays a role in either triggering the onset of the condition, or where inhibition of a particular PTPase affects signaling in such a way as to improve the condition. Examples of such disorders include, but are not limited to, type 1 and type 2 diabetes, obesity, cancer, autoimmune diseases, allergic disorders, acute and chronic inflammation, metabolic syndrome, and osteoporosis. Inhibitors of a specific PTPase can have therapeutic benefits in treating such disorders.

Protein tyrosine phosphatase 1B (PTP1B), a ˜50 kd intracelluar PTPase abundant in various human tissues, has been studied for its potential role as a negative regulator of insulin signaling. Some studies have shown that PTP1B is a negative regulator of insulin signaling. Mice deficient in PTP1B were healthy and showed increased insulin sensitivity and resistance to diet-induced obesity. These mice had lower glucose, insulin and triglyceride levels as well as improved insulin sensitivity as measured by glucose and insulin tolerance tests. Importantly, PTP1B has also been implicated in attenuation of leptin receptor signaling. PTP1B deficient mice were shown to be more sensitive to leptin, which may explain in part their resistance to weight gain when placed on a high fat diet. Thus, the main target tissues for PTP1B inhibition appear to be insulin action in muscle and liver, as well as leptin signaling in the brain, while the commercial diabetes drugs, the peroxisome proliferative activated receptor-gamma (PPAR-γ) agonist class of insulin sensitizers, target adipose tissue. Thus inhibition of PTP1B provides a unique target for regulating a variety of cellular responses important to human diseases related to obesity and type 2 diabetes.

The details of one or more embodiments are set forth in the accompanying drawings and the description below. Other features and advantages will be apparent from the description and drawings, and from the claims.

### DETAILED DESCRIPTION

Most of the compounds of the current invention may be prepared according to the following general synthetic scheme from commercially available starting materials, materials prepared as described in literature procedures, or new intermediates described in the schemes and experimental procedures. This general scheme covers many of the examples. More detailed synthetic methods are also set forth below.

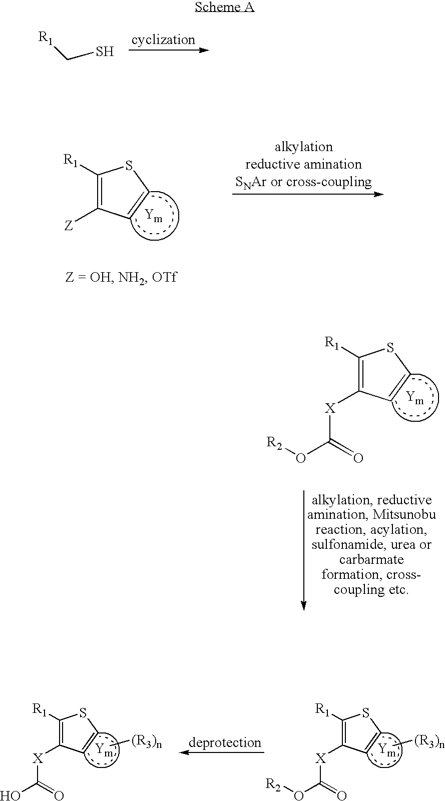

In Scheme A, a thiol such as a mercapto-acetic acid alkyl ester in the presence of a substituted heterocycle such as nicotinic acid cyclizes to afford a fused bicyclic thiophene. In the second step, an electronegative 3-substituent of the thiophene moiety can be alkylated or cross-coupled to form an alkoxy carboxylate or carboxylic acid at that position. In the third step, the heterocycle is substituted by various substituents according to general methods. In step four, the compound can be hydrolyzed to afford a terminal carboxylic acid at the 3-position.

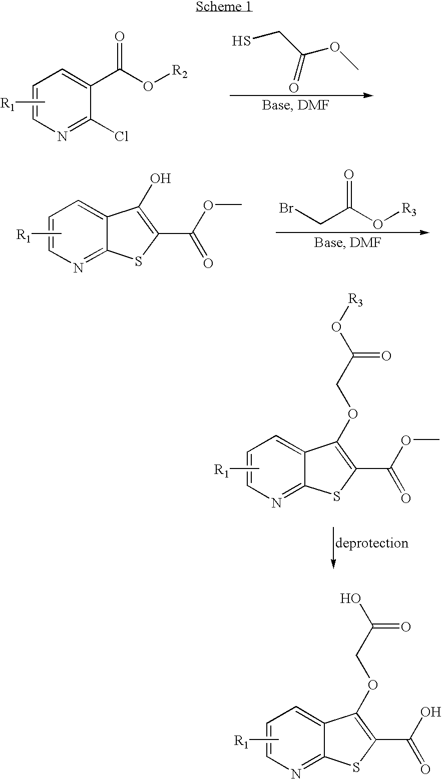

In Scheme 1, a mercapto-acetic acid alkyl ester in the presence of a substituted nicotinic acid ester cyclizes to afford a thienopyridine. In the second step, an electronegative 3-substituent of thiophene can be alkylated or cross-coupled to form an alkoxy carboxylate or carboxylic acid at that position. In the third step, the compound is hydrolyzed to afford terminal carboxylic acids at the 2- and 3-thiophene positions.

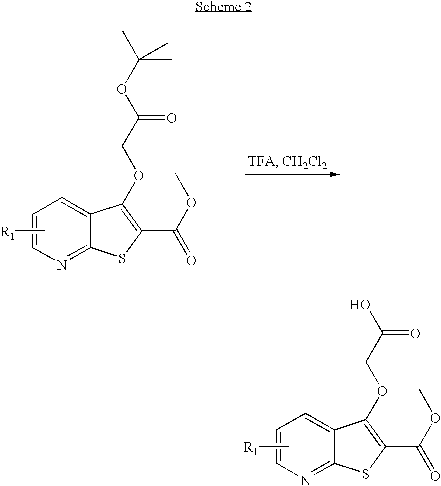

In Scheme 2, a 3-thienopyridine substituent is selectively hydrolyzed to afford a terminal carboxylic acid at that position.

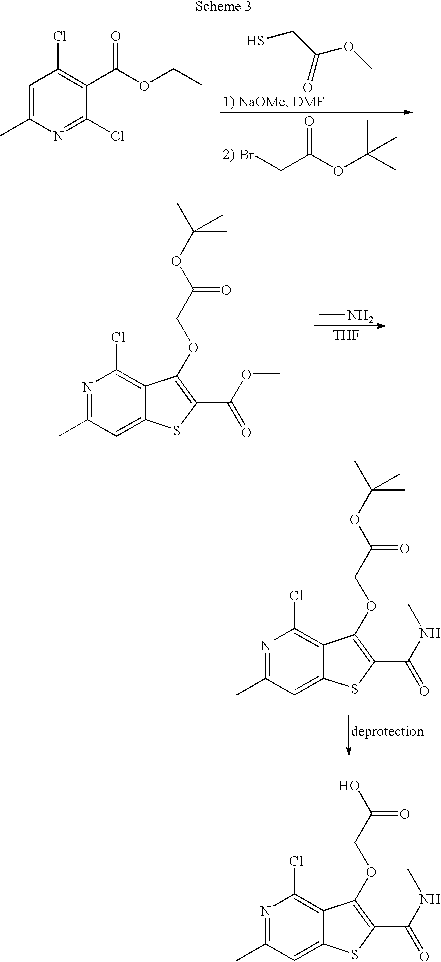

In Scheme 3, a mercapto-acetic acid alkyl ester in the presence of a substituted nicotinic acid ester cyclizes to afford a thienopyridine. In the second part of step one, the 3-position of thiophene is alkylated or cross-coupled to form an alkoxy carboxylate or carboxylic acid. In step two, a carboxylate at the 2-position reacts with an amine to form an amide. In the third step, the 3-position is hydrolyzed to afford a terminal carboxylic acid.

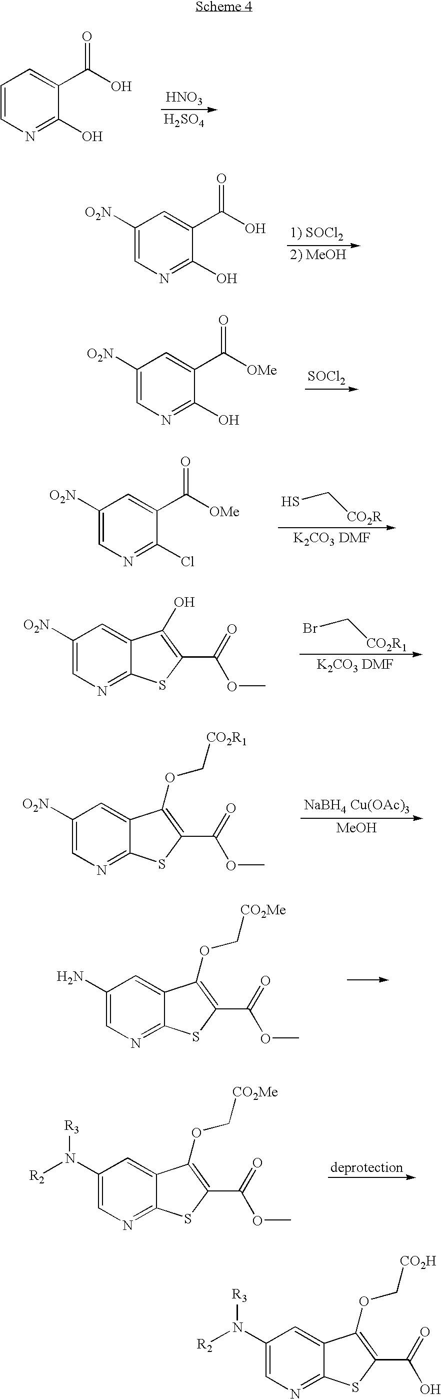

In Scheme 4, 2-hydroxy nicotinic acid is substituted with a nitro group by conventional methods. In step two, the carboxylic acid moiety is alkylated to form an ester. In step three, the two-hydroxy moiety is halogenated. In step four, the nicotinic acid reacts with mercapto-acetic acid alkyl ester to form a thienopyridine. In step five, the three-thiophene position is alkylated to form an alkoxycarboxylate. The nitro group of the pyridine is reduced in step six. In step seven, the amine is substituted by conventional methods. The compound can be hydrolyzed to afford terminal carboxylic acids at the 2- and 3-positions in step eight.

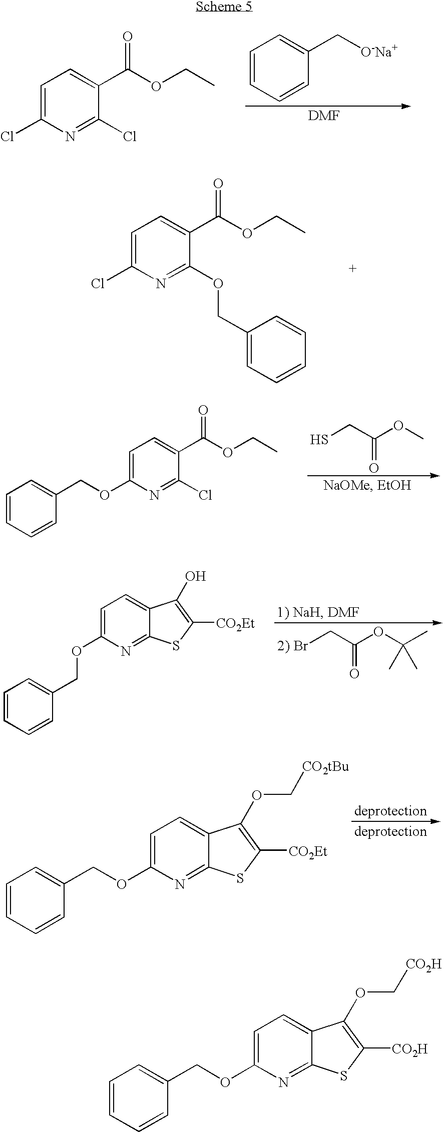

In Scheme 5, a nicotinic acid ester is substituted with an arylalkoxy group to yield two products (2- and 6-substitution). The 6-substituted product reacts with a mercapto-acetic acid ester to cyclize into a thienopyridine in step two. In the third step, the 3-thiophene position is substituted with an alkyloxy carboxylate, which is hydrolyzed along with an ester at the 2-position to terminal carboxylic acids in step four.

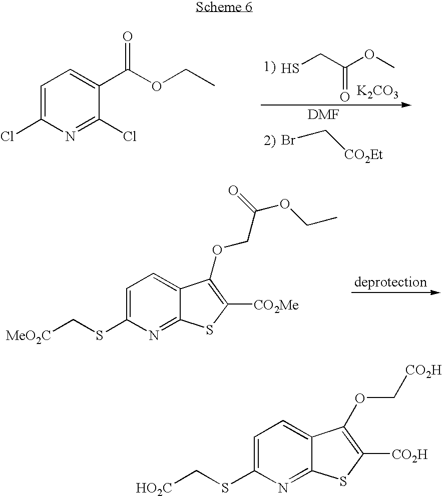

In Scheme 6, a nicotinic acid ester reacts with mercapto acetic acid alkyl ester to form a thienopyridine. In addition, a 6-pyridine chloro substituents reacts with the mercapto moiety to yield an alkylthiocarboxylate. In the second part of step one, 3-hydroxy thiophene is alkylated to form an alkoxy carboxylate. In step two, the compound is hydrolyzed to afford terminal carboxylic acids at the 2- and 3-positions, as well as at the 6-pyridine position.

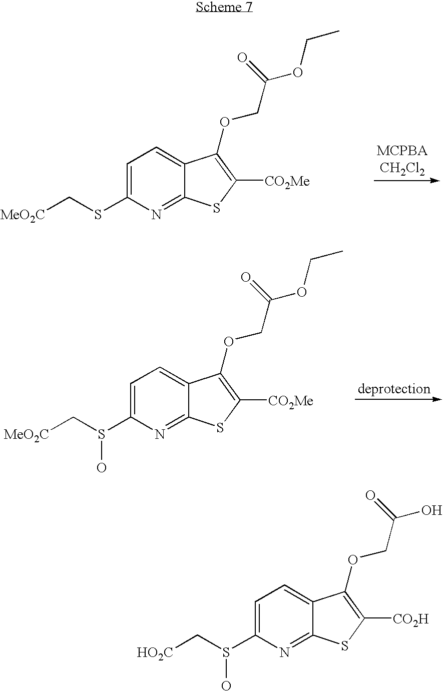

In Scheme 7, a 6-pyridine (of a thienopyridine) sulfanyl alkyl carboxylate is oxidized to a sulfinyl. Protective groups on the 2- and 3-substituents of the thiophene, as well as the sulfinyl alkyl carboxylate, can be hydrolyzed to afford terminal carboxylic acids.

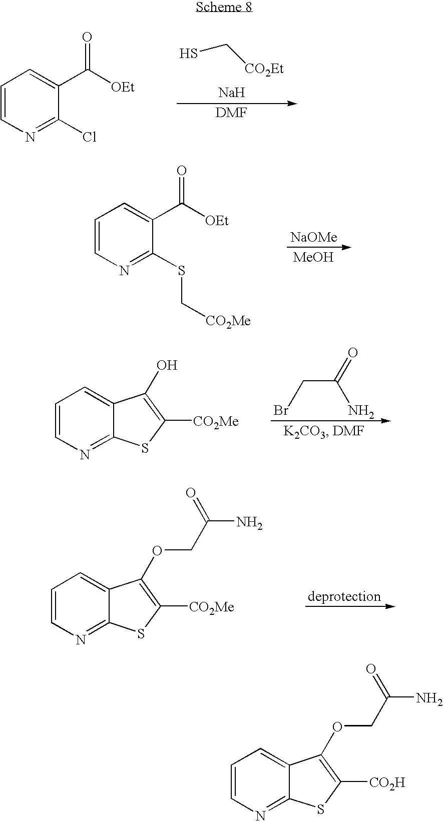

In Scheme 8, a mercapto-acetic acid alkyl ester in the presence of a substituted nicotinic acid ester forms a sulfanyl alkyl carboxylate at the 2-pyridine position. In step two, the compound cyclizes to form a thienopyridine. In step three, the 3-thiophene position is alkylated to form a carbamoyl alkoxy group. The compound can be hydrolyzed to afford a carboxylic acid at the 2-position in step four.

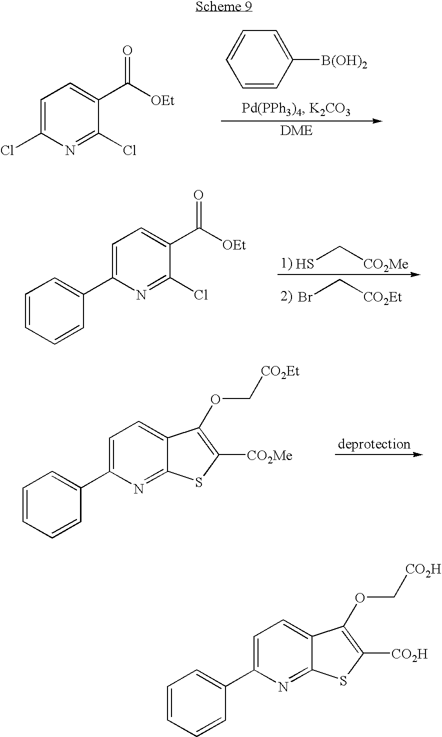

In Scheme 9, a 2,6-dichloronicotinic acid ester is substituted with phenyl at the 6-position following conventional methods. In step two, the substituents at the 2- and 3-positions react with a mercapto acetic acid alkyl ester to cyclize into a thienopyridine, which can be alkylated to an alkoxy carboxylate at the 3-thiophene position. In step three, the compound is hydrolyzed to afford terminal carboxylic acids at the 2- and 3-thiophene positions.

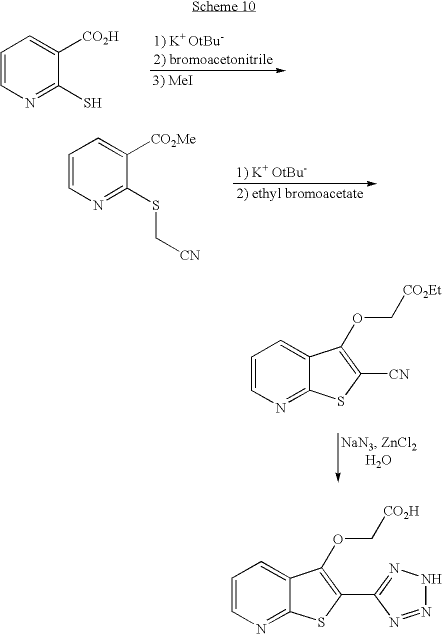

In Scheme 10, 2-mercaptonicotinic acid reacts with bromoacetonitrile and alkyl iodine to afford 2-cyanomethylsulfanyl-nicotinic acid alkyl ester. In step two, the resulting compound cyclizes to a thienopyridine and reacts with ethyl bromoacetate to form an alkoxy carboxylate at the thiophene 3-position. In step three, the cyano group reacts with sodium azide to yield a tetrazole at the 2-thiophene position. The 3-thiophene substituent can be hydrolyzed to a terminal carboxylic acid.

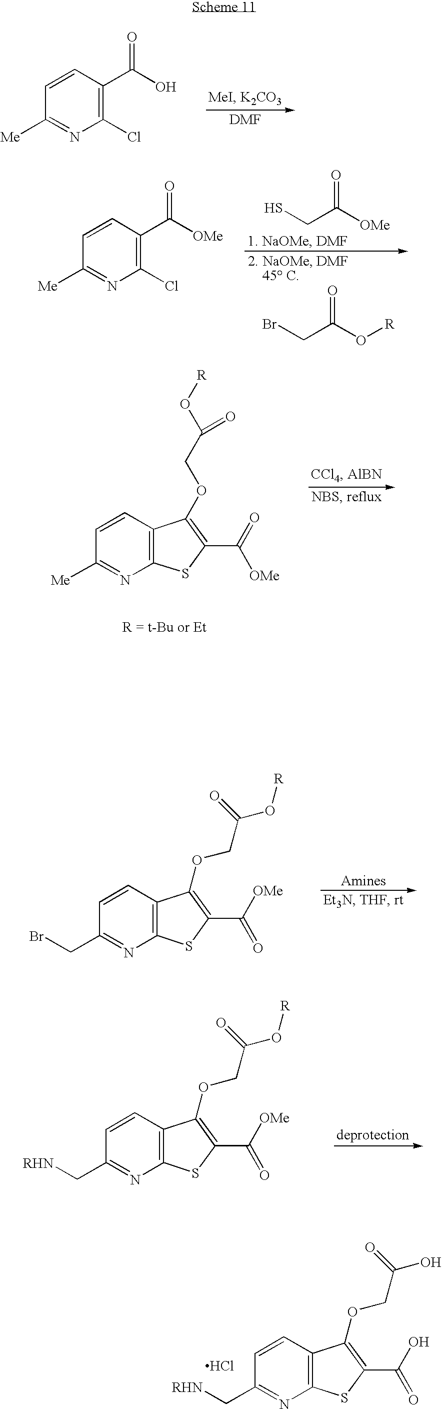

In Scheme 11, step one, nicotinic acid alkyl ester is formed from nicotinic acid. In step two, the resulting compound reacts with mercapto acetic acid ester to cyclize into a thienopyridine, which is then alkylated into an alkoxycarboxylate at the 3-thiophene position. In step three, a methyl group at the 6-pyridine position is halogenated. In step four, the halogen reacts with an amine to form a substituted amine. The resulting compound is hydrolyzed to yield terminal carboxylic acids at the 2- and 3-thiophene positions.

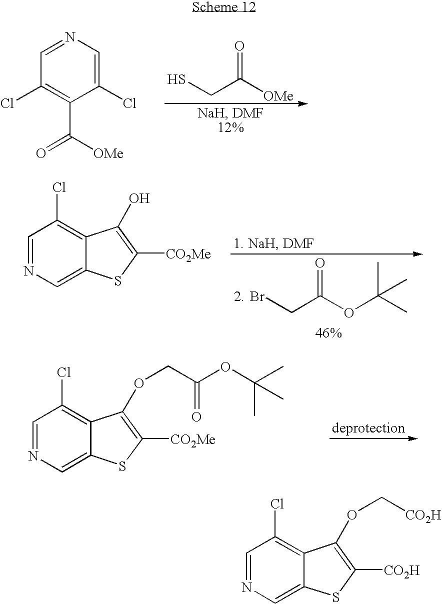

In Scheme 12, a nicotinic acid ester reacts with mercapto acetic acid alkyl ester to form a thienopyridine. In step two, a 3-hydroxy thiophene substituent is alkylated to form an alkoxy carboxylate. In step three, the compound is hydrolyzed to afford terminal carboxylic acids at the 2- and 3-thiophene positions.

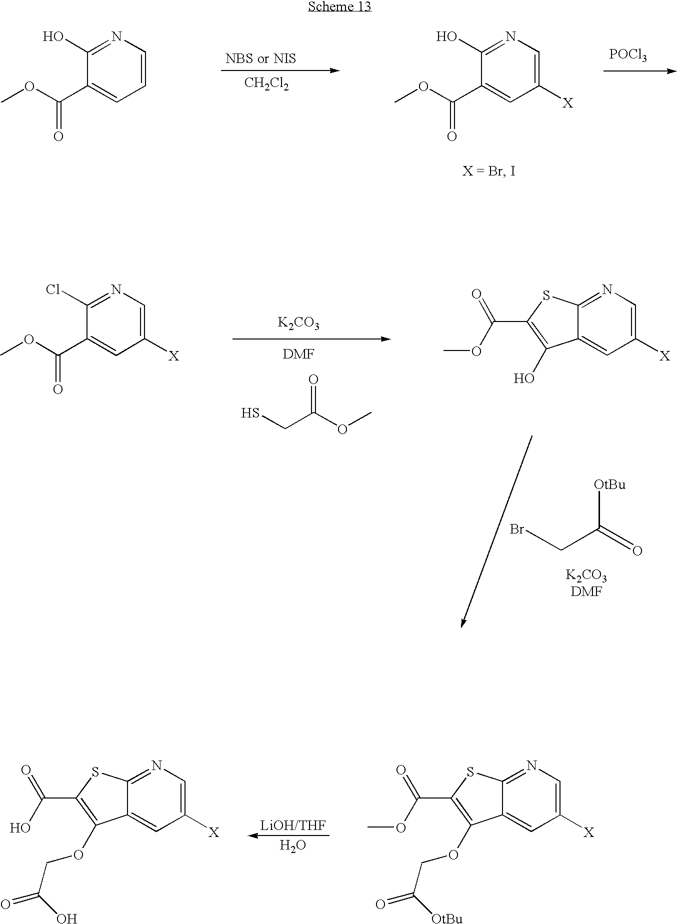

In Scheme 13, step one, a nicotinic acid ester reacts with a halogenating reagent to afford a halogen substituent meta- to the acid ester. A hydroxy substituent is substituted with a chlorine in step two. In step three, the ester reacts with mercapto acetic acid alkyl ester to form a thienopyridine. A 3-hydroxy thiophene substituent is alkylated to form an alkoxy carboxylate in step four. In step five, the compound is hydrolyzed to afford terminal carboxylic acids at the 2- and 3-thiophene positions.

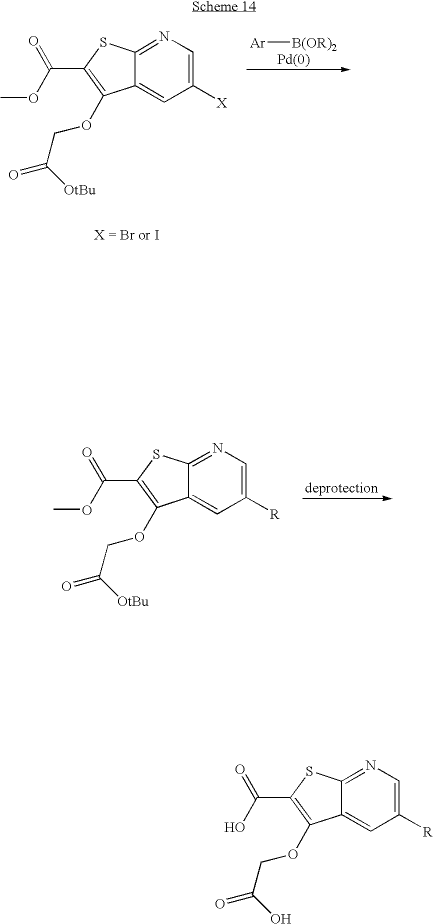

In Scheme 14, a halogen substituent on the pyridine of a thienopyridine is substituted with a group such as aryl, alkene, or alkyl following conventional methods. The compound is hydrolyzed in step two to afford terminal carboxylic acids at the 2- and 3-thiophene positions.

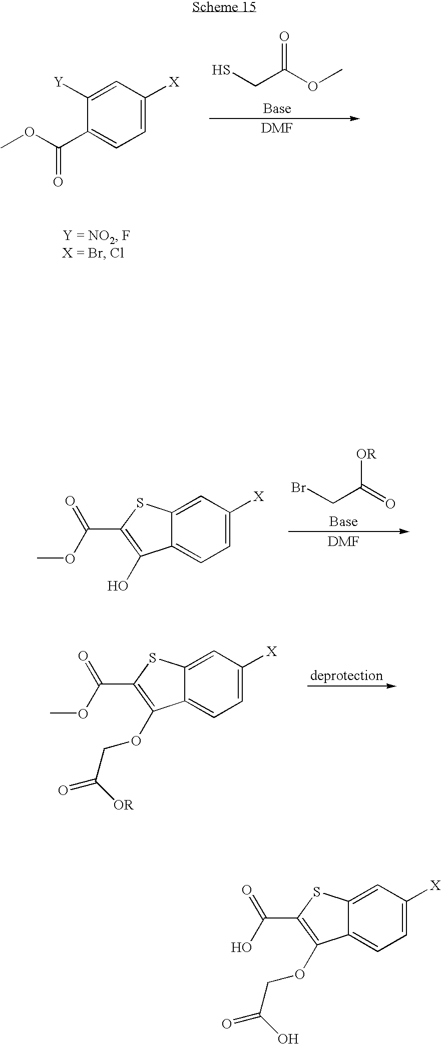

In Scheme 15, a substituted benzoic acid alkyl ester cyclizes in the presence of a mercapto acetic acid alkyl ester to a benzothiophene. A 3-hydroxy thiophene substituent is alkylated to form an alkoxy carboxylate in step two. In step three, the compound is hydrolyzed to afford terminal carboxylic acids at the 2- and 3-thiophene positions.

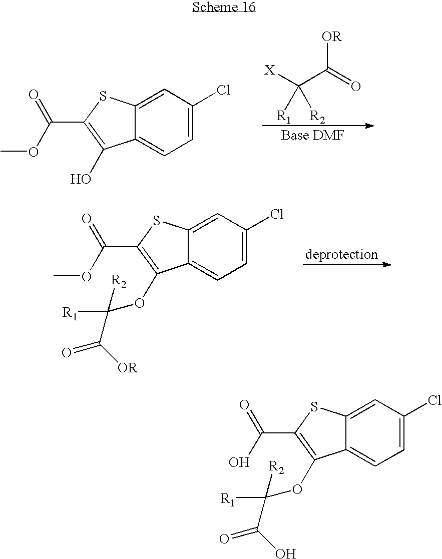

In Scheme 16, a 3-hydroxybenzothiophene substituent is alkylated to form a substituted alkoxy carboxylate. In step two, the compound is hydrolyzed to afford terminal carboxylic acids at the 2- and 3-thiophene positions.

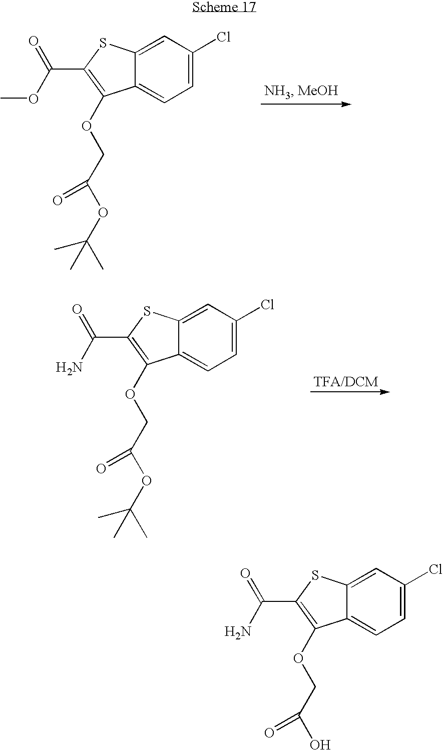

In Scheme 17, a 2-alkylester thiophene substituent of benzothiophene reacts with ammonia to form an amide. The resulting amide can be alkylated following conventional methods, and a 3-alkoxy carboxylate can be hydrolyzed to a carboxyalkoxy substituent.

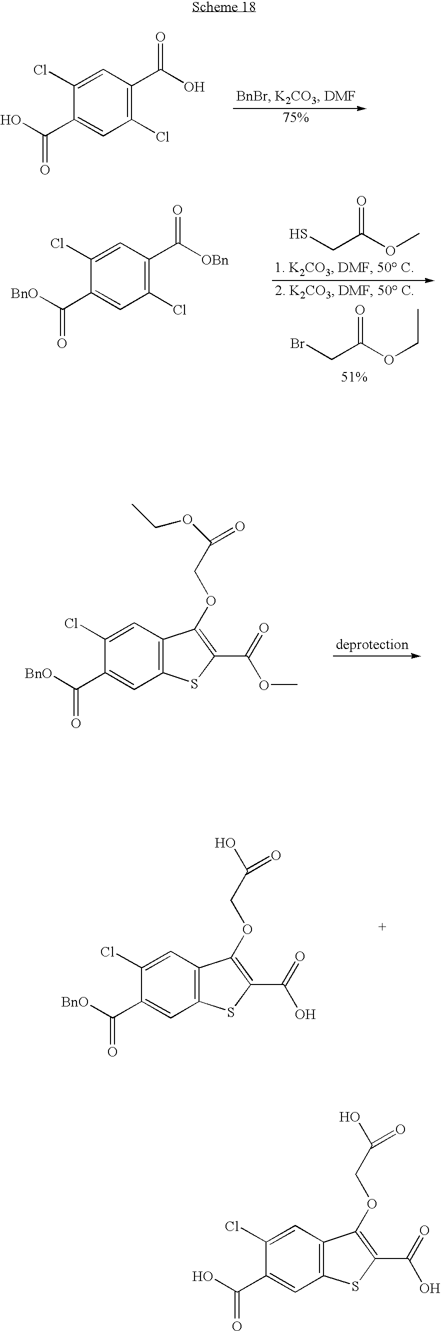

In Scheme 18, a substituted terephthalic acid reacts with benzylbromide to form benzoic acid ester substituents. The resulting compound reacts with a mercapto acetic acid alkyl ester to form benzothiophene, which can be alkylated to form an alkoxy carboxylate substituent at the thiophene 3-position. In step three, hydrolysis affords two compounds: one with a deprotected carboxylic acid on the benzene ring as well as at the 2- and 3-thiophene positions, and one that has been deprotected only at the 2- and 3-thiophene positions.

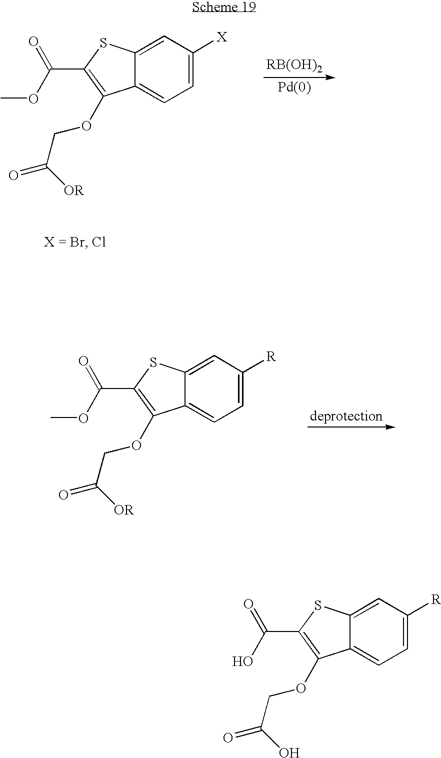

In Scheme 19, a halogen substituent on the benzene of a benzothiophene is substituted with a group such as aryl or alkyl following conventional methods. The resulting compound can be hydrolyzed to afford terminal carboxylic acids on the 2- and 3-thiophene positions.

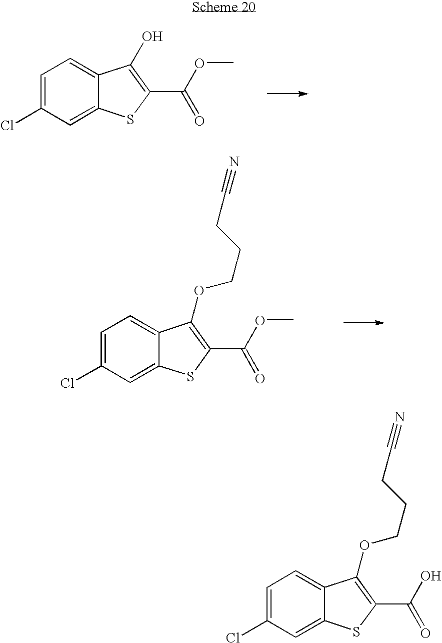

In Scheme 20, a 3-hydroxy thiophene substituent of benzothiophene is alkylated to form a 3-cyanoalkoxy group. The resulting compound can be hydrolyzed to afford a terminal carboxylic acid on the 2-thiophene position.

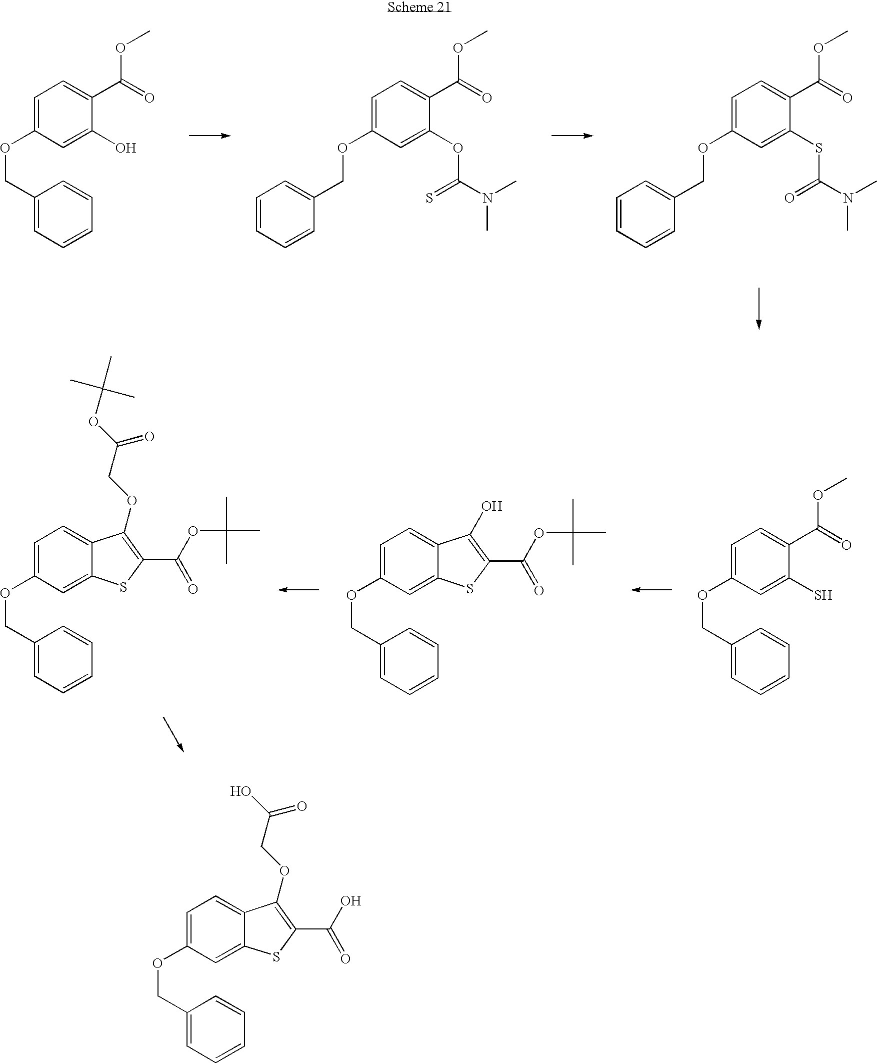

In Scheme 21, the 2-hydroxy group of a benzoic acid alkyl ester reacts with a thiocarbamoyl halide to afford a thiocarbamoyloxy substituent at that position. In step two, the compound rearranges to afford a 2-carbamoylsulfanyl group at the 2-position. In the third step, the compound is hydrolyzed to a 2-mercapto group. The resulting compound cyclizes in the presence of sodium methoxide to afford a 2-carboxylic acid ester, 3-hydroxy benzothiophene. In step five, the 3-hydroxy group is alkylated to yield an alkoxy carboxylate group. The resulting compound is hydrolyzed to afford terminal carboxylic acids at the 2- and 3-thiophene positions.

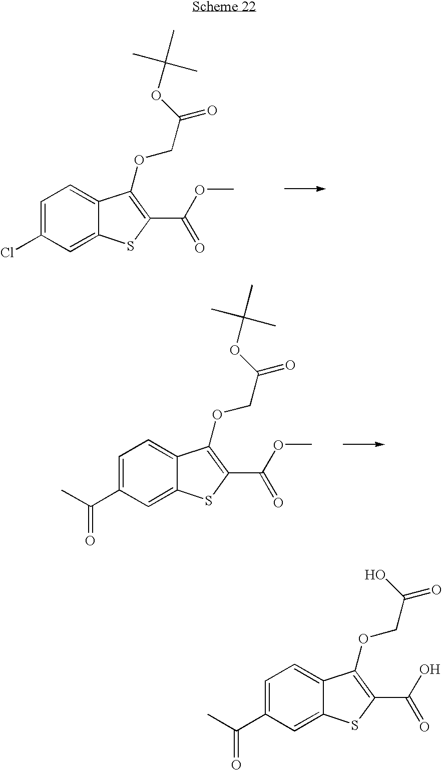

In Scheme 22, a halogen substituent on the benzene group of benzothiophene is converted to an acyl group by conventional methods. The resulting compound is hydrolyzed to afford terminal carboxylic acids at the 2- and 3-thiophene positions.

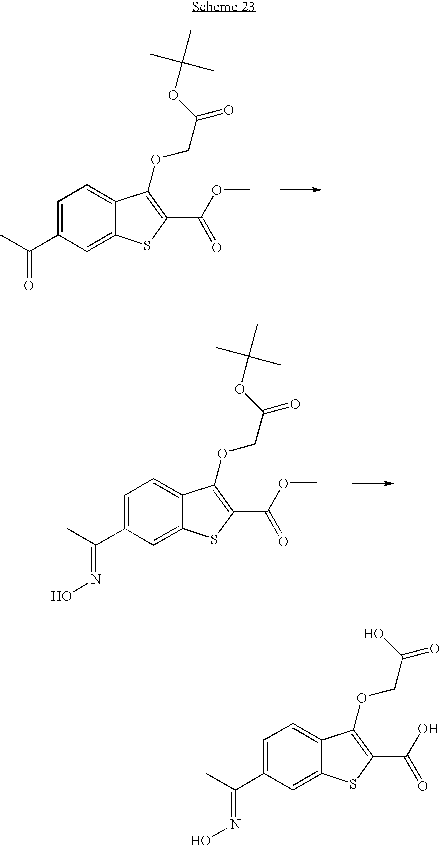

In Scheme 23, an acetyl group on the benzene of a benzothiophene is converted to a hydroxyimine following reaction with hydroxylamine hydrochloride. In step two, the resulting compound is hydrolyzed to afford terminal carboxylic acids at the 2- and 3-thiophene positions.

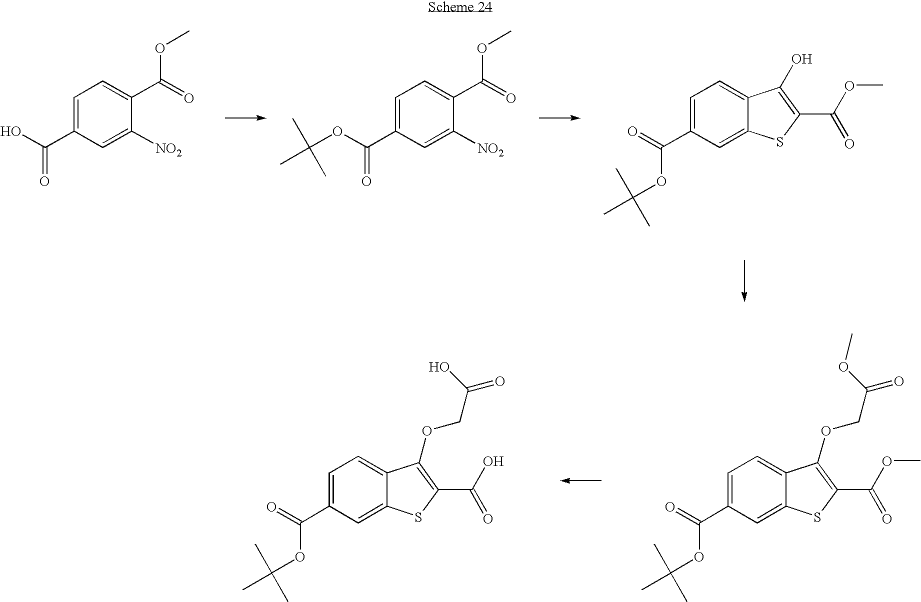

In Scheme 24, nitroterephthalic acid methyl ester reacts with an alkene such as isobutylene to form another ester on the benzene moiety. In step two, the compound cyclizes in the presence of a mercapto acetic acid alkyl ester to form benzothiophene. The 3-hydroxy substituent is alkylated to form an alkoxy carboxylate in step three. In step four, the compound is hydrolyzed to afford terminal carboxylic acids on the 2- and 3-thiophene positions.

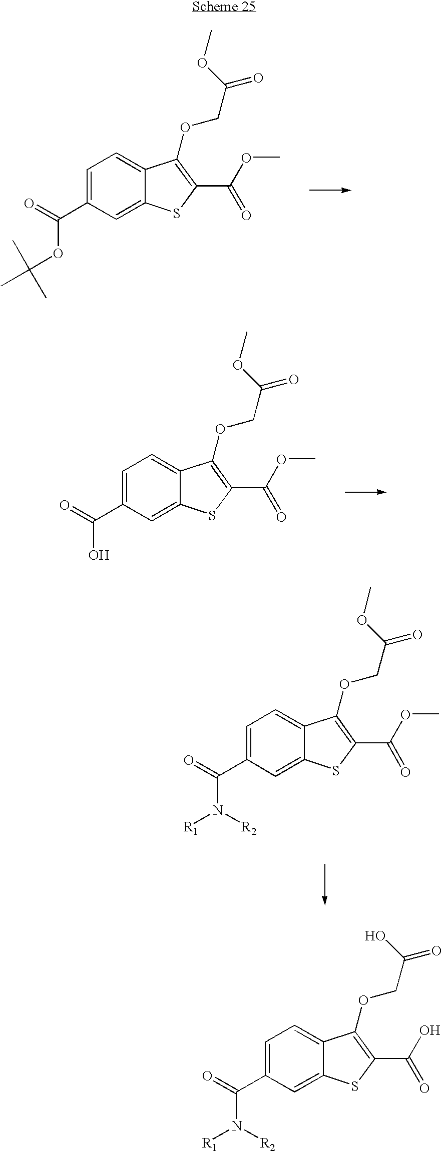

In Scheme 25, a tert-butyl ester substituent on the benzene moiety of benzothiophene is hydrolyzed to afford a carboxylic acid. The carboxylic acid is converted to an amide following conventional procedures in step two. In step three, the compound is hydrolyzed to afford terminal carboxylic acids at the 2- and 3-thiophene positions.

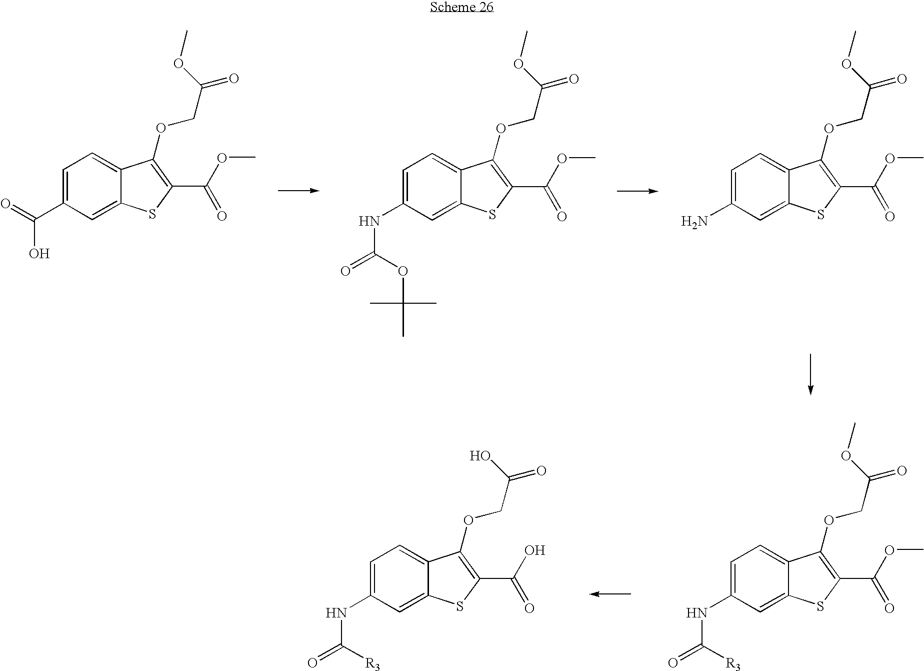

In Scheme 26, a carboxylic acid substituent on the benzene moiety of benzothiophene is converted to an alkoxycarbonylamino group. In step two, the alkoxycarbonylamino group is hydrolyzed to an amine. In step three, the amine is acylated to form an amide. The compound can be hydrolyzed to afford terminal carboxylic acids at the 2- and 3-thiophene positions.

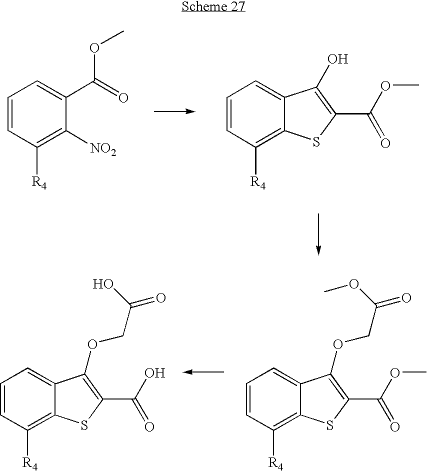

In Scheme 27, nitrobenzoic acid alkyl ester cyclizes in the presence of mercapto acetic acid alkyl ester to form 3-hydroxy, 2-alkyl ester benzothiophene. The 3-hydroxy group can be alkylated to form an alkoxy carboxylate in step two. In step three, the compound is hydrolyzed to form terminal carboxylic acids at the 2- and 3-positions.

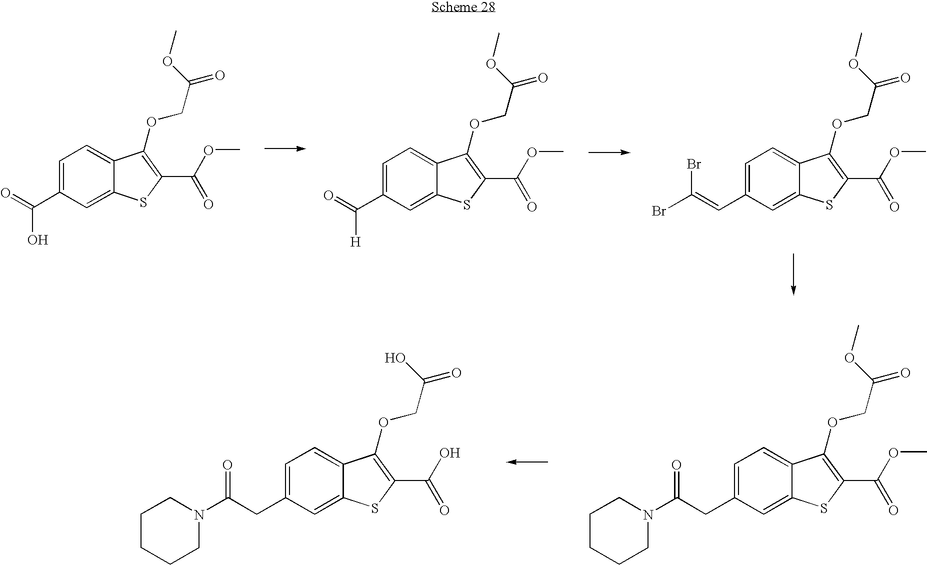

In Scheme 28, a carboxylic acid substituent on the benzene moiety of benzothiophene is converted to a formyl substituent following conventional methods. In step two, the formyl group is converted to a dihalovinyl group. The compound reacts with piperidine in an alkylformamide to form an oxo alkyl piperidine substituent. In step four, hydrolysis affords terminal carboxylic acids at the 2- and 3-thiophene positions.

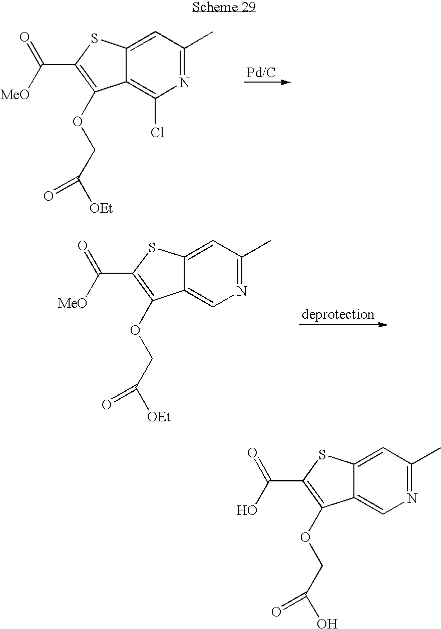

In Scheme 29, a halogen substituent on the pyridine moiety of thienopyridine is removed following treatment with hydrogen and palladium/carbon. In step two, hydrolysis affords terminal carboxylic acids at the 2- and 3-thiophene positions.

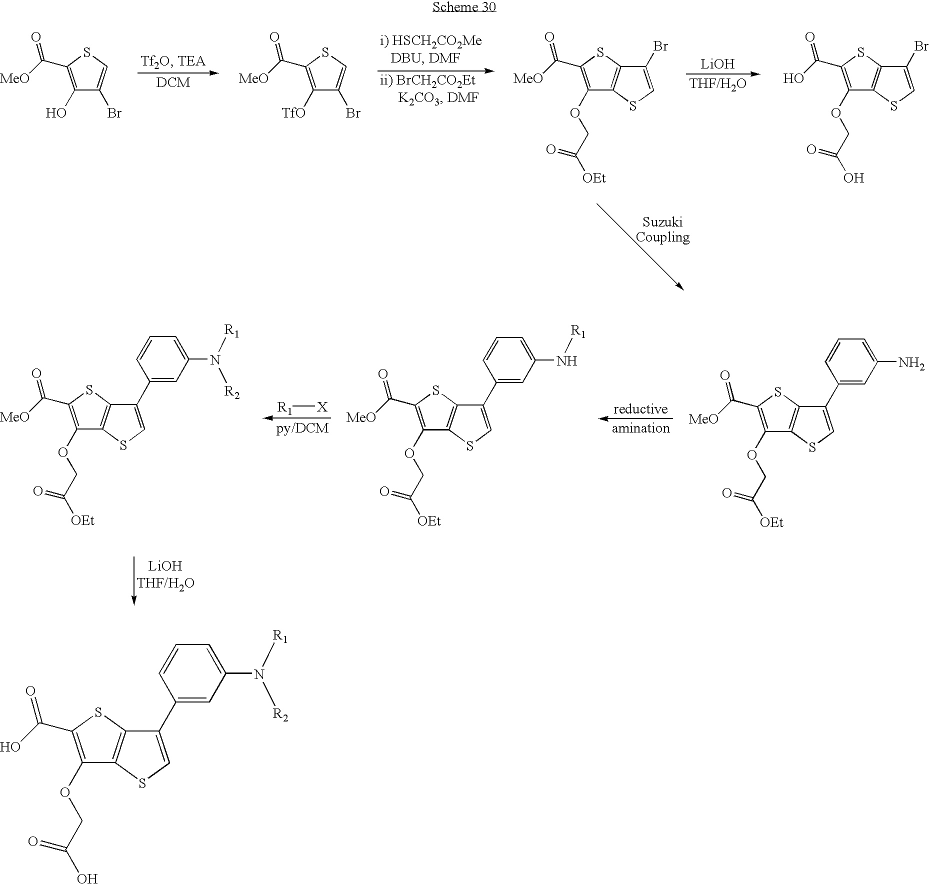

In Scheme 30, a 3-hydroxy thiophene substituent is substituted with triflate. In step two, the thiophene reacts with a mercapto-acetic acid alkyl ester and cyclizes into a bicyclic thienothiophene. The triflate can also be substituted with an alkoxycarboxylate. In step three, a 6-halo substituent is cross-coupled to form an aminophenyl at that position. The amine can be substituted by reductive amination in step four, and further substituted by reaction with a halogenated compound in step five. Hydrolysis yields terminal carboxylic acids at the 2- and 3-positions.

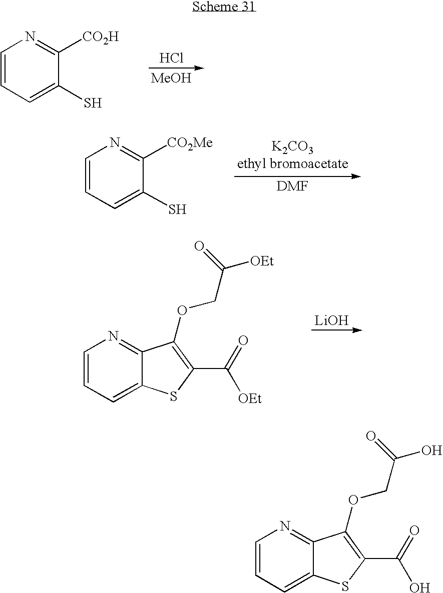

In Scheme 31, a 2-carboxy substituent on a pyridine is esterified. In step 2, the esterified product is condensed with ethyl bromoacetate. In step 3, the diester is saponified.

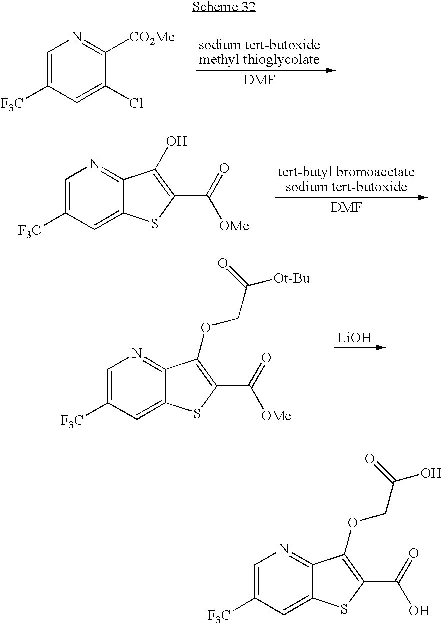

In Scheme 32, a 2-carbomethoxy substituted pyridine is condensed with methyl thioglycolate. In step 2, the hydroxy group of the resultant bicyclic product is alkylated with tert-butyl bromoacetate. In step 3, the diester is saponified.

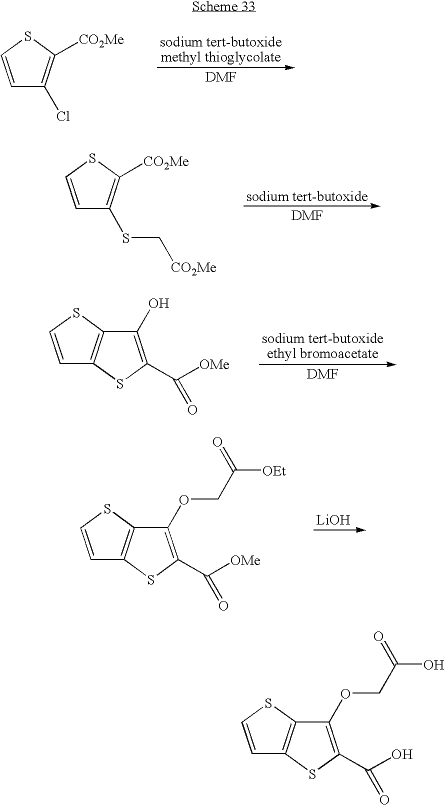

In Scheme 33, a 3-chloro substituent on a thiophene is replaced with methyl thioglycolate. In step 2, the diester is cyclized to form a thienothiophene. In step 3, the hydroxy group of the thienothiophene is alkylated with tert-butyl bromoacetate. In step 4, the diester is saponified.

Some compounds of formula (I) are set forth below:

3-Carboxymethoxy-6-methyl-thieno[2,3-b]pyridine-2-carboxylic acid, 3-Carboxymethoxy-thieno[2,3-b]pyridine-2-carboxylic acid methyl ester, 3-tert-Butoxycarbonylmethoxy-4-chloro-6-methyl-thieno[3,2-c]pyridine-2-carboxylic acid methyl ester, 4-Chloro-6-methyl-2-methylcarbamoyl-thieno[3,2-c]pyridin-3-yloxy)-acetic acid tert-butyl ester, (4-Chloro-6-methyl-2-methylcarbamoyl-thieno[3,2-c]pyridin-3-yloxy)-acetic acid, 5-Acetylamino-3-carboxymethoxy-thieno[2,3-b]pyridine-2-carboxylic acid, 3-Carboxymethoxy-5-nitro-thieno[2,3-b]pyridine-2-carboxylic acid, 5-Benzylamino-3-carboxymethoxy-thieno[2,3-b]pyridine-2-carboxylic acid, 3-Carboxymethoxy-5-diethylamino-thieno[2,3-b]pyridine-2-carboxylic acid, 6-Benzyloxy-3-carboxymethoxy-thieno[2,3-b]pyridine-2-carboxylic acid, 3-Carboxymethoxy-6-carboxymethylsulfanyl-thieno[2,3-b]pyridine-2-carboxylic acid, 6-Carboxymethanesulfinyl-3-carboxymethoxy-thieno[2,3-b]pyridine-2-carboxylic acid, 3-Carbamoylmethoxy-thieno[2,3-b]pyridine-2-carboxylic acid, 3-Carboxymethoxy-6-phenyl-thieno[2,3-b]pyridine-2-carboxylic acid,

(2-Cyano-thieno[2,3-b]pyridin-3-yloxy)-acetic acid, [2-(2H-Tetrazol-5-yl)-thieno[2,3-b]pyridin-3-yloxy]-acetic acid, 3-Carboxymethoxy-6-(isobutylamino-methyl)-thieno[2,3-b]pyridine-2-carboxylic acid hydrochloride, 6-(Benzylamino-methyl)-3-carboxymethoxy-thieno[2,3-b]pyridine-2-carboxylic acid hydrochloride, 3-Carboxymethoxy-6-[(2-methoxy-ethylamino)-methyl]-thieno[2,3-b]pyridine-2-carboxylic acid hydrochloride, 3-Carboxymethoxy-6-[(2-thiophen-3-yl-ethylamino)-methyl]-thieno[2,3-b]pyridine-2-carboxylic acid hydrochloride, 6-(Benzoylamino-methyl)-3-carboxymethoxy-thieno[2,3-b]pyridine-2-carboxylic acid hydrochloride, 6-(Benzenesulfonylamino-methyl)-3-carboxymethoxy-thieno[2,3-b]pyridine-2-carboxylic acid, 3-Carboxymethoxy-4-chloro-thieno[2,3-c]pyridine-2-carboxylic acid, 5-Bromo-3-carboxymethoxy-thieno[2,3-b]pyridine-2-carboxylic acid, 3-Carboxymethoxy-5-iodo-thieno[2,3-b]pyridine-2-carboxylic acid, 3-Carboxymethoxy-5-styryl-thieno[2,3-b]pyridine-2-carboxylic acid, 3-Carboxymethoxy-5-phenyl-thieno[2,3-b]pyridine-2-carboxylic acid, 5-Benzyl-3-carboxymethoxy-thieno[2,3-b]pyridine-2-carboxylic acid, 3-Carboxymethoxy-6-chloro-benzo[b]thiophene-2-carboxylic acid, 6-Bromo-3-carboxymethoxy-benzo[b]thiophene-2-carboxylic acid, 6-Chloro-3-(1-methoxycarbonyl-ethoxy)-benzo[b]thiophene-2-carboxylic acid methyl ester, 3-(1-Carboxy-ethoxy)-6-chloro-benzo[b]thiophene-2-carboxylic acid, 6-Chloro-3-(ethoxycarbonyl-fluoro-methoxy)-benzo[b]thiophene-2-carboxylic acid methyl ester, 3-(Carboxy-fluoro-methoxy)-6-chloro-benzo[b]thiophene-2-carboxylic acid, (2-Carbamoyl-6-chloro-benzo[b]thiophen-3-yloxy)-acetic acid tert-butyl ester, 3-Carbamoylmethoxy-6-chloro-benzo[b]thiophene-2-carboxylic acid amide, (2-Carbamoyl-6-chloro-benzo[b]thiophen-3-yloxy)-acetic acid, 3-Carboxymethoxy-5-chloro-benzo[b]thiophene-2,6-dicarboxylic acid 6-benzyl ester, 3-Carboxymethoxy-5-chloro-benzo[b]thiophene-2,6-dicarboxylic acid, 3-Carboxymethoxy-6-phenylbenzo[b]thiophene-2-carboxylic acid, 6-Benzyl-3-carboxymethoxy-benzo[b]thiophene-2-carboxylic acid, 3-Carboxymethoxy-6-thiophen-3-yl-benzo[b]thiophene-2-carboxylic acid, 3-Carboxymethoxy-6-thiophen-2-yl-benzo[b]thiophene-2-carboxylic acid, 3-Carboxymethoxy-6-(4-hydroxyphenyl)-benzo[b]thiophene-2-carboxylic acid, 3-Carboxymethoxy-6-(3-hydroxy-phenyl)-benzo[b]thiophene-2-carboxylic acid, 3-Carboxymethoxy-6-(4-nitro-phenyl)-benzo[b]thiophene-2-carboxylic acid, 6-(4-Aminophenyl)-3-carboxymethoxy-benzo[b]thiophene-2-carboxylic acid, 6-(3-Amino-phenyl)-3-carboxymethoxy-benzo[b]thiophene-2-carboxylic acid, 3-Carboxymethoxy-6-(4-dimethylaminophenyl)-benzo[b]thiophene-2-carboxylic acid, 3-Carboxymethoxy-6-(4-cyanophenyl)-benzo[b]thiophene-2-carboxylic acid, 3-Carboxymethoxy-6-(2-methoxyphenyl)-benzo[b]thiophene-2-carboxylic acid, 3-Carboxymethoxy-6-phenylamino-benzo[b]thiophene-2-carboxylic acid, 3-Carboxymethoxy-6-naphthalen-1-yl-benzo[b]thiophene-2-carboxylic acid, 3-Carboxymethoxy-6-(4-methoxy-phenyl)-benzo[b]thiophene-2-carboxylic acid dilithio salt, 3-Carboxymethoxy-6-(3-formyl-thiophen-2-yl)-benzo[b]thiophene-2-carboxylic acid, 3-Carboxymethoxy-6-(3-hydroxymethyl-thiophen-2-yl)-benzo[b]thiophene-2-carboxylic acid, 6-Chloro-3-(3-cyanopropoxy)benzo[b]thiophene-2-carboxylic acid, 6-Benzyloxy-3-carboxymethoxy-benzo[b]thiophene-2-carboxylic acid, 6-Acetyl-3-carboxymethoxy-benzo[b]thiophene-2-carboxylic acid, 3-Carboxymethoxy-6-(1-hydroxyiminoethyl)benzo[b]thiophene-2-carboxylic acid, 3-Carboxymethoxy-6-(3-fluorophenyl)benzo-[b]thiophene-2-carboxylic acid, 3-Carboxymethoxy-6-(2-fluorophenyl)-benzo[b]thiophene-2-carboxylic acid, 3-Carboxymethoxy-benzo[b]thiophene-2,6-dicarboxylic acid 6-tert-butyl ester, 6-Carbamoyl-3-carboxymethoxy-benzo[b]thiophene-2-carboxylic acid, 3-Carboxymethoxy-6-dimethylcarbamoyl-benzo[b]thiophene-2-carboxylic acid, 6-Benzylcarbamoyl-3-carboxymethoxy-benzo[b]thiophene-2-carboxylic acid, 6-(Benzylmethylcarbamoyl)-3-carboxymethoxy-benzo[b]thiophene-2-carboxylic acid, 3-Carboxymethoxy-6-(2-methyl-5-phenyl-2H-pyrazol-3-ylcarbamoyl)-benzo[b]thiophene-2-carboxylic acid, 3-Carboxymethoxy-6-(3-methyl-isothiazol-5-ylcarbamoyl)-benzo[b]thiophene-2-carboxylic acid, 3-Carboxymethoxy-6-(thiazol-2-ylcarbamoyl)benzo[b]thiophene-2-carboxylic acid, 3-carboxymethoxy-6-(5-methyl-1-phenyl-1H-pyrazol-3-ylcarbamoyl)-benzo[b]thiophene-2-carboxylic acid, 3-Carboxymethoxy-6-(3-methylisoxazol-5-ylcarbamoyl)-benzo[b]thiophene-2-carboxylic acid, 6-tert-Butoxycarbonylamino-3-carboxymethoxy-benzo[b]thiophene-2-carboxylic acid, 6-Benzoylamino-3-carboxymethoxybenzo[b]thiophene-2-carboxylic acid, 6-(3-Benzylureido)-3-carboxymethoxybenzo[b]thiophene-2-carboxylic acid, 3-Carboxymethoxy-7-chloro-benzo[b]thiophene-2-carboxylic acid, 3-Carboxymethoxy-7-methyl-benzo[b]thiophene-2-carboxylic acid, 3-Carboxymethoxy-6-(2-oxo-2-piperidin-1-yl-ethyl)-benzo[b]thiophene-2-carboxylic acid, 3-Ethoxycarbonylmethoxy-6-methyl-thieno[3,2-c]pyridine-2-carboxylic acid methyl ester, 3-Carboxymethoxy-6-methyl-thieno[3,2-c]pyridine-2-carboxylic acid, 3-Carboxymethoxy-5-isobutylamino-thieno[2,3-b]pyridine-2-carboxylic acid, 6-Bromo-3-carboxymethoxy-thieno[3,2-b]thiophene-2-carboxylic acid, 3-Carboxymethoxy-6-(3-cyclohexylamino-phenyl)-thieno[3,2-b]thiophene-2-carboxylic acid, 6-(3-Acetylamino-phenyl)-3-carboxymethoxy-thieno[3,2-b]thiophene-2-carboxylic acid, 3-Carboxymethoxy-6-[3-(cyclohexyl-methoxycarbonyl-amino)-phenyl]-thieno[3,2-b]thiophene-2-carboxylic acid, 3-Carboxymethoxy-6-{3-[cyclohexyl-(3-methyl-butyryl)-amino]-phenyl}-thieno[3,2-b]thiophene-2-carboxylic acid, 3-Carboxymethoxy-6-[3-(1-cyclohexyl-3-isopropyl-ureido)-phenyl]-thieno[3,2-b]thiophene-2-carboxylic acid, 3-(Carboxymethoxy)thieno[3,2-b]pyridine-2-carboxylic acid, 3-(Carboxymethoxy)-6-(trifluoromethyl)thieno[3,2-b]pyridine-2-carboxylic acid, and 3-(Carboxymethoxy)thieno[3,2-b]thiophene-2-carboxylic acid.

These and other compounds of formula (I) were prepared according to the following detailed experimental procedures from commercially available starting materials, intermediates prepared using literature procedures, or novel intermediates described in the schemes and experimental procedures.

### EXAMPLE 1

### 3-Carboxymethoxy-6-methyl-thieno[2,3-b]pyridine-2-carboxylic acid

The first step of Scheme 1: Mercapto-acetic acid methyl ester (0.24 mL, 2.68 mmole) and sodium methoxide (362 mg, 10.72 mmole) were dissolved in 20 mL DMF. The mixture was stirred at room temperature for 5 minutes. 2-Chloro-6-methyl-nicotinic acid methyl ester (0.5 g, 2.68 mmole) in 5 mL of DMF was then added. The resulting solution was stirred at room temperature for 1 hour. DMF was removed under reduced pressure. CH₂Cl₂ (30 mL) was added and the organic layer was washed with diluted HCl once, saturated NaHCO₃ once and brine once. The solution was dried over anhydrous Na₂SO₄ and filtered. The crude product was purified by chromatography on silica gel using a gradient of hexane/EtOAc (0 to 30%) as eluent. Pure fractions were combined and evaporated to give 173 mg (29%) 3-hydroxy-6-methyl-thieno[2,3-b]pyridine-2-carboxylic acid methyl ester as a white solid.

¹H NMR (400 MHz, CHLOROFORM-D) δ ppm 2.70 (s, 3H) 3.96 (s, 3H) 7.22 (d, J=8.34 Hz, 1H) 8.08 (d, 1H) 10.19 (s, 1H).

The second step of Scheme 1: 3-Hydroxy-6-methyl-thieno[2,3-b]pyridine-2-carboxylic acid methyl ester (173 mg, 0.77 mmole) and sodium hydride (62 mg, 1.54 mmole) in 20 mL DMF was stirred at room temperature under nitrogen for 5 minutes. Bromo-acetic acid tert-butyl ester (0.156 mL, 1.16 mmole) was then added and the mixture was stirred at 60° C. for 2 hours. DMF was removed under reduced pressure. CH₂Cl₂ (20 mL) was added and the organic layer was washed with diluted water twice and brine once. The solution was dried over anhydrous Na₂SO₄ and filtered. The crude product was purified by chromatography on silica gel using a gradient of hexane/EtOAc (0 to 20%) as eluent. Pure fractions were combined and evaporated to give 210 mg (81%) 3-tert-butoxycarbonylmethoxy-6-methyl-thieno[2,3-b]pyridine-2-carboxylic acid methyl ester as a white solid.

¹H NMR (400 MHz, CHLOROFORM-D) δ ppm 1.45 (s, 9H) 2.68 (s, 3H) 3.91 (s, 3H) 4.93 (s, 2H) 7.22 (d, J=8.34 Hz, 1H) 8.26 (d, J=8.34 Hz, 1H).

The third step of Scheme 1: 3-tert-Butoxycarbonylmethoxy-6-methyl-thieno[2,3-b]pyridine-2-carboxylic acid methyl ester (210 mg, 0.622 mmole) was dissolved in 5 mL THF. LiOH (78 mg, 1.87 mmole) in 5 mL water was then added. The mixture was stirred at 50° C. for 3 hours. THF was evaporated under reduced pressure. 1N HCl was added slowly and a white precipitate was formed. The precipitate was filtered and washed with water 3 times. 3-Carboxymethoxy-6-methyl-thieno[2,3-b]pyridine-2-carboxylic acid (78 mg, 47%) was obtained as a white solid after drying in a vacuum oven overnight.

¹H NMR (400 MHz, DMSO-D6) 8 ppm 2.62 (s, 3H) 5.01 (d, 2H) 7.41 (d, J=8.34 Hz, 1H) 8.22 (d, J=8.34 Hz, 1H).

### EXAMPLE 2

### 3-Carboxymethoxy-thieno[2,3-b]pyridine-2-carboxylic acid methyl ester

3-tert-Butoxycarbonylmethoxy-thieno[2,3-b]pyridine-2-carboxylic acid methyl ester (155 mg, 70%) was prepared according to the procedures in the second step of Scheme 1 of Example 1.

¹H NMR (400 MHz, CHLOROFORM-D) δ ppm 3.96 (m, 3H) 5.02 (s, 2H) 7.45 (dd, J=8.21, 4.67 Hz, 1H) 8.44 (dd, J=8.08, 1.52 Hz, 1H) 8.69 (dd, J=4.55, 1.52 Hz, 1H).

The first step of Scheme 2: 3-tert-Butoxycarbonylmethoxy-thieno[2,3-b]pyridine-2-carboxylic acid methyl ester (35 mg, 0.11 mmole) was dissolved in 10 mL of TFA/CH₂Cl₂ (v/v 1:1). The solution was stirred at room temperature for 2 hours. Solvents were evaporated. CH₂Cl₂ was added and evaporated (this was repeated 4 times). 3-Carboxymethoxy-thieno[2,3-b]pyridine-2-carboxylic acid methyl ester was obtained as a white solid (>95%).

¹H NMR (400 MHz, CHLOROFORM-D) δ ppm 3.96 (m, 3H) 5.02 (s, 2H) 7.45 (dd, J=8.21, 4.67 Hz, 1H) 8.44 (dd, J=8.08, 1.52 Hz, 1H) 8.69 (dd, J=4.55, 1.52 Hz, 1H).

### EXAMPLE 3

### 3-tert-Butoxycarbonylmethoxy-4-chloro-6-methyl-thieno[3,2-c]pyridine-2-carboxylic acid methyl ester

The first step of Scheme 3: Mercapto-acetic acid methyl ester (0.19 mL, 2.14 mmole) and sodium methoxide (289 mg, 5.35 mmole) were dissolved in 40 mL DMF. The mixture was stirred at room temperature for 5 minutes. 2,4-Dichloro-6-methyl-nicotinic acid ethyl ester (0.5 g, 2.14 mmole) in 10 mL of DMF was then added. The mixture was stirred at room temperature for 2 hours. Bromo-acetic acid tert-butyl ester (0.43 mL, 3.21 mmole) was then added and the mixture was stirred at 70° C. for 16 hours. DMF was removed under reduced pressure. CH₂Cl₂ (20 mL) was added and the organic layer was washed with water three times, saturated NaHCO₃ once and brine once. The solution was dried over anhydrous Na₂SO₄ and filtered. The crude product was purified by chromatography on silica gel using a gradient of hexane/EtOAc (0 to 20%) as eluent. The pure fractions were combined and evaporated to give 404 mg (51%) 3-tert-butoxycarbonylmethoxy-4-chloro-6-methyl-thieno[3,2-c]pyridine-2-carboxylic acid methyl ester as a yellowish green solid.

¹H NMR (400 MHz, CHLOROFORM-D) δ ppm 1.47 (s, 9H) 2.63 (s, 3H) 3.92 (s, 3H) 4.86 (s, 2H) 7.44 (s, 1H).

ESI-MS: m/e=372.18 [M+H]⁺.

### EXAMPLE 4

### 4-Chloro-6-methyl-2-methylcarbamoyl-thieno[3,2-c]pyridin-3-yloxy)-acetic acid tert-butyl ester

The second step of Scheme 3: 3-tert-Butoxycarbonylmethoxy-4-chloro-6-methyl-thieno[3,2-c]pyridine-2-carboxylic acid methyl ester (140 mg, 0.377 mmole) was dissolved in 5 mL of 2M methylamine in THF. The solution was stirred at room temperature for 16 hours. The solvents were removed under reduced pressure. The crude product was purified by chromatography on silica gel using a gradient of hexane/EtOAc (0 to 10%) as eluent. Pure fractions were combined and evaporated to give 76 mg (54%) (4-chloro-6-methyl-2-methylcarbamoyl-thieno[3,2-c]pyridin-3-yloxy)-acetic acid tert-butyl ester.

¹H NMR (400 MHz, CHLOROFORM-D) δ ppm 1.53 (s, 9H) 2.62 (s, 3H) 3.02 (s, 3H) 4.68 (s, 1H) 7.48 (s, 1H) 8.45 (s, 1H).

ESI-MS: m/e=371.20 [M+H]⁺.

### EXAMPLE 5

### (4-Chloro-6-methyl-2-methylcarbamoyl-thieno[3,2-c]pyridin-3-yloxy)-acetic acid

The third step of Scheme 3: (4-Chloro-6-methyl-2-methylcarbamoyl-thieno[3,2-c]pyridin-3-yloxy)-acetic acid (37 mg, 73%) was prepared as a white solid, following the procedure in the first step of Scheme 2 of Example 2.

¹H NMR (400 MHz, CHLOROFORM-D) δ ppm 2.63 (s, 3H) 3.01 (s, 3H) 4.81 (s, 2H) 7.56 (s, 1H).

ESI-MS: m/e=315.12 [M+H]⁺.

### EXAMPLE 6

### 5-Acetylamino-3-carboxymethoxy-thieno[2,3-b]pyridine-2-carboxylic acid

The first step of Scheme 4: A 50 mL concentrated H₂SO₄ solution of 2-hydroxynicotinic acid (6.4 g, 46 mmole) was cooled to 0° C. and concentrated nitric acid (3 mL) was added dropwise. The reaction mixture was then heated to 50° C. overnight. It was then cooled to room temperature and poured onto crushed ice, filtered and washed with water. The resulting solid was recrystallized from 80 mL water. 3.7 g (44%) 5-nitro-2-hydroxynicotinic acid was obtained as a pale yellow solid.

¹H NMR (400 MHz, DMSO-D6) δ ppm 8.72 (d, J=3.28 Hz, 1H) 9.01 (d, J=3.28 Hz, 1H).

The second step of Scheme 4: A 22 mL thionyl chloride solution of 5-nitro-2-hydroxynicotinic acid (3.68 g, 20 mmole) was refluxed for 4 hours. The thionyl chloride was removed under reduced pressure and azeotroped twice with toluene. The resulting oil was cooled in an ice bath and 75 mL MeOH added and stirred for 2 hours at 0° C., then filtered and washed with cold MeOH to give 5-nitro-2-hydroxynicotinic acid methyl ester in quantitative yield as an off-white solid.

¹H NMR (400 MHz, DMSO-D6) δ ppm 3.80 (s, 3H) 8.62 (d, J=3.28 Hz, 1H) 8.87 (d, J=3.28 Hz, 1H).

The third step of Scheme 4: To a 25 mL thionyl chloride suspension of 5-nitro-2-hydroxynicotinic acid methyl ester (4.62 g, 23.3 mmole) was added 1 mL DMF and the mixture heated to reflux until the reaction was complete as judged by TLC. The thionyl chloride was evaporated under reduced pressure and azeotroped twice with toluene. The resulting yellow semi-solid was cooled in an ice bath and treated with 50 mL MeOH. It was stirred for 20 minutes and then poured into 200 mL water, extracted with EtOAc (2×150 mL) and the combined organics washed with dilute aqueous NaOH, water, brine and dried over MgSO₄. 5.03 g (99%) of 2-chloro-5-nitronicotinic acid methyl ester was obtained as a yellow oil.

¹H NMR (400 MHz, CHLOROFORM-D) 8 ppm 4.04 (s, 3H) 8.94 (d, J=2.78 Hz, 1H) 9.33 (d, J=2.78 Hz, 1H).

The fourth step of Scheme 4: To a 5 mL DMF solution of 2-chloro-5-nitronicotinic acid methyl ester (269 mg, 1.25 mmole) was added methyl thioglycolate (111 μL, 1.25 mmole) followed by potassium carbonate (428 mg, 3.1 mmole) and the mixture was stirred at room temperature overnight. The reaction mixture was then poured into 50 mL water and extracted with CH₂Cl₂. The aqueous phase was acidified with 2N HCl and extracted with CH₂Cl₂. Combined organic phases were washed with water and dried over MgSO₄. The solids were removed by filtration and the solvent was evaporated under reduced pressure to give 240 mg (76%) 3-hydroxy-5-nitro-thieno[2,3-b]pyridine-2-carboxylic acid methyl ester as a tan colored solid.

¹H NMR (400 MHz, CHLOROFORM-D) δ ppm 4.02 (s, 3H) 9.01 (d, J=2.53 Hz, 1H) 9.52 (d, J=2.53 Hz, 1H) 10.19 (s, 1H).

The fifth step of Scheme 4: To a 5 mL DMF solution of 3-hydroxy-5-nitro-thieno[2,3-b]pyridine-2-carboxylic acid methyl ester (254 mg, 1.0 mmole) was added potassium carbonate (207 mg, 1.5 mmole) followed by tert-butyl bromoacetate (177 μL, 1.2 mmole). The reaction mixture was heated to 80° C. for 4 hours, then cooled to room temperature, diluted with water, acidified with 1N HCl and extracted with EtOAc. The organic layer was washed with water, brine, dried over (MgSO₄), then filtered and evaporated to obtain the crude product which was then purified by chromatography on silica gel using a gradient of hexane/EtOAc (0 to 10%) as eluent. Pure fractions were combined and evaporated to give 273 mg (74%) 3-tert-butoxycarbonylmethoxy-5-nitro-thieno[2,3-b]pyridine-2-carboxylic acid methyl ester as a white solid.

¹H NMR (400 MHz, CHLOROFORM-D) δ ppm 1.45 (s, 9H) 3.96 (s, 3H) 5.04 (s, 2H) 9.17 (d, J=2.53 Hz, 1H) 9.51 (d, J=2.53 Hz, 1H).

The sixth step of Scheme 4: To a 105 mL suspension of 3-tert-butoxycarbonylmethoxy-5-nitro-thieno[2,3-b]pyridine-2-carboxylic acid methyl ester (1.93 g, 5.7 mmole) was added 35 mL of a saturated solution of copper (II) acetate. NaBH₄ (2.16 g, 57.0 mmole) was added in small portions over several minutes with vigorous, exothermic evolution of N₂. After addition was complete, it was stirred another 30 minutes at room temperature, filtered through a pad of celite and 400 mL CH₂Cl₂ were added. It was then washed with saturated sodium bicarbonate solution and the aqueous phase back-extracted with 100 mL CH₂Cl₂. Combined organic phases were washed with saturated sodium bicarbonate solution and dried (Na₂SO₄). 1.21 g (63%) 5-amino-3-tert-butoxycarbonylmethoxy-thieno[2,3-b]pyridine-2-carboxylic acid methyl ester was obtained as a yellow solid.

¹H NMR (400 MHz, CHLOROFORM-D) δ ppm 1.47 (s, 9H) 3.91 (s, 3H) 4.87 (s, 2H) 7.60 (d, J=2.78 Hz, 1H) 8.26 (d, J=2.78 Hz, 1H).

The seventh step of Scheme 4: To a 2 mL pyridine solution of 5-amino-3-tert-butoxycarbonylmethoxy-thieno[2,3-b]pyridine-2-carboxylic acid methyl ester (45 mg, 0.13 mmole) was added acetic anhydride (17 μL, 0.2 mmole) and the reaction mixture was stirred one hour at room temperature. 20 mL 2N HCl was added and the resulting suspension was extracted with EtOAc. The organic layer was washed with water, brine and dried over (MgSO₄), then filtered and evaporated to obtain the crude product which was then purified by chromatography on silica gel using a gradient of CH₂Cl₂/MeOH (0 to 5%) as eluent. Pure fractions were combined and evaporated to give 22 mg (34%) 5-acetylamino-3-tert-butoxycarbonylmethoxy-thieno[2,3-b]pyridine-2-carboxylic acid methyl ester as a pale yellow oil.

¹H NMR (400 MHz, CHLOROFORM-D) δ ppm 1.47 (s, 9H) 2.23 (s, 3H) 3.91 (s, 3H) 4.94 (s, 2H) 8.03 (s, 1H) 8.50 (d, J=2.53 Hz, 1H) 8.75 (d, J=2.53 Hz, 1H) The eighth step of Scheme 4: 5-Acetylamino-3-tert-butoxycarbonylmethoxy-thieno[2,3-b]pyridine-2-carboxylic acid methyl ester (20 mg, 0.053 mmole) was dissolved in 1.5 mL of a 2:1 mixture of THF:water. LiOH (6 mg, 0.13 mmole) was added and the reaction mixture was stirred at room temperature overnight. The solvents were evaporated and the residue dissolved in 3-4 mL water and acidified dropwise with 1N HCl while stirring. A solid emerged that was filtered, washed with water and vacuum oven dried. 14 mg (85%) 5-acetylamino-3-carboxymethoxy-thieno[2,3-b]pyridine-2-carboxylic acid was obtained as a tan colored solid.

¹H NMR (400 MHz, DMSO-D6) δ ppm 2.12 (s, 3H) 5.03 (s, 2H) 8.65 (d, J=2.53 Hz, 1H) 8.81 (d, J=2.53 Hz, 1H) 10.42 (s, 1H).

### EXAMPLE 7

### 3-Carboxymethoxy-5-nitro-thieno[2,3-b]pyridine-2-carboxylic acid

The eighth step of Scheme 4: 3-Carboxymethoxy-5-nitro-thieno[2,3-b]pyridine-2-carboxylic acid was prepared in 77% yield by hydrolyzing 3-tert-butoxycarbonylmethoxy-5-nitro-thieno[2,3-b]pyridine-2-carboxylic acid methyl ester according to the procedure in Example 6.

¹H NMR (400 MHz, Solvent) δ ppm 5.06 (s, 2H) 9.10 (d, J=2.53 Hz, 1H) 9.38 (d, J=2.53 Hz, 1H).

ESI-MS: m/e=297 [M−H]⁻.

### EXAMPLE 8

### 5-Benzylamino-3-carboxymethoxy-thieno[2,3-b]pyridine-2-carboxylic acid

The seventh step of Scheme 4: To a 1.5 mL DCE solution of 5-amino-3-tert-butoxycarbonylmethoxy-thieno[2,3-b]pyridine-2-carboxylic acid methyl ester (62 mg, 0.2 mmole) was added benzaldehyde (21 mg, 0.2 mmole) followed by acetic acid (17 μL, 0.3 mmole) and sodium triacetoxyborohydride (64 mg, 0.3 mmole) and the resulting mixture was stirred at room temperature overnight. It was diluted with CH₂Cl₂, washed with saturated sodium bicarbonate solution, and dried (MgSO₄). The crude product was purified by preparative thin-layer chromatography (4% EtOAc/CH₂Cl₂). 15 mg (21%) 5-Benzylamino-3-methoxycarbonylmethoxy-thieno[2,3-b]pyridine-2-carboxylic acid methyl ester was obtained as a yellow solid.

¹H NMR (400 MHz, CHLOROFORM-D) δ ppm 3.75 (s, 3H) 3.90 (s, 3H) 4.40 (s, 2H) 4.96 (s, 2H) 7.35 (m, 5H) 7.44 (d, J=3.03 Hz, 1H) 8.24 (d, J=2.53 Hz, 1H).

The eighth step of Scheme 4: 5-Benzylamino-3-methoxycarbonylmethoxy-thieno[2,3-b]pyridine-2-carboxylic acid methyl ester was hydrolyzed following the procedure in the eighth step of Scheme 4 of Example 6 to give 5-benzylamino-3-carboxymethoxy-thieno[2,3-b]pyridine-2-carboxylic acid in 74% yield as a yellow solid.

¹H NMR (400 MHz, DMSO-D6) δ ppm 4.34 (s, 2H) 4.87 (s, 2H) 7.33 (m, 6H) 8.30 (d, J=2.53 Hz, 1H). ESI-MS: m/e=359 [M+H]⁺.

### EXAMPLE 9

### 3-Carboxymethoxy-5-diethylamino-thieno[2,3-b]pyridine-2-carboxylic acid

The seventh step of Scheme 4: 5-Diethylamino-3-methoxycarbonylmethoxy-thieno[2,3-b]pyridine-2-carboxylic acid methyl ester was obtained in 27% yield as a yellow solid, following the procedure in Example 8.

¹H NMR (400 MHz, CHLOROFORM-D) δ ppm 1.22 (t, J=7.07 Hz, 6H) 3.44 (q, J=7.07 Hz, 4H) 3.78 (s, 3H) 3.90 (s, 3H) 4.99 (s, 2H) 7.48 (d, J=3.03 Hz, 1H) 8.33 (d, J=2.78 Hz, 1H).

The eighth step of Scheme 4: 5-Diethylamino-3-methoxycarbonylmethoxy-thieno[2,3-b]pyridine-2-carboxylic acid methyl ester was hydrolyzed following the procedure in Example 6 to give 3-carboxymethoxy-5-diethylamino-thieno[2,3-b]pyridine-2-carboxylic acid in 74% yield as a yellow solid.

¹H NMR (400 MHz, MeOD) δ ppm 1.11 (t, J=7.07 Hz, 6H) 3.39 (q, J=7.07 Hz, 4H) 4.88 (s, 2H) 7.50 (d, J=2.53 Hz, 1H) 8.22 (s, 1H).

ESI-MS: m/e=325 [M+H]⁺.

### EXAMPLE 10

### 6-Benzyloxy-3-carboxymethoxy-thieno[2,3-b]pyridine-2-carboxylic acid

The first step of Scheme 5: A dry round bottom flask was charged with NaH (264 mg of a 60% dispersion in mineral oil, 6.6 mmole) and the reagent washed twice with hexane. 5 mL dry DMF was added and benzyl alcohol (0.62 mL, 6.0 mmole) was added dropwise. After H₂ evolution had ceased the reaction mixture was added dropwise to a 0° C. solution (10 mL) of 2,6-dichloro-nicotinic acid ethyl ester (1.33 g, 6.0 mmole). It was stirred 15 minutes, and then allowed to stir another 20 minutes at room temperature. The reaction mixture was then poured into 150 mL water and extracted with ether (3×75 mL). Combined organic phases were washed with water, brine and dried over MgSO₄. Filtration and evaporation gave the crude product which was then purified by chromatography on silica gel using a gradient of hexane/EtOAc (0 to 4%) as eluent. Pure fractions were combined and evaporated to give 818 mg (47%) of a 1:1 mixture of 2-benzyloxy-6-chloro-nicotinic acid ethyl ester and 6-benzyloxy-2-chloro-nicotinic acid ethyl ester.

The second step of Scheme 5: To a 10 mL EtOH solution of methyl thioglycolate (0.25 mL, 2.8 mmole) was added sodium methoxide (333 mg, 6.2 mmole). After dissolution was complete, 2-benzyloxy-6-chloro-nicotinic acid ethyl ester (818 mg, 2.8 mmole) was added as a solution in EtOH (5 mL) to the mixture. The mixture was heated to reflux for 4 hours, then cooled to room temperature, diluted with water, acidified with 1N HCl, and extracted with EtOAc. The organic phase was then washed with brine and dried over MgSO₄. Filtration and evaporation of the solvent gave the crude product, which was then purified by chromatography on silica gel using a gradient of hexane/CH₂Cl₂ (5 to 20%) as eluent. Pure fractions were combined and evaporated to give 328 mg (36%) 6-benzyloxy-3-hydroxy-thieno[2,3-b]pyridine-2-carboxylic acid ethyl ester as an off-white solid.

¹H NMR (400 MHz, CHLOROFORM-D) δ ppm 1.42 (t, J=7.07 Hz, 3H) 4.41 (q, J=7.07 Hz, 2H) 5.47 (s, 2H) 6.83 (d, J=8.84 Hz, 1H) 7.36 (m, 3H) 7.49 (d, J=6.82 Hz, 2H) 8.03 (d, J=8.59 Hz, 1H) 10.29 (s, 1H).

The third step of Scheme 5: A dry round bottom flask was charged with NaH (43 mg of a 60% dispersion in mineral oil, 1.1 mmole) and the reagent washed twice with hexane. 2 mL of dry DMF was added and 6-benzyloxy-3-hydroxy-thieno[2,3-b]pyridine-2-carboxylic acid ethyl ester (325 mg, 1.0 mmole) was added dropwise as a solution in DMF (3 mL). After H₂ evolution had ceased, tert-butyl bromoacetate (195 mg, 1.0 mmole) was added neat and the reaction mixture was stirred for 3 hours at room temperature. The reaction had not reached completion so the mixture was heated to 60° C. for another 2 hours. It was then cooled to room temperature, diluted with water, and extracted with EtOAc (2×30 mL). The combined organic phases were washed with water, brine and dried over MgSO₄. Filtration and evaporation of the solvent gave the crude product which was then purified by chromatography on silica gel using a gradient of hexane/EtOAc (2 to 8%) as eluent. Pure fractions were combined and concentrated to give 168 mg (38%) 6-benzyloxy-3-tert-butoxycarbonylmethoxy-thieno[2,3-b]pyridine-2-carboxylic acid ethyl ester as a colorless oil, which crystallized on standing.

¹H NMR (400 MHz, CHLOROFORM-D) δ ppm 1.40 (t, J=7.20 Hz, 3H) 1.45 (s, 9 H) 4.36 (q, J=7.07 Hz, 2H) 4.91 (m, 2H) 5.46 (s, 2H) 6.85 (d, J=8.84 Hz, 1H) 7.34 (d, J=7.07 Hz, 1H) 7.39 (t, J=7.20 Hz, 2H) 7.48 (d, J=7.07 Hz, 2H) 8.21 (d, J=8.84 Hz, 1H).

The fourth step of Scheme 5: 6-Benzyloxy-3-tert-butoxycarbonylmethoxy-thieno[2,3-b]pyridine-2-carboxylic acid ethyl ester (56 mg, 0.13 mmol) was dissolved in 1.5 mL of a 2:1 mixture of THF:H₂O and LiOH.H₂O (13 mg, 0.32 mmol) was added and the mixture was stirred at room temperature overnight. The reaction mixture was then evaporated and the residue was redissolved in 2-3 mL of H₂O. The solution was acidified dropwise with 1N HCl while stirring. A solid emerged, which was filtered, washed with water, and vacuum oven-dried to give 36 mg (77%) of 6-benzyloxy-3-carboxymethoxy-thieno[2,3-b]pyridine-2-carboxylic acid as a white solid.

¹H NMR (400 MHz, DMSO-D6) δ ppm 5.00 (s, 2H) 5.45 (s, 2H) 7.03 (d, J=8.84 Hz, 1H) 7.38 (m, 3H) 7.50 (d, J=7.07 Hz, 2H) 8.22 (d, J=8.84 Hz, 1H).

ESI-MS: m/e=358 [M−H]⁻.

### EXAMPLE 11

### 3-Carboxymethoxy-6-carboxymethylsulfanyl-thieno[2,3-b]pyridine-2-carboxylic acid

The first step of Scheme 6: To a 15 mL DMF solution of 2,6-dichloronicotinic acid ethyl ester (663 mg, 3.0 mmole) was added potassium carbonate (2.07 g, 15.0 mmole) followed by methyl thioglycolate (0.56 mL, 6.3 mmole) and the reaction mixture heated to 80° C. overnight. Ethyl bromoacetate (0.40 mL, 3.6 mmole) was then added and the reaction allowed to continue at 80° C. for another 3 hours. It was then cooled to room temperature and diluted with 75 mL water and acidified to pH ˜3 with 1N HCl. The mixture was extracted with EtOAc and the organic layer washed with water, brine and dried over MgSO₄. The crude product was purified by chromatography on silica gel using a gradient of hexane/EtOAc (0 to 25%) as eluent. Pure fractions were combined and evaporated to give 409 mg (32%) 3-ethoxycarbonylmethoxy-6-methoxycarbonylmethylsulfanyl-thieno[2,3-b]pyridine-2-carboxylic acid methyl ester as a pale yellow solid.

¹H NMR (400 MHz, CHLOROFORM-D) δ ppm 1.26 (t, J=7.07, 3H) 3.78 (s, 3H) 3.91 (s, 3H) 4.07 (s, 2H) 4.22 (q, J=7.07 Hz, 2H) 5.01 (s, 2H) 7.24 (d, J=8.59 Hz, 1H) 8.15 (d, J=8.59 Hz, 1H).

The second step of Scheme 6: 3-Ethoxycarbonylmethoxy-6-methoxycarbonylmethylsulfanyl-thieno[2,3-b]pyridine-2-carboxylic acid methyl ester (75 mg, 0.19 mmole) was dissolved in 1 mL of a 3:1:1 mixture of THF:MeOH:H₂O and LiOH.H₂O (27 mg, 0.66 mmole) was added and the reaction mixture allowed to stir at room temperature overnight. The solvents were evaporated and the resulting residue dissolved in 3-4 mL water. The solution was acidified with 1N HCl while stirring and the product precipitated. The solid was filtered, washed with water, and vacuum-oven dried to give 45 mg (72%) of 3-carboxymethoxy-6-carboxymethylsulfanyl-thieno[2,3-b]pyridine-2-carboxylic acid as a white solid.

¹H NMR (400 MHz, DMSO-D6) δ ppm 4.06 (s, 2H) 5.00 (s, 2H) 7.46 (d, J=8.59 Hz, 1H) 8.14 (d, J=8.59 Hz, 1H).

ESI-MS: m/e=344 [M+H]⁺.

### EXAMPLE 12

### 6-Carboxymethanesulfinyl-3-carboxymethoxy-thieno[2,3-b]pyridine-2-carboxylic acid

The first step of Scheme 7: 3-Ethoxycarbonylmethoxy-6-methoxycarbonylmethylsulfanyl-thieno[2,3-b]pyridine-2-carboxylic acid methyl ester (150 mg, 0.38 mg) was dissolved in 1.5 mL CH₂Cl₂ and the solution cooled to 0° C. MCPBA (84 mg of 77% pure reagent, 0.38 mg) was added in one portion. The reaction mixture was stirred one hour at room temperature, diluted with CH₂Cl₂, washed with saturated sodium bicarbonate solution, and dried over MgSO₄. The crude product was purified by chromatography on silica gel using a gradient of hexane/EtOAc (10 to 50%) as eluent. The pure fractions were combined and concentrated to give 127 mg (80%) 3-ethoxycarbonylmethoxy-6-methoxycarbonylmethanesulfinyl-thieno[2,3-b]pyridine-2-carboxylic acid methyl ester as a white solid.

¹H NMR (400 MHz, CHLOROFORM-D) δ ppm 1.27 (t, J=7.20 Hz, 3H) 3.77 (s, 3H) 3.96 (s, 3H) 4.17 (d, J=13.90 Hz, 2H) 4.23 (m, J=7.24, 7.24, 7.24 Hz, 2H) 5.09 (s, 2H) 8.07 (d, J=8.34 Hz, 1H) 8.65 (d, J=8.34 Hz, 1H).

The second step of Scheme 7: 3-Ethoxycarbonylmethoxy-6-methoxycarbonylmethanesulfinyl-thieno[2,3-b]pyridine-2-carboxylic acid methyl ester was hydrolyzed according to the procedure in the second step of Scheme 6 of Example 11 to give 6-carboxymethanesulfinyl-3-carboxymethoxy-thieno[2,3-b]pyridine-2-carboxylic acid in 42% yield as a pale yellow solid.

¹H NMR (400 MHz, MeOD) δ ppm 3.87 (d, J=14.65 Hz, 1H) 4.15 (d, J=14.65 Hz, 1H) 5.01 (s, 2H) 7.92 (d, J=8.59 Hz, 1H) 8.59 (d, J=8.34 Hz, 1H).

### EXAMPLE 13

### 3-Carbamoylmethoxy-thieno[2,3-b]pyridine-2-carboxylic acid

The first step of Scheme 8: A round-bottom flask was charged with NaH (132 mg of 60% dispersion in mineral oil, 3.3 mmole) and washed twice with hexane. 3 mL DMF was then added and methyl thioglycolate (318 mg, 3.0 mmole) was added dropwise as a solution in DMF (4 mL). After hydrogen evolution had ceased, 2-chloronicotinic acid ethyl ester (557 mg, 3.0 mmole) was added dropwise as a solution in DMF (4 mL). The reaction mixture was heated to 65° C. for 90 minutes, cooled to room temperature and diluted with water. The mixture was extracted with ether (2×50 mL) and the combined organic layers were washed with water, brine, and dried over Na₂SO₄. The crude product was purified by flash chromatography on silica gel eluting with a gradient of hexane/EtOAc (2 to 15%). The pure fractions were combined and evaporated to give 390 mg (51%) of 2-methoxycarbonylmethylsulfanyl-nicotinic acid ethyl ester as a white low-melting solid.

¹H NMR (400 MHz, CHLOROFORM-D) δ ppm 1.41 (t, J=7.20 Hz, 3H) 3.73 (s, 3H) 3.93 (s, 2H) 4.41 (q, J=7.24 Hz, 2H) 7.08 (dd, J=7.83, 4.80 Hz, 1H) 8.25 (dd, J=7.83, 1.77 Hz, 1H) 8.52 (dd, J=4.80, 1.77 Hz, 1H).

The second step of Scheme 8: To a 7 mL MeOH solution of 2-methoxycarbonylmethylsulfanyl-nicotinic acid ethyl ester (360 mg, 1.2 mmole) was added sodium methoxide (325 mg, 6.0 mmole) and the mixture was heated to reflux for 30 minutes. The mixture was cooled to room temperature, diluted with water, and acidified with 1N HCl to pH 3. A white solid emerged, which was filtered, washed with water and dried to afford 240 mg (96%) 3-hydroxy-thieno[2,3-b]pyridine-2-carboxylic acid methyl ester.

¹H NMR (400 MHz, CHLOROFORM-D) δ ppm 3.98 (s, 3H) 7.36 (dd, J=8.08, 4.55 Hz, 1H) 8.21 (dd, J=8.08, 1.52 Hz, 1H) 8.72 (dd, J=4.55, 1.52 Hz, 1H) 10.18 (s, 1H).

The third step of Scheme 8: To a 3 mL DMF solution of 3-hydroxy-thieno[2,3-b]pyridine-2-carboxylic acid methyl ester (105 mg, 0.5 mmole) was added potassium carbonate (104 mg, 0.75 mmole) followed by 2-bromoacetamide (108 mg, 0.8 mmole). The reaction mixture was heated to 60° C. for 2 hours. The reaction mixture was cooled and poured into water (35 mL). A solid emerged after several minutes that was filtered and washed with water to obtain 60 mg (45%) 3-carbamoylmethoxy-thieno[2,3-b]pyridine-2-carboxylic acid methyl ester as an off-white solid.

¹H NMR (400 MHz, CHLOROFORM-D) δ ppm 3.95 (s, 3H) 4.86 (s, 2H) 7.41 (dd, J=8.34, 4.55 Hz, 1H) 8.17 (dd, J=8.21, 1.64 Hz, 1H) 8.75 (dd, J=4.55, 1.52 Hz, 1H).

The fourth step of Scheme 8: 3-Carbamoylmethoxy-thieno[2,3-b]pyridine-2-carboxylic acid methyl ester (55 mg, 0.2 mmole) was dissolved in 2 mL of a 3:1:1 mixture of THF:MeOH:H₂O and LiOH.H₂O (10 mg, 0.24 mmole) was added and the reaction mixture was stirred at room temperature for 1 hour. The solvent was evaporated and the resulting residue dissolved in ˜5 mL H₂O and acidified with 1N HCl. A solid emerged that was filtered, washed with water and suction-dried to give 47 mg (93%) of 3-carbamoylmethoxy-thieno[2,3-b]pyridine-2-carboxylic acid as a pale yellow solid.

¹H NMR (400 MHz, DMSO-D6) δ ppm 4.83 (s, 2H) 5.18 (s, 2H) 7.53 (m, 2H) 7.86 (s, 2H) 7.98 (s, 3H) 8.29 (s, 1H) 8.48 (t, J=1.26 Hz, 3H) 8.50 (t, J=1.39 Hz, 3H) 8.69 (dd, J=4.55, 1.52 Hz, 1H) 8.75 (dd, J=4.55, 1.52 Hz, 1H).

ESI-MS: m/e=251 [M−H]⁻.

### EXAMPLE 14

### 3-Carboxymethoxy-6-phenyl-thieno[2,3-b]pyridine-2-carboxylic acid

The first step of Scheme 9: A pressure tube was charged with 2,6-dichloronicotinic acid ethyl ester (221 mg, 1.0 mmole), phenylboronic acid (134 mg, 1.1 mmole), potassium carbonate (346 mg, 2.5 mmole) and Pd(PPh₃)₄ (29 mg, 2.5 mol %) and 1 mL DME added followed by 1.5 mL water. The tube was capped and heated to 60° C. overnight. The reaction mixture was then cooled to room temperature and diluted with ether. The organic phase was washed with 1N NaOH, water, brine and dried over MgSO₄. Filtration and evaporation gave the crude product which was purified by preparative thin layer chromatography (5% EtOAc/Hex). The product band was isolated to give 199 mg of a colorless oil that solidified on standing. NMR analysis showed a 6:1 mixture of 2-chloro-6-phenyl-nicotinic acid ethyl ester:2,6-diphenylnicotinic acid ethyl ester. The solid was recrystallized from hexane to give 90 mg (34%) of 2-chloro-6-phenylnicotinic acid ethyl ester as a white crystalline solid.

¹H NMR (400 MHz, CHLOROFORM-D) δ ppm 1.43 (t, J=7.07 Hz, 3H) 4.44 (q, J=7.07 Hz, 2H) 7.49 (m, 3H) 7.73 (d, J=8.08 Hz, 1H) 8.05 (m, 2H) 8.24 (d, J=8.08 Hz, 1H). The structure was also confirmed by NOE analysis.

The second step of Scheme 9: To a 1 mL DMF solution of methyl thioglycolate (29 μL, 0.32 mmole) was added sodium tert-butoxide (34 mg, 0.35 mmole) and the mixture was stirred at room temperature for 15 minutes. This thiolate solution was then added to a 1 mL DMF solution of 2-chloro-6-phenylnicotinic acid ethyl ester (85 mg, 0.32 mmole) and heated to 60° C. for 1 hour. Another 34 mg of sodium tert-butoxide was added and allowed to stir another 2 hours at 60° C. Ethyl bromoacetate (43 μL, 0.38 mmole) was then added and stirred at the same temperature overnight. The reaction mixture was then cooled to room temperature, diluted with water, acidified with 1N HCl, and extracted with ether. The organic phase was washed with water, brine and dried over MgSO₄. Filtration and evaporation gave the crude product which was purified by preparative thin layer chromatography (CH₂Cl₂). The product band was isolated to give 16 mg (13%) 3-ethoxycarbonylmethoxy-6-phenyl-thieno[2,3-b]pyridine-2-carboxylic acid methyl ester.

¹H NMR (400 MHz, CHLOROFORM-D) δ ppm 1.28 (t, J=7.07 Hz, 3H) 3.94 (s, 3H) 4.24 (q, J=7.24 Hz, 2H) 5.06 (s, 2H) 7.50 (m, 3H) 7.83 (d, J=8.59 Hz, 1H) 8.10 (m, 2H) 8.43 (d, J=8.34 Hz, 1H).

The third step of Scheme 9: 3-Ethoxycarbonylmethoxy-6-phenyl-thieno[2,3-b]pyridine-2-carboxylic acid methyl ester (16 mg, 4.3×10⁻⁵ mmol) was dissolved in 1.5 mL of a 2:1 mixture of THF:H₂O and LiOH.H₂O (6 mg, 0.13 mmol) was added and the mixture was stirred at room temperature overnight. The reaction mixture was then evaporated and the residue redissolved in 2-3 mL of H₂O. It was then acidified dropwise with 1N HCl while stirring. A solid emerged that was filtered, washed with water, and vacuum oven-dried to give 12 mg (85%) of 3-carboxymethoxy-6-phenyl-thieno[2,3-b]pyridine-2-carboxylic acid as a pale yellow solid.

¹H NMR (400 MHz, MeOD) δ ppm 4.98 (s, 2H) 7.41 (m, 3H) 7.87 (d, J=8.59 Hz, 1H) 8.03 (m, 2H) 8.36 (d, J=8.59 Hz, 1H).

ESI-MS: m/e=328 [M−H]⁻.

### EXAMPLE 15

### (2-Cyano-thieno[2,3-b]pyridin-3-yloxy)-acetic acid

The first step of Scheme 10: To a 40 mL DMF solution of 2-mercaptonicotinic acid (1.55 g, 10 mmole) was added potassium tert-butoxide (2.47 g, 22.0 mmole). The mixture was stirred for 15 minutes. After 15 minutes, the mixture became a white suspension. Bromoacetonitrile (0.84 mL, 12.0 mmole) was added and the reaction mixture was stirred 3.5 hours at room temperature. Methyl iodide (0.74 mL, 12.) mmole) was added and the mixture was stirred at room temperature overnight. The reaction mixture was diluted with 100 mL water and acidified with 1N HCl, extracted with CH₂Cl₂ (2×100 mL), the combined organics were washed with dilute NaOH, water and dried over MgSO₄. The mixture was filtered and evaporated to obtain crude product. The mixture was triturated with 10:1 hexane:acetone, filtered, and washed with hexane to give 878 mg (42%) of 2-cyanomethylsulfanyl-nicotinic acid methyl ester.

¹H NMR (400 MHz, DMSO-D6) δ ppm 3.90 (s, 3H) 4.18 (s, 2H) 7.42 (dd, J=7.83, 4.80 Hz, 1H) 8.36 (dd, J=7.83, 1.77 Hz, 1H) 8.78 (dd, J=4.80, 1.77 Hz, 1H).

The second step of Scheme 10: To a 5 mL DMF solution of 2-cyanomethylsulfanyl-nicotinic acid methyl ester (208 mg, 1.0 mmole) was added potassium tert-butoxide (281 mg, 2.5 mmole) and the reaction mixture heated to 60° C. for 30 minutes. Ethyl bromoacetate (665 μL, 6.0 mmole) was added and the reaction mixture was stirred at the same temperature for 2 hours. The mixture was cooled to room temperature, diluted with water and extracted with EtOAc (2×25 mL). Combined organic phases were washed with dilute NaOH, water, brine, and dried over MgSO₄. The crude product was triturated with 3:1 hexane:ether, then filtered and washed with hexane to give 144 mg (55%) (2-cyano-thieno[2,3-b]pyridin-3-yloxy)-acetic acid ethyl ester as a tan colored solid.

¹H NMR (400 MHz, CHLOROFORM-D) δ ppm 1.34 (t, J=7.20 Hz, 3H) 4.33 (q, J=7.16 Hz, 2H) 5.21 (s, 2H) 7.41 (dd, J=8.21, 4.67 Hz, 1H) 8.23 (dd, J=8.21, 1.64 Hz, 1H) 8.75 (dd, J=4.55, 1.77 Hz, 1H).

The third step of Scheme 9: (2-Cyano-thieno[2,3-b]pyridin-3-yloxy)-acetic acid ethyl ester (40 mg, 0.15 mmol) was dissolved in 1.5 mL of a 2:1 mixture of THF:H₂O. LiOH.H₂O (16 mg, 0.38 mmol) was added and the mixture was stirred at room temperature one hour. The reaction mixture was then evaporated and the residue redissolved in 2-3 mL of H₂O. It was then acidified dropwise with 1N HCl while stirring. A solid emerged that was filtered, washed with water, and vacuum oven-dried to give 28 mg (80%) of (2-cyano-thieno[2,3-b]pyridin-3-yloxy)-acetic acid as an off-white solid.

¹H NMR (400 MHz, DMSO-D6) δ ppm 5.29 (s, 2H) 7.62 (dd, J=8.21, 4.67 Hz, 1H) 8.34 (dd, J=8.21, 1.39 Hz, 1H) 8.85 (dd, J=4.67, 1.39 Hz, 1H) 13.53 (s, 1H).

ESI-MS: m/e=233 [M−H]⁻.

### EXAMPLE 16

### [2-(2H-Tetrazol-5-yl)-thieno[2,3-b]pyridin-3-yloxy]-acetic acid

The third step of Scheme 10: (2-Cyano-thieno[2,3-b]pyridin-3-yloxy)-acetic acid ethyl ester (86 mg, 0.33 mmole) was suspended in 1 mL water. Sodium azide (24 mg, 0.36 mmole) and zinc chloride (45 mg, 0.33 mmole) were added and the reaction mixture was heated to reflux overnight. It was then cooled to room temperature, diluted with water, acidified with 2N HCl, and extracted with 5% MeOH/CH₂Cl₂ (3×30 mL). The organic phases were discarded and the aqueous phase, which had a flocculent solid suspended in it, was filtered, washed with water, and vacuum-oven dried. 48 mg (53%) of [2-(2H-tetrazol-5-yl)-thieno[2,3-b]pyridin-3-yloxy]-acetic acid were obtained as a tan-colored solid.

¹H NMR (400 MHz, DMSO-D6) δ ppm 5.03 (s, 2H) 7.56 (m, 1H) 8.48 (d, J=7.83 Hz, 1H) 8.69 (d, J=3.54 Hz, 1H).

ESI-MS: m/e=277 [M−H]⁻.

### EXAMPLE 17

### 3-Carboxymethoxy-6-(isobutylamino-methyl)-thieno[2,3-b]pyridine-2-carboxylic acid hydrochloride

The first step of Scheme 11: To a stirred solution of 2-chloro-6-methyl-nicotinic acid (13.6 g, 80 mmol) in DMF (118 mL) was added potassium carbonate (30 g, 219 mmol) and methyl iodide (23 mL, 366 mmol). After 18 hours, the reaction was diluted with ethyl acetate (300 mL) and washed with water (200 mL). The organic layer was dried over magnesium sulfate and filtered. The solvent was removed under reduced pressure. The material was loaded on silica gel and filtered through a buchner funnel eluting with 1:1 ethyl acetate to yield 14.8 g (99%) of 2-chloro-6-methyl-nicotinic acid methyl ester as a light orange liquid.

¹H NMR (400 MHz, CHLOROFORM-D) δ ppm 2.59 (s, 3H) 3.94 (s, 3H) 7.16 (d, J=7.83 Hz, 1H) 8.09 (d, J=7.83 Hz, 1H).

The second step of Scheme 11: To a stirred solution of 2-chloro-6-methyl-nicotinic acid methyl ester (1.29 g, 6.9 mmol) and methylthioglycolate (618 μL, 6.9 mmol) in DMF (35 mL) at −20° C. was added NaOMe (745 mg, 13.8 mmol) in portions. After 4 hours the solution was diluted with ammonium chloride (200 mL) and extracted with dichloromethane (2×200 mL). The organic layer was dried over magnesium sulfate, filtered, and the solvent was removed under reduced pressure. The material was dissolved in DMF (35 mL) and treated with t-butylbromoacetate (0.93 mL, 6.9 mmol) and sodium methoxide (373 mg, 6.9 mmol). The reaction mixture was stirred for 18 hours at 50° C. The reaction was diluted with ethyl acetate (300 mL), and washed with sodium bicarbonate (100 mL) and brine (100 mL). The organic layer was dried over magnesium sulfate and filtered. The solvent was removed under reduced pressure and the material was purified by CombiFlash chromatography eluting with 5-30% ethyl acetate-hexane gradient to give 757 mg of 3-tert-butoxycarbonylmethoxy-6-methyl-thieno[2,3-b]pyridine-2-carboxylic acid methyl ester as an off white solid (32%).

¹H NMR (400 MHz, CHLOROFORM-D) δ ppm 1.46 (m, 9H) 2.69 (s, 3H) 3.92 (s, 3 H) 4.92 (s, 2H) 7.23 (d, J=8.34 Hz, 1H) 8.27 (d, J=8.34 Hz, 1H).

The third step of Scheme 11: To a solution of 3-tert-butoxycarbonylmethoxy-6-methyl-thieno[2,3-b]pyridine-2-carboxylic acid methyl ester (290 mg, 0.9 mmol) in deoxygenated carbon tetrachloride (5.0 mL) was added N-bromosuccinimide (153 mg, 0.9 mmol) and AIBN (14 mg, 0.09 mmol). The reaction was heated to reflux for 3 hours. To the reaction was added N-bromosuccinimide (53 mg, 0.3 mmol). Three hours later, the reaction was cooled to room temperature. The solvent was removed under reduced pressure and the material was purified by CombiFlash column chromatography eluting with ethyl acetate-dichloromethane (0-5% gradient) to give 128 mg of 6-bromomethyl-3-tert-butoxycarbonylmethoxy-thieno[2,3-b]pyridine-2-carboxylic acid methyl ester as an orange solid (36%).

¹H NMR (400 MHz, CHLOROFORM-D) δ ppm 1.47 (m, 9H) 3.93 (s, 3H) 4.65 (s, 2H) 4.93 (s, 2H) 7.51 (d, J=8.34 Hz, 1H) 8.40 (d, J=8.34 Hz, 1H).

The fourth step of Scheme 11: To a solution of 6-bromomethyl-3-tert-butoxycarbonylmethoxy-thieno[2,3-b]pyridine-2-carboxylic acid methyl ester (22 mg, 0.05 mmol) and triethylamine (22 μL, 0.16 mmol) in THF (1 mL) was added isobutylamine (8 μL, 0.08 mmol). After 18 hours the solvent was removed under reduced pressure. The material was purified by combiflash chromatography eluting with 0-10% methanol-dichloromethane to give 14 mg of 3-tert-butoxycarbonylmethoxy-6-(isobutylamino-methyl)-thieno[2,3-b]pyridine-2-carboxylic acid methyl ester hydrochloride as an off white solid (65%).

¹H NMR (400 MHz, CHLOROFORM-D) δ ppm 0.96 (d, J=6.82 Hz, 6H) 1.45 (s, 9H) 1.86 (m, 1H) 2.53 (d, J=6.82 Hz, 2H) 2.60 (s, 1H) 3.92 (s, 3H) 4.08 (s, 2H) 4.93 (s, 2H) 7.42 (d, J=8.34 Hz, 1H) 8.34 (d, J=8.34 Hz, 1H).

The fifth step of Scheme 11: A solution of 3-tert-butoxycarbonylmethoxy-6-(isobutylamino-methyl)-thieno[2,3-b]pyridine-2-carboxylic acid methyl ester hydrochloride (14 mg, 0.03 mmol) and 0.1 M LiOH (0.7 mL, 0.07 mmol) in 1:1 THF-water (1 mL) was stirred vigorously at room temperature. After 8 hours, the THF was removed under reduced pressure. The reaction was treated with 1N HCl (70 μL, 0.07 mmol) dropwise. A white precipitate was collected, washed with water and dried to give 6 mg of 3-carboxymethoxy-6-(isobutylamino-methyl)-thieno[2,3-b]pyridine-2-carboxylic acid hydrochloride as a white solid (47%).

¹H NMR (400 MHz, DMSO-D6) δ ppm 0.96 (d, J=6.57 Hz, 6H) 2.03 (m, 1H) 2.82 (d, J=6.82 Hz, 2H) 4.44 (s, 2H) 4.76 (s, 2H) 7.55 (d, J=8.34 Hz, 1H) 8.41 (d, J=8.34 Hz, 1H).

### EXAMPLE 18

### 6-(Benzylamino-methyl)-3-carboxymethoxy-thieno[2,3-b]pyridine-2-carboxylic acid hydrochloride

The fourth step of Scheme 11: A solution of 6-bromomethyl-3-tert-butoxycarbonylmethoxy-thieno[2,3-b]pyridine-2-carboxylic acid methyl ester (28 mg, 0.07 mmol) and triethylamine (28 μL, 0.2 mmol) in THF (1 mL) was treated with benzylamine (15 μL, 0.14 mmol) at 0° C. The reaction was warmed to room temperature and then heated to 40° C. for 3 hours. The reaction was cooled to room temperature and the solvent was removed. The material was purified by CombiFlash column chromatography eluting with a 0-10% methanol-dichloromethane gradient to provide 22 mg of 6-(benzylamino-methyl)-3-tert-butoxycarbonylmethoxy-thieno[2,3-b]pyridine-2-carboxylic acid methyl ester hydrochloride as a white solid (76%).

¹H NMR (400 MHz, CHLOROFORM-D) δ ppm 1.45 (s, 9H) 3.92 (d, J=3.79 Hz, 3H) 4.06 (s, 2H) 4.92 (s, 2H) 7.35 (m, 6H) 8.33 (d, J=8.08 Hz, 1H).

The fifth step of Scheme 11: 6-(Benzylamino-methyl)-3-carboxymethoxy-thieno[2,3-b]pyridine-2-carboxylic acid hydrochloride was prepared following the procedure in Example 17 to give 7 mg of 6-(benzylamino-methyl)-3-carboxymethoxy-thieno[2,3-b]pyridine-2-carboxylic acid hydrochloride (29%).

¹H NMR (400 MHz, DMSO-D6) δ ppm 4.23 (s, 2H) 4.43 (s, 2H) 4.79 (s, 2H) 7.43 (m, 3H) 7.53 (m, 3H) 8.39 (d, J=8.34 Hz, 1H).

### EXAMPLE 19

### 3-Carboxymethoxy-6-[(2-methoxy-ethylamino)-methyl]-thieno[2,3-b]pyridine-2-carboxylic acid hydrochloride

The fourth step of Scheme 11: A stirred solution of 6-bromomethyl-3-tert-butoxycarbonylmethoxy-thieno[2,3-b]pyridine-2-carboxylic acid methyl ester (30 mg, 0.07 mmol) and triethylamine (30 μL, 0.22 mmol) in THF (1 mL) was treated with 2-methoxyethylamine (13 μL, 0.14 mmol). After 6 hours, the solvent was removed under reduced pressure. The material was purified by CombiFlash column chromatography eluting with 0-10% methanol-dichloromethane with 2% triethylamine to give 26 mg of 3-tert-butoxycarbonylmethoxy-6-[(2-methoxy-ethylamino)-methyl]-thieno[2,3-b]pyridine-2-carboxylic acid methyl ester hydrochloride as a white solid (86%).

¹H NMR (400 MHz, CHLOROFORM-D) δ ppm 1.45 (m, 9H) 3.04 (m, 2H) 3.40 (s, 3H) 3.66 (m, 2H) 3.93 (s, 3H) 4.23 (s, 1H) 4.93 (s, 2H) 5.30 (s, 2H) 7.42 (d, J=8.34 Hz, 1H) 8.39 (d, J=8.34 Hz, 1H).

The fifth step of Scheme 11: 3-Carboxymethoxy-6-[(2-methoxy-ethylamino)-methyl]-thieno[2,3-b]pyridine-2-carboxylic acid hydrochloride was prepared according to the procedure in Example 17 to provide 25 mg of a white solid (98%).

¹H NMR (400 MHz, DMSO-D6) δ ppm 3.19 (m, 2H) 3.32 (m, 3H) 3.66 (m, 2H) 4.46 (s, 2H) 4.79 (s, 2H) 5.76 (s, 1H) 7.60 (d, J=8.34 Hz, 1H) 8.40 (d, J=8.34 Hz, 1H).

### EXAMPLE 20

### 3-Carboxymethoxy-6-[(2-thiophen-3-yl-ethylamino)-methyl]-thieno[2,3-b]pyridine-2-carboxylic acid hydrochloride

The fourth step of Scheme 11: A stirred solution of 6-bromomethyl-3-tert-butoxycarbonylmethoxy-thieno[2,3-b]pyridine-2-carboxylic acid methyl ester (36 mg, 0.09 mmol) and triethylamine (36 μL, 0.3 mmol) was treated with 2-thiophen-3-yl-ethylamine (22 mg, 0.2 mmol). After 6 hours, the solvent was removed under reduced pressure and the material was purified by CombiFlash column chromatography eluting with 0-10% methanol-dichloromethane; 2% triethylamine to give 28 mg of 3-tert-butoxycarbonylmethoxy-6-[(2-thiophen-3-yl-ethylamino)-methyl]-thieno[2,3-b]pyridine-2-carboxylic acid methyl ester hydrochloride as a white solid (70%).

¹H NMR (400 MHz, CHLOROFORM-D) δ ppm 1.45 (s, 9H) 2.98 (m, 2H) 3.08 (t, J=6.82 Hz, 2H) 3.92 (d, J=2.02 Hz, 3H) 4.07 (s, 2H) 4.92 (s, 2H) 6.86 (d, J=3.54 Hz, 1H) 6.94 (dd, J=5.18, 3.41 Hz, 1H) 7.15 (m, 1H) 7.39 (d, J=8.34 Hz, 1H) 8.32 (d, J=8.34 Hz, 1H).

The fifth step of Scheme 11: 3-Carboxymethoxy-6-[(2-thiophen-3-yl-ethylamino)-methyl]-thieno[2,3-b]pyridine-2-carboxylic acid hydrochloride (15 mg, 61%) was prepared according to the procedure in Example 17.

¹H NMR (400 MHz, DMSO-D6) δ ppm 4.51 (s, 2H) 4.79 (s, 2H) 7.00 (m, 1H) 7.42 (dd, J=5.05, 1.26 Hz, 1H) 7.54 (d, J=8.34 Hz, 1H) 8.41 (d, J=8.34 Hz, 1H).

### EXAMPLE 21

### 6-(Benzoylamino-methyl)-3-carboxyMethoxy-thieno[2,3-b]pyridine-2-carboxylic acid hydrochloride

The fourth step of Scheme 11: A stirred solution of 6-bromomethyl-3-tert-butoxycarbonylmethoxy-thieno[2,3-b]pyridine-2-carboxylic acid methyl ester (32 mg, 0.07 mmol) and triethylamine (42 mL, 0.3 mmol) in dichloromethane (4 mL) was treated with benzoylchloride (17 mL, 0.15 mmol). After 2 h, the solvent was removed under reduced pressure. The material was purified by Combiflash column chromatography eluting with 50-100% ethyl acetate-hexane to give 29 mg of 6-(benzoylamino-methyl)-3-tert-butoxycarbonylmethoxy-thieno[2,3-b]pyridine-2-carboxylic acid methyl ester hydrochloride as a white solid (93%).

¹H NMR (400 MHz, CHLOROFORM-D) δ ppm 1.26 (m, 3H) 3.94 (s, 3H) 4.22 (q, J=7.24 Hz, 2H) 4.92 (d, J=4.80 Hz, 2H) 5.05 (s, 2H) 7.41 (d, J=8.08 Hz, 1H) 7.51 (m, 5H) 7.90 (d, J=7.33 Hz, 1H) 8.38 (d, J=8.34 Hz, 1H).

The fifth step of Scheme 11: 6-(Benzoylamino-methyl)-3-carboxymethoxy-thieno[2,3-b]pyridine-2-carboxylic acid hydrochloride (19 mg, 66%) was prepared according to the procedure in Example 17 as a white solid.

¹H NMR (400 MHz, DMSO-D6) δ ppm 4.70 (d, J=6.06 Hz, 2H) 4.97 (s, 2H) 7.53 (m, 5H) 7.93 (m, 1H) 8.31 (d, J=8.59 Hz, 1H) 9.27 (t, J=5.81 Hz, 1H).

### EXAMPLE 22

### 6-(Benzenesulfonylamino-methyl)-3-carboxymethoxy-thieno[2,3-b]pyridine-2-carboxylic acid

The fourth step of Scheme 11: To a solution of 6-bromomethyl-3-tert-butoxycarbonylmethoxy-thieno[2,3-b]pyridine-2-carboxylic acid methyl ester (32 mg, 0.07 mmol) in 1:1 dichloromethane-aqueous sodium bicarbonate (5 mL) was added phenylsulfonylchloride (19 μL, 0.15 mmol). The reaction was stirred vigorously at room temperature for 2 hours and then diluted with ethyl acetate (100 mL). The organic layer was washed with saturated aqueous sodium bicarbonate (25 mL) and dried over magnesium sulfate. The solution was filtered and the solvent was removed under reduced pressure. The material was purified by CombiFlash column chromatography eluting with 25-85% ethyl acetate-hexane to yield 33 mg of 6-(benzenesulfonylamino-methyl)-3-tert-butoxycarbonylmethoxy-thieno[2,3-b]pyridine-2-carboxylic acid methyl ester hydrochloride as a white solid (99%).

¹H NMR (400 MHz, CHLOROFORM-D) 8 ppm 1.27 (t, J=7.20 Hz, 3H) 1.41 (d, J=6.82 Hz, 6H) 3.19 (m, 1H) 3.94 (s, 3H) 4.22 (q, J=7.07 Hz, 2H) 4.59 (d, J=5.31 Hz, 2H) 5.05 (s, 2H) 5.43 (m, 1H) 7.34 (d, J=8.34 Hz, 1H) 8.39 (d, J=8.34 Hz, 1H).

The fifth step of Scheme 11: 6-(Benzenesulfonylamino-methyl)-3-carboxymethoxy-thieno[2,3-b]pyridine-2-carboxylic acid (66%) was prepared according to the procedure in Example 17 to give a white solid.

¹H NMR (400 MHz, DMSO-D6) δ ppm 4.06 (d, J=6.32 Hz, 2H) 4.80 (s, 2H) 7.36 (d, J=8.34 Hz, 2H) 7.58 (m, 3H) 7.79 (m, 2H) 8.26 (t, J=6.19 Hz, 1H).

### EXAMPLE 23

### 3-Carboxymethoxy-4-chloro-thieno[2,3-c]pyridine-2-carboxylic acid

The first step of Scheme 12: To a solution of methylthioglycolate (131 μL, 1.5 mmol) in N,N-dimethylformamide (3.6 mL) at −30° C. was added sodium hydride (60% wt; 70 mg, 1.8 mmol). The solution was added slowly to a solution of 3,5-dichloroisonicotinic acid methyl ester (300 mg, 1.5 mmol) in DMF (3 mL) at −50° C. The reaction was warmed slowly to room temperature and stirred overnight. The reaction was poured into saturated ammonium chloride (140 mL) and extracted with ethyl acetate (2×100 mL). The organic layer was dried over magnesium sulfate, filtered, and the solvent was removed under reduced pressure. The material was purified by CombiFlash column chromatography eluting with 7:3 dichloromethane-ethyl acetate to give 41 mg (12%) of 4-chloro-3-hydroxy-thieno[2,3-c]pyridine-2-carboxylic acid methyl ester as an off white solid.

¹H NMR (400 MHz, Solvent) δ ppm 3.89 (d, J=2.27 Hz, 3H) 8.34 (s, 1H) 8.91 (s, 1H).

The second step of Scheme 12: To a solution of 4-chloro-3-hydroxy-thieno[2,3-c]pyridine-2-carboxylic acid methyl ester (28 mg, 0.12 mmol) in N,N-dimethylformamide (5 mL) was added sodium hydride (60% wt; 5 mg, 0.13 mmol) at 0° C. After 10 minutes, t-butylbromoacetate (18 μL, 0.12 mmol) was added to the solution. The reaction was warmed to room temperature and stirred for 2 hours. The reaction was poured into saturated sodium bicarbonate (50 mL) and extracted with ethyl acetate (2×25 mL). The organic layer was dried over magnesium sulfate and filtered. The solvent was removed under reduced pressure and the material was purified by CombiFlash column chromatography eluting with a 5-30% ethyl acetate-hexane gradient to give 19 mg of 3-tert-butoxycarbonylmethoxy-4-chloro-thieno[2,3-c]pyridine-2-carboxylic acid methyl ester as a light brown solid.

¹H NMR (400 MHz, MeOD) δ ppm 5.05 (m, 2H) 5.37 (m, 2H) 7.37 (m, 3H) 7.49 (m, 2H) 8.14 (m, 1H) 8.30 (m, 1H).

The third step of Scheme 12: The procedure in the fifth step of Scheme 11 of Example 17 was followed to afford 13 mg of 3-carboxymethoxy-4-chloro-thieno[2,3-c]pyridine-2-carboxylic acid as a white solid.

¹H NMR (400 MHz, DMSO-D6) δ ppm 4.88 (s, 2H) 8.54 (s, 1H) 9.22 (s, 1H).

### EXAMPLE 24

### 5-Bromo-3-carboxymethoxy-thieno[2,3-b]pyridine-2-carboxylic acid

The first step of Scheme 13: NBS (6.84 g, 38.4 mmol, 1.3equiv) was added to a solution of 2-hydroxy-nicotinic acid methyl ester (4.47 g, 29.6 mmol, 1.0 equiv) in 100 mL of CH₂Cl₂ and refluxed for 20 hr. The solvent was extracted with water (3×100 mL), dried over MgSO₄, filtered, and removed by rotary evaporation leaving 6.75 g (98%) of 5-bromo-2-hydroxy-nicotinic acid methyl ester as a light yellow solid. Known compound. J. Org. Chem. 1989, 54, 3618-36-24.

The second step of Scheme 13: 5-Bromo-2-hydroxy-nicotinic acid methyl ester (6.50 g, 28.0 mmol) was stirred in 80 mL of POCl₃ at room temperature for 16 hr then refluxed for 5 hr. After removal of excess POCl₃ by rotary evaporation, DCM was added and the mixture was extracted with water. All organic layers were combined, washed with brine, and dried over MgSO₄, leaving 6.1 g (87%) of 5-bromo-2-chloro-nicotinic acid methyl ester as a red liquid. The properties of this compound are reported in J. Org. Chem. 1989, 54.

The third step of Scheme 13: Potassium carbonate (6.5 g, 47.0 mmol, 2.0 equiv) was added to a mixture of 5-bromo-2-chloro-nicotinic acid methyl ester (5.83 g, 23.3 mmol, 1 equiv) and methyl thioglycolate (2.1 mL, 23.5 mmol, 1 equiv) in DMF (50 mL) and stirred at 50° C. for 7 hr. Water (250 mL) was added and the mixture was acidified with 1N HCl. The solids were filtered and washed with water leaving 5-bromo-3-hydroxy-thieno[2,3-b]pyridine-2-carboxylic acid methyl ester 3.2 g (47%).

¹H NMR (400 MHz, CHLOROFORM-D) δ ppm 3.98 (s, 3H) 8.34 (d, J=2.27 Hz, 1H) 8.74 (d, J=2.27 Hz, 1H) 10.10 (s, 1H).

The fourth step of Scheme 13: Potassium carbonate (1.82 g, 13.2 mmol, 1.5 equiv) was added to a mixture of 5-bromo-3-hydroxy-thieno[2,3-b]pyridine-2-carboxylic acid methyl ester (2.53 g, 8.8 mmol, 1 equiv) and tert-butyl bromoacetate (1.7 mL, 11.4 mmol, 1.3 equiv) in 50 mL DMF and stirred at 50° C. for 15 hr. After adding the mixture to water, it was extracted with Et₂O (3×150 mL). The organic layers were combined and washed with water (4×150 mL), dried over MgSO₄, filtered, and rotary evaporated leaving 3.17 g crude material. Recrystallization using hot hexanes yielded 5-bromo-3-tert-butoxycarbonylmethoxy-thieno[2,3-b]pyridine-2-carboxylic acid methyl ester 1.98 g (56%) as an orange solid.

¹H NMR (400 MHz, CHLOROFORM-D) δ ppm 1.46 (s, 9H) 3.93 (s, 3H) 4.94 (s, 2 H) 8.52 (d, J=2.27 Hz, 1H) 8.73 (d, J=2.27 Hz, 1H).

The fifth step of Scheme 13: A mixture of 5-bromo-3-tert-butoxycarbonylmethoxy-thieno[2,3-b]pyridine-2-carboxylic acid methyl ester (54 mg, 0.13 mmol) and LiOH (17 mg, 3equiv) was stirred in 6 mL of THF/H₂O (1:1) for 5 hr. After rotary evaporation to remove the THF, the mixture was acidified using 1N HCl until a precipitate formed. Filtration followed by washing with water yielded 28 mg (65%) of 5-bromo-3-carboxymethoxy-thieno[2,3-b]pyridine-2-carboxylic acid as an off-white solid.

¹H NMR (400 MHz, DMSO-D6) δ ppm 5.02 (s, 2H) 8.55 (d, J=2.27 Hz, 1H) 8.86 (d, J=2.27 Hz, 1H).

### EXAMPLE 25

### 3-Carboxymethoxy-5-iodo-thieno[2,3-b]pyridine-2-carboxylic acid

3-Carboxymethoxy-5-iodo-thieno[2,3-b]pyridine-2-carboxylic acid was prepared according procedures similar to that for Example 24.

The second step of Scheme 13: 5-Iodo-2-hydroxy-nicotinic acid methyl ester (4.75 g, 17.1 mmol) was refluxed in POCl₃ (50 mL) for 24 hr. The solvent was removed via rotary evaporation. The crude material was neutralized using NaHCO₃ sat, extracted with ether, dried over MgSO₄, filtered, and rotary evaporated. Purification by silica chromatography eluting with 5% EtOAc in hexanes yielded 4.3 g (85%) of 2-chloro-5-iodo-nicotinic acid methyl ester. The properties of this compound are reported in J. Org. Chem. 1989, 54.

The third step of Scheme 13: Sodium methoxide (0.96 g, 47.0 mmol, 2.0 equiv) was added to a mixture 2-chloro-5-iodo-nicotinic acid methyl ester (3.93 g, 13.3 mmol, 1 equiv.) and methyl thioglycolate (1.19 mL, 13.3 mmol, 1 equiv) in DMF (30 mL) and stirred at room temperature for 7 hr. Water was added and the mixture was extracted with EtOAc. The organic layers were combined, washed with water, dried over MgSO₄, filtered, and rotary evaporated. The crude solids were recrystallized in MeOH leaving 3-hydroxy-5-iodo-thieno[2,3-b]pyridine-2-carboxylic acid methyl ester 1.25 g (28%) as a light brown solid.

¹H NMR (400 MHz, DMSO-D6) δ ppm 3.85 (s, 3H) 8.71 (d, J=2.02 Hz, 1H) 8.91 (d, J=2.02 Hz, 1H) 11.10 (s, 1H).

The fourth step of Scheme 13: 3-Hydroxy-5-iodo-thieno[2,3-b]pyridine-2-carboxylic acid methyl ester (444 mg, 1.3 mmol), K₂CO₃ (275 mg, 1.5 equiv), and tert-butyl bromoacetate (0.3 mL, 1.5 equiv) were combined in DMF (8 mL) and stirred at 50° C. for 20 hr. Water (20 mL) was added and the mixture was extracted with DCM. After removal of solvent by rotary evaporation, the crude material was purified by column chromatography eluting with 5% EtOAc in hexanes yielding 403 mg (68%) of 3-tert-butoxycarbonylmethoxy-5-iodo-thieno[2,3-b]pyridine-2-carboxylic acid methyl ester as an off-white solid.

¹H NMR (400 MHz, DMSO-D6) δ ppm 1.40 (s, 9H) 3.87 (s, 3H) 4.98 (s, 2H) 8.69 (d, J=2.27 Hz, 1H) 8.96 (d, J=2.02 Hz, 1H).

The fifth step of Scheme 13: 3-tert-Butoxycarbonylmethoxy-5-iodo-thieno[2,3-b]pyridine-2-carboxylic acid methyl ester (57 mg, 0.13 mmol) was hydrolyzed according to the procedure in fifth step of Scheme 13 of Example 24 to yield 32 mg (65%) of 3-carboxymethoxy-5-iodo-thieno[2,3-b]pyridine-2-carboxylic acid as a white solid.

¹H NMR (400 MHz, DMSO-D6) δ ppm 5.00 (s, 2H) 8.68 (d, J=2.02 Hz, 1H) 8.93 (d, J=2.02 Hz, 1H).

### EXAMPLE 26

### 3-Carboxymethoxy-5-styryl-thieno[2,3-b]pyridine-2-carboxylic acid

5-Bromo-3-tert-butoxycarbonylmethoxy-thieno[2,3-b]pyridine-2-carboxylic acid methyl ester was prepared according to procedures in steps one through four of Scheme 13 of Example 24.

The first step of Scheme 14: 5-Bromo-3-tert-butoxycarbonylmethoxy-thieno[2,3-b]pyridine-2-carboxylic acid methyl ester (59 mg, 0.15 mmol, 1 equiv) was combined with Pd(OAc)₂ (2.2 mg), 2-di-tert-butylphosphino biphenyl (6 mg), potassium fluoride (26 mg), and trans-phenylethenylboronic acid (32 mg, 0.22 mmol, 1.4 equiv). The septum-sealed vessel was vacuumed and purged with nitrogen 3 times and 0.8 mL THF was added and the mixture was stirred at room temperature for 17 hr. The crude mixture was absorbed onto celite and purified by silica column eluting with 5% EtOAc in hexanes yielding 3-tert-butoxycarbonylmethoxy-5-styryl-thieno[2,3-b]pyridine-2-carboxylic acid methyl ester 45 mg (70%) as an off-white solid.

¹H NMR (400 MHz, CHLOROFORM-D) δ ppm 1.47 (s, 9H) 3.92 (s, 3H) 4.97 (s, 2H) 7.29-7.16 (m, 2H) 7.34-7.27 (m, 1H) 7.39 (t, J=7.45 Hz, 2H) 7.55 (d, J=7.33 Hz, 2H). 8.44 (d, J=2.27 Hz, 1H) 8.83 (d, J=2.02 Hz, 1H).

The second step of Scheme 14: 3-tert-Butoxycarbonylmethoxy-5-styryl-thieno[2,3-b]pyridine-2-carboxylic acid methyl ester (45 mg, 0.11 mmol) was hydrolyzed according to the procedure in the fifth step of Scheme 13 of Example 24 to yield 3-carboxymethoxy-5-styryl-thieno[2,3-b]pyridine-2-carboxylic acid 28 mg (72%) as a yellow solid.

¹H NMR (400 MHz, DMSO-D6) δ ppm 5.06 (s, 2H) 7.32 (t, J=7.33 Hz, 1H) 7.42 (t, J=7.58 Hz, 2H) 7.49 (s, 1H) 7.51 (s, 1H) 7.67 (d, J=7.33 Hz, 2H) 8.51 (d, J=2.02 Hz, 1H) 9.02 (d, J=2.27 Hz, 1H).

### EXAMPLE 27

### 3-Carboxymethoxy-5-phenyl-thieno[2,3-b]pyridine-2-carboxylic acid

The first step of Scheme 14: Following the procedure in Example 26, 5-bromo-3-tert-butoxycarbonylmethoxy-thieno[2,3-b]pyridine-2-carboxylic acid methyl ester (52 mg, 0.13 mmol, 1 equiv), Pd(OAc)₂ (2.2 mg), 2-di-tert-butylphosphino biphenyl (6 mg), potassium fluoride (23 mg), and phenyl boronic acid (24 mg, 1.5 equiv) were used. Purification by column chromatography yielded 3-tert-butoxycarbonylmethoxy-5-phenyl-thieno[2,3-b]pyridine-2-carboxylic acid methyl ester 41 mg (79%) as an off-white solid.

¹H NMR (400 MHz, CHLOROFORM-D) δ ppm 1.44 (s, 9H) 3.94 (s, 3H) 4.98 (s, 2H) 7.43 (t, J=7.33 Hz, 1H) 7.51 (t, J=7.45 Hz, 2H) 7.66 (d, J=7.07 Hz, 2H) 8.53 (d, J=2.27 Hz, 1H) 8.94 (d, J=2.27 Hz, 1H).

The second step of Scheme 14: 3-tert-Butoxycarbonylmethoxy-5-phenyl-thieno[2,3-b]pyridine-2-carboxylic acid methyl ester (41 mg, 0.10 mmol) was hydrolyzed according to the procedure in the fifth step of Scheme 13 of Example 24 to yield 3-carboxymethoxy-5-phenyl-thieno[2,3-b]pyridine-2-carboxylic acid, 19 mg (56%).

¹H NMR (400 MHz, DMSO-D6) δ ppm 5.04 (s, 2H) 7.47 (t, J=7.33 Hz, 1H) 7.56 (t, J=7.45 Hz, 2H) 7.82 (d, J=7.07 Hz, 2H) 8.56 (d, J=2.27 Hz, 1H) 9.06 (d, J=2.27 Hz, 1H).

### EXAMPLE 28

### 5-Benzyl-3-carboxymethoxy-thieno[2,3-b]pyridine-2-carboxylic acid

The first step of Scheme 14: Following the procedure in Example 26, 5-bromo-3-tert-butoxycarbonylmethoxy-thieno[2,3-b]pyridine-2-carboxylic acid methyl ester (56 mg, 0.14 mmol, 1 equiv), Pd(OAc)₂ (2.2 mg), 2-di-tert-butylphosphino biphenyl (7 mg), potassium fluoride (24 mg), and benzyl-9BBN 0.5M in THF (0.3 mL, 2.0 equiv) were used. Purification by column chromatography yielded 5-benzyl-3-tert-butoxycarbonylmethoxy-thieno[2,3-b]pyridine-2-carboxylic acid methyl ester 51 mg (88%) as a yellow film.

¹H NMR (400 MHz, CHLOROFORM-D) δ ppm 1.43 (s, 9H) 3.91 (s, 3H) 4.12 (s, 2H) 4.91 (s, 2H) 7.39-7.15 (m, 5H) 8.19 (d, J=2.27 Hz, 1H) 8.58 (d, J=2.02 Hz, 1H).

The second step of Scheme 14: 5-Benzyl-3-tert-butoxycarbonylmethoxy-thieno[2,3-b]pyridine-2-carboxylic acid methyl ester (51 mg, 0.12 mmol) was hydrolyzed according to the procedure in the fifth step of Scheme 13 of Example 24 to yield 5-benzyl-3-carboxymethoxy-thieno[2,3-b]pyridine-2-carboxylic acid 7 mg (17%) as a yellow solid.

¹H NMR (400 MHz, DMSO-D6) δ ppm 4.14 (s, 2H) 4.98 (s, 2H) 7.24-7.15 (m, 2H) 7.33-7.26 (m, 4H) 8.20 (d, J=2.02 Hz, 1H) 8.67 (d, J=2.02 Hz, 1H).

### EXAMPLE 29

### 3-Carboxymethoxy-6-chloro-benzo[b]thiophene-2-carboxylic acid

The second step of Scheme 15: 6-Chloro-3-hydroxy-benzo[b]thiophene-2-carboxylic acid methyl ester (5.28 g, 21.8 mmol, 1 equiv), K₂CO₃ (4.51 g, 1.5 equiv), and tert-butyl bromoacetate (4.2 mL, 1.3equiv) were stirred at 50° C. in DMF (160 mL) for 18 hr. Water was added and the mixture was extracted with ether (3×75 mL). The organic layers were combined, washed with brine, dried over MgSO₄, filtered, rotary evaporated, and vacuumed overnight leaving 3-tert-butoxycarbonylmethoxy-6-chloro-benzo[b]thiophene-2-carboxylic acid methyl ester 7.70 (99%) as a slightly pink solid.

¹H NMR (400 MHz, CHLOROFORM-D) δ ppm 1.46 (s, 9H) 3.91 (s, 3H) 4.92 (s, 2H) 7.38 (dd, J=8.59, 1.77 Hz, 1H) 7.71 (d, J=2.02 Hz, 1H) 8.03 (d, J=8.59 Hz, 1H).

The third step of Scheme 15: 3-tert-Butoxycarbonylmethoxy-6-chloro-benzo[b]thiophene-2-carboxylic acid methyl ester (62 mg, 0.17 mmol) was hydrolyzed according to the procedure in the fifth step of Scheme 15 of Example 24 to yield 3-carboxymethoxy-6-chloro-benzo[b]thiophene-2-carboxylic acid, 7 mg (14%), as a white solid.

¹H NMR (400 MHz, DMSO-D6) δ ppm 4.99 (s, 2H) 7.51 (dd, J=8.59, 2.02 Hz, 1H) 7.97 (dd, J=8.59, 0.51 Hz, 1H) 8.14 (dd, J=1.77, 0.51 Hz, 1H).

### EXAMPLE 30

### 6-Bromo-3-carboxymethoxy-benzo[b]thiophene-2-carboxylic acid

The first step of Scheme 15: Lithium hydroxide (2.0 g, 48 mmol, 2 equiv) was added to a 0° C. 4-bromo-2-fluoro-benzoic acid methyl ester (5.5 g, 24 mmol, 1 equiv) and methyl thioglycolate (2.1 mL, 1 equiv) in DMF (30 mL). The mixture was stirred cold for 30 min, warmed to RT, added to water, and acidified with 1N HCl. The precipitate was filtered and dried under vacuum overnight yielding 4.89 g (72%) of 6-bromo-3-hydroxy-benzo[b]thiophene-2-carboxylic acid methyl ester.

¹H NMR (400 MHz, DMSO-D6) δ ppm 3.81 (s, 3H) 7.55 (dd, J=8.59, 1.26 Hz, 1H) 7.82 (d, J=8.34 Hz, 1H) 8.19 (s, 1H).

The second step of Scheme 15: Ethyl bromoacetate (3.0 mL, 27 mmol, 1.5 equiv) was added to a mixture of 6-bromo-3-hydroxy-benzo[b]thiophene-2-carboxylic acid methyl ester (5.1 g, 18 mmol) and NaOtBu (1.9 g, 1.1 equiv) in DMF (75 mL) and warmed to 80° C. When the reaction was complete by LC, water was added yielding a precipitate which was filtered a washed with water. Recrystallization in MeOH gave 6-bromo-3-ethoxycarbonylmethoxy-benzo[b]thiophene-2-carboxylic acid methyl ester 4.5 g (67%) as a slightly pink solid.

¹H NMR (400 MHz, CHLOROFORM-D) δ ppm 1.27 (t, J=7.07 Hz, 3H) 3.91 (s, 3H) 4.24 (q, J=7.24 Hz, 2H) 5.02 (s, 2H) 7.53 (dd, J=8.72, 1.64 Hz, 1H) 7.89 (dd, J=1.77, 0.51 Hz, 1H) 7.96 (dd, J=8.59, 0.51 Hz, 1H).

The third step of Scheme 15: 6-Bromo-3-ethoxycarbonylmethoxy-benzo[b]thiophene-2-carboxylic acid methyl ester (100 mg, 0.27 mmol) was hydrolyzed according to the procedure in the fifth step of Scheme 13 of Example 24 to yield 6-bromo-3-carboxymethoxy-benzo[b]thiophene-2-carboxylic acid, 73 mg (82%), as a white solid.

¹H NMR (400 MHz, DMSO-D6) δ ppm 4.99 (s, 2H) 7.64 (dd, J=8.59, 1.77 Hz, 1H) 7.90 (dd, J=8.59, 0.51 Hz, 1H) 8.29 (ds, J=1.77, 0.51 Hz, 1H).

### EXAMPLE 31

### 6-Chloro-3-(1-methoxycarbonyl-ethoxy)-benzo[b]thiophene-2-carboxylic acid methyl ester

The first step of Scheme 16: 6-Chloro-3-hydroxy-benzo[b]thiophene-2-carboxylic acid methyl ester (200 mg, 0.824 mmol) was dissolved in 10 mL DMF, followed by addition of K₂CO₃ (342 mg, 2.47 mmol) and 2-bromo-propionic acid methyl ester (110 μL, 0.99 mmol). The mixture was stirred at 70° C. for 16 hours. DMF was evaporated under reduced pressure, followed by addition of 15 mL of CH₂Cl₂. The organic layer was washed with water three times, brine once and then dried over anhydrous Na₂SO₄. The crude product was purified by silica chromatography eluting with a gradient of ethylacetate in hexane. Pure fractions were combined and evaporation of the solvent gave 6-chloro-3-(1-methoxycarbonyl-ethoxy)-benzo[b]thiophene-2-carboxylic acid methyl ester as a white solid (254 mg, 94%).

¹H NMR (400 MHz, CHLOROFORM-D) δ ppm 1.71 (d, J=6.82 Hz, 3H) 3.70 (s, 3H) 3.90 (s, 3H) 5.30 (q, J=6.82 Hz, 1H) 7.37 (dd, J=8.59, 1.77 Hz, 1H) 7.70 (d, J=1.52 Hz, 1H) 7.98 (d, J=8.59 Hz, 1H).

ESI-MS: m/e=351.15 [M+Na]⁺.

### EXAMPLE 32

### 3-(1-Carboxy-ethoxy)-6-chloro-benzo[b]thiophene-2-carboxylic acid

The second step of Scheme 16: 6-Chloro-3-(1-methoxycarbonyl-ethoxy)-benzo[b]thiophene-2-carboxylic acid methyl ester (250 mg, 0.76 mmol) was hydrolyzed according to the procedure in the third step of Scheme 1 of Example 1 to give 3-(1-carboxy-ethoxy)-6-chloro-benzo[b]thiophene-2-carboxylic acid as a white solid (211 mg, 92%).

¹H NMR (400 MHz, DMSO-D6) δ ppm 1.56 (d, J=6.82 Hz, 3H) 5.26 (d, J=6.82 Hz, 1H) 7.50 (dd, J=8.59, 2.02 Hz, 1H) 7.95 (d, J=8.59 Hz, 1H) 8.13 (d, J=1.77 Hz, 1H).

### EXAMPLE 33

### 6-Chloro-3-(ethoxycarbonyl-fluoro-methoxy)-benzo[b]thiophene-2-carboxylic acid methyl ester

The first step of Scheme 16: 6-Chloro-3-hydroxy-benzo[b]thiophene-2-carboxylic acid methyl ester (200 mg, 0.824 mmol) was dissolved in 10 mL DMF, followed by addition of K₂CO₃ (342 mg, 2.47 mmol) and bromo-fluoro-acetic acid ethyl ester (117 μL, 0.99 mmol). The mixture was stirred at 70° C. for 16 hours. DMF was evaporated under reduced pressure, followed by addition of 15 mL of CH₂Cl₂. The organic layer was washed with water three times, brine once and then dried over anhydrous Na₂SO₄. The crude product was purified by silica chromatography eluting with a gradient of ethylacetate in hexane. Pure fractions were combined and evaporation of the solvent gave 6-chloro-3-(ethoxycarbonyl-fluoro-methoxy)-benzo[b]thiophene-2-carboxylic acid methyl ester as a white solid (161 mg, 56%).

¹H NMR (400 MHz, CHLOROFORM-D) δ ppm 1.43 (t, J=7.07 Hz, 3H) 3.93 (s, 3H) 4.44 (q, J=7.07 Hz, 2H) 6.17 (d, J=59.37 Hz, 1H) 7.42 (dd, J=8.84, 1.77 Hz, 1H) 7.76 (d, J=1.77 Hz, 1H) 7.91 (d, J=8.59 Hz, 1H).

ESI-MS: m/e=369.15 [M+Na]⁺.

### EXAMPLE 34

### 3-(Carboxy-fluoro-methoxy)-6-chloro-benzo[b]thiophene-2-carboxylic acid

The second step of Scheme 16: 6-Chloro-3-(ethoxycarbonyl-fluoro-methoxy)-benzo[b]thiophene-2-carboxylic acid methyl ester (81 mg, 0.23 mmol) was hydrolyzed according to the procedure in the third step of Scheme 1 of Example 1 to give 3-(carboxy-fluoro-methoxy)-6-chloro-benzo[b]thiophene-2-carboxylic acid as a pink solid (>95%).

¹H NMR (400 MHz, DMSO-D6) δ ppm 6.23 (d, J=57.35 Hz, 1H) 7.55 (d, J=8.34 Hz, 2H) 7.81 (d, J=8.34 Hz, 2H) 8.20 (s, 1H).

### EXAMPLE 35

### (2-Carbamoyl-6-chloro-benzo[b]thiophen-3-yloxy)-acetic acid tert-butyl ester

The first step of Scheme 17: 3-tert-Butoxycarbonylmethoxy-6-chloro-benzo[b]thiophene-2-carboxylic acid methyl ester (175 mg, 049 mmol) was dissolved in 2 mL MeOH/1 mL THF in a pressure tube. The solution was cooled in dry ice/acetone bath and NH₃ gas was bubbled in until the total volume reached ˜6 mL. The tube was sealed and the mixture was stirred at room temperature for 16 hours. A white precipitate was formed and collected by filtration. The filtrate was then purified by silica chromatography eluting with a gradient of EtOAc and hexane. Pure fractions were combined and evaporation of the solvent gave (2-carbamoyl-6-chloro-benzo[b]thiophen-3-yloxy)-acetic acid tert-butyl ester as a white solid (77 mg, 46%).

¹H NMR (400 MHz, CHLOROFORM-D) δ ppm 1.52 (s, 9H) 4.84 (s, 2H) 5.84 (s, 1H) 7.37 (dd, J=8.72, 1.89 Hz, 1H) 7.71 (d, J=8.84 Hz, 1H) 7.80 (d, J=1.77 Hz, 1H) 8.47 (s, 1H).

### EXAMPLE 36

### 3-Carbamoylmethoxy-6-chloro-benzo[b]thiophene-2-carboxylic acid amide

The first step of Scheme 17: 3-Carbamoylmethoxy-6-chloro-benzo[b]thiophene-2-carboxylic acid amide was obtained as a white precipitate from the reaction described in the first step of Scheme 17 of Example 35 (70 mg, 50%).

¹H NMR (400 MHz, DMSO-D6) δ ppm 4.86 (s, 2H) 7.52 (dd, J=8.72, 1.89 Hz, 2H) 7.76 (s, 1H) 7.83 (m, 1H) 7.95 (d, J=8.59 Hz, 1H) 8.18 (d, J=1.77 Hz, 1H) 8.57 (s, 1H).

ESI-MS: m/e=307.65 [M+Na]⁺.

### EXAMPLE 37

### (2-Carbamoyl-6-chloro-benzo[b]thiophen-3-yloxy)-acetic acid

The second step of Scheme 17: (2-Carbamoyl-6-chloro-benzo[b]thiophen-3-yloxy)-acetic acid tert-butyl ester (60 mg, 0.18 mmol) was hydrolyzed according to the procedure in the first step of Scheme 2 of Example 2 to yield (2-carbamoyl-6-chloro-benzo[b]thiophen-3-yloxy)-acetic acid as a white solid (25 mg, 49%).

¹H NMR (400 MHz, DMSO-D6) δ ppm 5.10 (m, 2H) 7.49 (dd, J=8.84, 2.02 Hz, 1H) 7.89 (s, 1H) 8.04 (d, J=8.84 Hz, 1H) 8.17 (d, J=1.77 Hz, 1H) 8.33 (s, 1H).

### EXAMPLE 38

### 3-Carboxymethoxy-5-chloro-benzo[b]thiophene-2,6-dicarboxylic acid 6-benzyl ester

The first step of Scheme 18: To a solution of 2,5-dichloro-terephthalic acid (2.34 g, 5.7 mmol) and benzyl bromide (1.7 mL, 14.1 mmol) in N,N-dimethylformamide (10 mL) was added potassium carbonate (2.0 g, 14.7 mmol). The reaction was stirred for 4 hours and then diluted with ethyl acetate (300 mL). The organic layer was washed with sodium bicarbonate solution (100 mL) and dried over magnesium sulfate. The solution was filtered and the solvent was removed under reduced pressure to give 1.76 g (75%) of 2,5-dichloro-terephthalic acid dibenzyl ester as a white crystalline solid.

¹H NMR (400 MHz, CHLOROFORM-D) δ ppm 5.38 (m, 4H) 7.39 (m, 10H) 7.90 (s, 2H).

The second step of Scheme 18: A solution of 2,5-dichloro-terephthalic acid dibenzyl ester (1.12 g, 2.7 mmol), methylthioglycolate (242 μL, 2.7 mmol) and potassium carbonate (934 mg, 6.8 mmol) in N,N-dimethylformamide (14 mL) was heated to 100° C. for 4-6 hours. The reaction was diluted with ethyl acetate (300 mL) and washed with ammonium chloride (100 mL). The organic layer was dried over magnesium sulfate and filtered. The material was dissolved in N,N-dimethylformamide (15 mL) and treated with potassium carbonate (749 mg, 5.4 mmol) and ethylbromoacetate (601 μL, 5.4 mmol). After 30 minutes, another equivalent of ethyl bromoacetate (300 μL, 2.71 mmol) was added and the reaction was stirred for 2 hours. The reaction was diluted with ethyl acetate (300 mL) and washed with 3:1 water-saturated sodium chloride (100 mL). The aqueous layer was extracted with ethyl acetate (50 mL). The organic layers were combined and dried over magnesium sulfate. The solids were filtered and the solvent was removed under reduced pressure. The material was purified by CombiFlash column chromatography eluting with 0-50% ethyl acetate-hexane to give 640 mg (51%) of 5-chloro-3-ethoxycarbonylmethoxy-benzo[b]thiophene-2,6-dicarboxylic acid 6-benzyl ester 2-methyl ester as a white solid.

¹H NMR (400 MHz, CHLOROFORM-D) δ ppm 1.28 (t, J=7.20 Hz, 3H) 3.92 (s, 3H) 4.24 (q, J=7.16 Hz, 2H) 5.03 (s, 2H) 5.41 (s, 2H) 7.43 (m, 7H) 8.17 (s, 1H) 8.21 (s, 1 H).

The third step of Scheme 18: The procedure in the fifth step of Scheme 11 of Example 17 was followed to give a mixture of 3-carboxymethoxy-5-chloro-benzo[b]thiophene-2,6-dicarboxylic acid 6-benzyl ester and 3-carboxymethoxy-5-chloro-benzo[b]thiophene-2,6-dicarboxylic acid. The mixture was separated by preparatory reverse phase HPLC to give 10 mg of 3-carboxymethoxy-5-chloro-benzo[β]thiophene-2,6-dicarboxylic acid 6-benzyl ester as a white solid and 10 mg of 3-carboxymethoxy-5-chloro-benzo[β]thiophene-2,6-dicarboxylic acid as a white solid.

¹H NMR (400 MHz, DMSO-D6) δ ppm 5.05 (s, 2H) 5.43 (s, 2H) 7.32-7.61 (m, 5H) 8.15 (s, 1H) 8.27 (s, 1H).

### EXAMPLE 39

### 3-Carboxymethoxy-5-chloro-benzo[b]thiophene-2,6-dicarboxylic acid

The third step of Scheme 18: 3-Carboxymethoxy-5-chloro-benzo[b]thiophene-2,6-dicarboxylic acid was obtained as a white solid as described in Example 38.

¹H NMR (400 MHz, DMSO-D6) δ ppm 4.96 (d, J=7.07 Hz, 2H) 8.10 (m, 1H) 8.45 (s, 1H).

### EXAMPLE 40

### 3-Carboxymethoxy-6-phenylbenzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (90 mg, 0.25 mmol, 1 equiv), Pd₂(dba)₃ (4.4 mg), HP(t-Bu)₃BF₄ (2.9 mg), KF (43 mg, 3 equiv), and phenyl boronic acid (34 mg, 1.1 equiv) were used instead. The reaction vessel was heated to 60° C. for 5 hr. The crude mixture was absorbed onto silica and column chromatography, yielding 20 mg (20%) of impure 3-tert-butoxycarbonylmethoxy-6-phenylbenzo[b]thiophene-2-carboxylic acid methyl ester as a white solid. The material was advanced to the next step.

The second step of Scheme 19: Impure 3-tert-butoxycarbonylmethoxy-6-phenylbenzo[b]thiophene-2-carboxylic acid methyl ester (20 mg, 0.05 mmol) was hydrolyzed according to the procedure in the fifth step of Scheme 13 of Example 24. Work-up yielded 3-carboxymethoxy-6-phenylbenzo[b]thiophene-2-carboxylic acid 6 mg (38%) as a white solid.

¹H NMR (400 MHz, DMSO-D6) δ ppm 5.00 (s, 2H) 7.42 (tt, J=7.33, 1.20 Hz, 1H) 7.51 (t, J=7.58 Hz, 2H) 7.79 (m, 3H) 8.05 (d, J=8.34 Hz, 1H) 8.26 (d, J=0.76 Hz, 1H).

### EXAMPLE 41

### 6-Benzyl-3-carboxymethoxy-benzo[b]thiophene-2-carboxylic acid

The first step of Scheme 18: Following the procedure in the first step of Scheme 14 of Example 26, 3-tert-butoxycarbonylmethoxy-6-chloro-benzo[b]thiophene-2-carboxylic acid methyl ester (90 mg, 0.25 mmol, 1 equiv), Pd(OAc)₂ (2.2 mg), 2-(dicyclohexylphosphino)biphenyl (7 mg), and KF (33 mg) were used. The vessel was purged followed by the addition of THF and benzyl-9BBN [0.5M in THF](0.6 mL, 0.3 mmol, 1.2 equiv). After stirring for 12 hr at 60° C., the reaction mixture was absorbed onto celite and purified by column chromatography, yielding 94 mg (91%) of 6-benzyl-3-tert-butoxycarbonylmethoxy-benzo[b]thiophene-2-carboxylic acid methyl ester as a clear film.

¹H NMR (400 MHz, CHLOROFORM-D) δ ppm 1.45 (s, 9H) 3.87 (s, 3H) 4.07 (s, 2H) 4.89 (s, 2H) 7.31-7.15 (m, 6H) 7.48 (dd, J=1.52, 0.76 Hz, 1H) 7.99 (d, J=8.34 Hz, 1H).

The second step of Scheme 19: 6-Benzyl-3-tert-butoxycarbonylmethoxy-benzo[b]thiophene-2-carboxylic acid methyl ester (85 mg, 0.21 mmol) was hydrolyzed according to the procedure in the fifth step of Scheme 13 of Example 24. Work-up provided 31 mg (43%) of 6-benzyl-3-carboxymethoxy-benzo[b]thiophene-2-carboxylic acid as a white solid.

¹H NMR (400 MHz, DMSO-D6) δ ppm 4.06 (s, 2H) 4.71 (s, 2H) 7.22-7.16 (m, 1H) 7.33-7.25 (m, 5H), 7.76 (d, J=0.76 Hz, 1H) 7.80 (d, J=8.34 Hz, 1H).

### EXAMPLE 42

### 3-Carboxymethoxy-6-thiophen-3-yl-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (90 mg, 0.25 mmol, 1 equiv), Pd₂(dba)₃ (8.8 mg), HP(t-Bu)₃BF₄ (6 mg), KF (43 mg, 3 equiv), and 3-thiopheneboronic acid (35 mg, 1.1 equiv) were used at 60° C. for 21 hr. Following the work-up procedure in Example 26 yielded 3-tert-butoxycarbonylmethoxy-6-thiophen-3-yl-benzo[b]thiophene-2-carboxylic acid methyl ester 84 mg (83%).

¹H NMR (400 MHz, CHLOROFORM-D) δ ppm 1.47 (s, 9H) 3.90 (s, 3H) 4.93 (s, 2H) 6.68 (ddd, J=8.08, 2.27, 0.76 Hz, 1H) 6.92 (t, J=1.89 Hz, 1H) 7.00 (ddd, J=7.64, 1.58, 0.88 Hz, 1H) 7.27-7.19 (m, 2H) 7.59 (dd, J=8.34, 1.52 Hz, 1H) 7.84 (d, J=0.76 Hz, 1H) 8.09 (d, J=8.34 Hz, 1H).

The second step of Scheme 19: 3-tert-Butoxycarbonylmethoxy-6-thiophen-3-yl-benzo[b]thiophene-2-carboxylic acid methyl ester (40 mg, 0.1 mmol) was hydrolyzed according to the procedure in the fifth step of Scheme 13 of Example 24. Work-up according to Example 24 yielded 3-carboxymethoxy-6-thiophen-3-yl-benzo[b]thiophene-2-carboxylic acid, 18 mg (55%), as a white solid.

¹H NMR (400 MHz, DMSO-D6) δ ppm 5.00 (s, 2H) 7.70 (d, J=2.27 Hz, 2H) 7.87 (dd, J=8.34, 1.52 Hz, 1H) 8.00 (d, J=8.34 Hz, 1H) 8.06 (t, J=2.15 Hz, 1H) 8.32 (d, J=1.01 Hz, 1H).

### EXAMPLE 43

### 3-Carboxymethoxy-6-thiophen-2-yl-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (90 mg, 0.25 mmol, 1 equiv), 2-thiophene boronic acid (65 mg, 0.5 mmol, 1.5 equiv), Pd[P(t-Bu)₃]₂ (20 mg), and KF (50 mg) were used and the reaction was stirred at 60° C. for 48 hr. Work-up and column chromatography yielded 3-tert-butoxycarbonylmethoxy-6-thiophen-2-yl-benzo[b]thiophene-2-carboxylic acid methyl ester 86 mg (62%) as an off-white solid.

¹H NMR (400 MHz, CHLOROFORM-D) δ ppm 1.47 (s, 9H) 3.92 (s, 3H) 4.93 (s, 2H) 7.12 (dd, J=5.05, 3.54 Hz, 1H) 7.35 (dd, J=5.18, 1.14 Hz, 1H) 7.42 (dd, J=3.66, 1.14 Hz, 1H) 7.68 (dd, J=8.34, 1.52 Hz, 1H) 7.94 (dd, J=1.64, 0.63 Hz, 1H) 8.09 (dd, J=8.59, 0.76 Hz, 1H).

The second step of Scheme 19: 3-tert-Butoxycarbonylmethoxy-6-thiophen-2-yl-benzo[b]thiophene-2-carboxylic acid methyl ester (71 mg, 0.18 mmol) was hydrolyzed according to the procedure in the fifth step of Scheme 13 of Example 24 at 40° C. for 2 hr, then at room temperature for 12 hr. Work-up yielded 3-carboxymethoxy-6-thiophen-2-yl-benzo[b]thiophene-2-carboxylic acid, 4 mg (7%), as a yellow solid.

¹H NMR (400 MHz, DMSO-D6) δ ppm 5.00 (s, 2H) 7.19 (dd, J=5.05, 3.54 Hz, 1H) 7.64 (dd, J=5.05, 1.01 Hz, 1H) 7.68 (dd, J=3.66, 1.14 Hz, 1H) 7.78 (dd, J=8.46, 1.64 Hz, 1H) 7.99 (d, J=8.34 Hz, 1H) 8.27 (d, J=1.01 Hz, 1H).

### EXAMPLE 44

### 3-Carboxymethoxy-6-(4-hydroxyphenyl)-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (102 mg, 0.29 mmol, 1 equiv), 4-hydroxyphenylboronic acid (51 mg, 0.37 mmol, 1.3 equiv), Pd(OAc)₂ (8 mg), 2-(dicyclohexylphosphino)biphenyl (24 mg), and KF (50 mg) were used and the reaction mixture was stirred at 60° C. until the TLC showed the absence of the starting material. Work-up and column chromatography yielded 55 mg (47%) of 3-tert-butoxycarbonylmethoxy-6-(4-hydroxy-phenyl)-benzo[b]thiophene-2-carboxylic acid methyl ester as a white solid.

¹H NMR (400 MHz, CHLOROFORM-D) δ ppm 1.48 (s, 9H) 3.92 (s, 3H) 4.89 (s, 1H) 4.93 (s, 2H) 6.93 (d, J=8.84 Hz, 2H) 7.54 (d, J=8.84 Hz, 2H) 7.60 (dd, J=8.34, 1.52 Hz, 1H) 7.85 (d, J=1.01 Hz, 1H) 8.12 (d, J=9.10 Hz, 1H).

The second step of Scheme 19: 3-tert-Butoxycarbonylmethoxy-6-(4-hydroxy-phenyl)-benzo[b]thiophene-2-carboxylic acid methyl ester (50 mg, 0.12 mmol) was hydrolyzed according to the procedure in the fifth step of Scheme 13 of Example 24. Work-up yielded 3-carboxymethoxy-6-(4-hydroxyphenyl)-benzo[b]thiophene-2-carboxylic acid, 29 mg (69%), as a light-yellow solid.

¹H NMR (400 MHz, DMSO-D6) δ ppm 5.00 (s, 2H) 6.88 (d, J=8.84 Hz, 2H) 7.61 (d, J=8.59 Hz, 2H) 7.71 (dd, J=8.59, 1.52 Hz, 1H) 7.99 (d, J=8.34 Hz, 1H) 8.14 (s, 1H) 9.67 (s, 1H).

### EXAMPLE 45

### 3-Carboxymethoxy-6-(3-hydroxy-phenyl)-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (92 mg, 0.26 mmol, 1 equiv), 3-hydroxyphenylboronic acid (46 mg, 0.33 mmol, 1.3 equiv), Pd₂(dba)₃ (17 mg), HP(t-Bu)₃BF₄ (12 mg), and KF (50 mg) were stirred for 20 hr at 60° C. Following the work-up procedure in Example 26 yielded 70 mg (65%) of 3-tert-butoxycarbonylmethoxy-6-(3-hydroxy-phenyl)-benzo[b]thiophene-2-carboxylic acid methyl ester.

¹H NMR (400 MHz, CHLOROFORM-D) δ ppm 1.48 (s, 9H) 3.92 (s, 3H) 4.94 (s, 2H) 4.95 (s, 1H) 6.86 (ddd, J=7.96, 2.53, 0.88 Hz, 1H) 7.12 (dd, J=2.27, 1.77 Hz, 1H) 7.22 (ddd, J=7.71, 1.64, 1.01 Hz, 1H) 7.34 (t, J=7.83 Hz, 1H) 7.62 (dd, J=8.46, 1.64 Hz, 1H) 7.89 (d, J=1.01 Hz, 1H) 8.13 (d, J=8.59 Hz, 1H).

The second step of Scheme 19: 3-tert-Butoxycarbonylmethoxy-6-(3-hydroxy-phenyl)-benzo[b]thiophene-2-carboxylic acid methyl ester (54 mg, 0.13 mmol) was hydrolyzed according to the procedure in the fifth step of Scheme 13 of Example 24. Work-up according to Example 24 yielded 22 mg (49%) of 3-carboxymethoxy-6-(3-hydroxy-phenyl)-benzo[b]thiophene-2-carboxylic acid as a light-brown solid.

¹H NMR (400 MHz, DMSO-D6) δ ppm 5.01 (s, 2H) 6.82 (dd, J=7.71, 1.89 Hz, 1H) 7.11 (s, 1H) 7.18 (d, J=8.08 Hz, 1H) 7.30 (t, J=7.83 Hz, 1H) 7.71 (dd, J=8.59, 1.52 Hz, 1H) 8.03 (d, J=8.84 Hz, 1H) 8.19 (s, 1H) 9.60 (s, 1H).

### EXAMPLE 46

### 3-Carboxymethoxy-6-(4-nitro-phenyl)-benzo[b]thiophene-2-carboxylic acid

The first step of Scheme 19: Following the procedure in the first step of Scheme 14 of Example 26, 6-bromo-3-ethoxycarbonyl-methoxy-benzo[b]thiophene-2-carboxylic acid methyl ester (370 mg, 0.99 mmol, 1 equiv), 3-nitrophenylboronic acid (213 mg, 1.28 mmol, 1.3 equiv), Pd₂(dba)₃ (38 mg), HP(t-Bu)₃BF₄ (26 mg), and KF (148 mg) were stirred for 22 hr at room temperature, then 2 hr at 60° C. Following the work-up procedure in Example 26 yielded 349 mg (85%) of 3-ethoxycarbonyl-methoxy-6-(4-nitrophenyl)-benzo[b]thiophene-2-carboxylic acid methyl ester as a yellow solid.

¹H NMR (400 MHz, CHLOROFORM-D) δ ppm 1.29 (t, J=7.20 Hz, 3H) 3.94 (s, 3H) 4.26 (q, J=7.16 Hz, 2H) 5.07 (s, 2H) 7.67 (dd, J=8.46, 1.64 Hz, 1H) 7.81 (d, J=8.84 Hz, 2H) 7.97 (dd, J=1.52, 0.76 Hz, 1H) 8.22 (dd, J=8.46, 0.63 Hz, 1H) 8.34 (d, J=9.09 Hz, 2H).

HRMS (ESI+, m/z) calcd for [M+H]¹⁺, 416.07985, found, 416.07969.

The second step of Scheme 19: 3-Ethoxycarbonyl-methoxy-6-(4-nitrophenyl)-benzo[b]thiophene-2-carboxylic acid methyl ester (73 mg, 0.18 mmol) was hydrolyzed according to the procedure in the fifth step of Scheme 13 of Example 24. Work-up according to Example 24 yielded 55 mg (83%) of 3-carboxymethoxy-6-(4-nitro-phenyl)-benzo[b]thiophene-2-carboxylic acid as a golden solid.

¹H NMR (400 MHz, DMSO-D6) δ ppm 5.02 (s, 2H) 7.90 (dd, J=8.46, 1.64 Hz, 1H) 8.09 (m, 3H) 8.35 (d, J=9.10 Hz, 2H) 8.44 (d, J=1.01 Hz, 1H). HRMS (ESI−, m/z) calcd for [M−H]¹⁻, 372.01834, found, 372.01745.

### EXAMPLE 47

### 6-(4-Aminophenyl)-3-carboxymethoxy-benzo[b]thiophene-2-carboxylic acid

3-Ethoxycarbonyl-methoxy-6-(4-nitrophenyl)-benzo[b]thiophene-2-carboxylic acid methyl ester (259 mg, 0.62 mmol) was stirred under H₂ (1 atm) in a mixture of 10% Pd/C (40 mg), MeOH (20 mL), CHCl₃ (5 mL), and one drop of AcOH for 22 hr. The crude mixture was filtered through Celite and rotary evaporated. Recrystallization in EtOAc/MeOH yielded 176 mg (73%) of 6-(4-aminophenyl)-3-ethoxycarbonylmethoxy-benzo[b]thiophene-2-carboxylic acid methyl ester as a light-yellow solid.

¹H NMR (400 MHz, DMSO-D6) δ ppm 1.20 (t, J=7.20 Hz, 3H) 3.86 (s, 3H) 4.16 (q, J=7.16 Hz, 2H) 5.08 (s, 2H) 7.09 (d, J=7.83 Hz, 2H) 7.71 (d, J=8.34 Hz, 2H) 7.77 (dd, J=8.59, 1.77 Hz, 1H) 8.04 (d, J=8.59 Hz, 1H) 8.23 (d, J=1.26 Hz, 1H).

HRMS (ESI+, m/z) calcd for [M+H]¹⁺, 386.10567, found, 386.10535.

The second step of Scheme 19: 6-(4-Aminophenyl)-3-ethoxycarbonylmethoxy-benzo[b]thiophene-2-carboxylic acid methyl ester (72 mg, 0.19 mmol) was hydrolyzed according to the procedure in the fifth step of Scheme 13 of Example 24 at 40° C. for 16 hr. 52 mg (81%) of 6-(4-aminophenyl)-3-carboxymethoxy-benzo[b]thiophene-2-carboxylic acid was obtained as a golden solid following the procedure of the third step in Example 24.

¹H NMR (400 MHz, DMSO-D6) δ ppm 4.98 (s, 2H) 6.67 (d, J=8.84 Hz, 2H) 7.49 (d, J=8.84 Hz, 2H) 7.68 (dd, J=8.72, 1.64 Hz, 1H) 7.94 (d, J=8.59 Hz, 1H) 8.08 (d, J=1.01 Hz, 1H).

HRMS (ESI+, m/z) calcd for [M+H]¹⁺, 344.05872, found, 344.05885.

### EXAMPLE 48

### 6-(3-Amino-phenyl)-3-carboxymethoxy-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (90 mg, 0.25 mmol, 1 equiv), 3-aminophenylboronic acid (42 mg, 0.27 mmol, 1.1 equiv), Pd₂(dba)₃ (9 mg), HP(t-Bu)₃BF₄ (6 mg), and KF (43 mg) were stirred at 60° C. for 24 hr. Work-up and purification by column chromatography afforded 40 mg (39%) of 6-(3-aminophenyl)-3-tert-butoxycarbonylmethoxy-benzo[b]thiophene-2-carboxylic acid methyl ester as an off-white solid.

¹H NMR (400 MHz, CHLOROFORM-D) δ ppm 1.47 (s, 9H) 3.91 (s, 3H) 4.93 (s, 2H) 7.45-7.39 (m, 2H) 7.54 (dd, J=2.78, 1.52 Hz, 1H) 7.65 (dd, J=8.59, 1.52 Hz, 1H) 7.89 (dd, J=1.52, 0.76 Hz, 1H) 8.10 (dd, J=8.46, 0.63 Hz, 1H).

The second step of Scheme 19: 6-(3-Aminophenyl)-3-tert-butoxycarbonylmethoxy-benzo[b]thiophene-2-carboxylic acid methyl ester (84 mg, 0.2 mmol) was hydrolyzed according to the procedure in the fifth step of Scheme 13 of Example 24 to yield 40 mg of 6-(3-amino-phenyl)-3-carboxymethoxy-benzo[b]thiophene-2-carboxylic acid.

H NMR (400 MHz, DMSO-D6) δ ppm 5.01 (s, 2H) 6.63 (dd, J=7.96, 1.64 Hz, 1H) 6.90 (d, J=7.58 Hz, 1H) 6.95 (d, J=1.77 Hz, 1H) 7.15 (t, J=7.71 Hz, 1H) 7.67 (dd, J=8.46, 1.64 Hz, 1H) 8.02 (d, J=8.59 Hz, 1H) 8.11 (d, J=1.01 Hz, 1H).

### EXAMPLE 49

### 3-Carboxymethoxy-6-(4-dimethylaminophenyl)-benzo[b]thiophene-2-carboxylic acid

The first step of Scheme 19: Following the procedure in the first step of Scheme 14 of Example 26, 6-bromo-3-ethoxycarbonylmethoxy-benzo[b]thiophene-2-carboxylic acid methyl ester (66 mg, 0.18 mmol), 4-dimethylaminobenzene boronic acid (31 mg, 0.19 mmol), Pd₂(dba)₃ (13 mg), [P(t-Bu)₃]₂Pd (14 mg), and KF (22 mg) were stirred at room temperature for 4 days. Work-up and column chromatography yielded 38 mg (51%) of 6-(4-dimethylaminophenyl)-3-ethoxycarbonylmethoxy-benzo[b]thiophene-2-carboxylic acid methyl ester as a yellow-orange solid.

¹H NMR (400 MHz, CHLOROFORM-D) δ ppm 1.29 (t, J=7.07 Hz, 3H) 3.02 (s, 6H) 3.92 (s, 3H) 4.26 (q, J=7.16 Hz, 2H) 5.02 (s, 2H) 6.82 (d, J=9.10 Hz, 2H) 7.57 (d, J=8.84 Hz, 2H) 7.64 (dd, J=8.59, 1.52 Hz, 1H) 7.87 (dd, J=1.64, 0.63 Hz, 1H) 8.08 (dd, J=8.46, 0.63 Hz, 1H).

The second step of Scheme 19: 6-(4-Dimethylaminophenyl)-3-ethoxycarbonylmethoxy-benzo[b]thiophene-2-carboxylic acid methyl ester (37 mg, 0.09 mmol) was hydrolyzed according to the procedure in the fifth step of Scheme 13 of Example 24. Work-up afforded 29 mg (88%) of 3-carboxymethoxy-6-(4-dimethylaminophenyl)-benzo[b]thiophene-2-carboxylic acid as a yellow-gold solid.

¹H NMR (400 MHz, DMSO-D6) δ ppm 2.97 (s, 6H) 4.98 (s, 2H) 6.83 (d, J=9.10 Hz, 2H) 7.65 (d, J=8.84 Hz, 2H) 7.73 (dd, J=8.59, 1.77 Hz, 1H) 7.97 (d, J=8.34 Hz, 1H) 8.15 (d, J=1.01 Hz, 1H).

### EXAMPLE 50

### 3-Carboxymethoxy-6-(4-cyanophenyl)-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (109 mg, 0.31 mmol, 1 equiv), 4-cyanophenyl boronic acid (68 mg, 0.46 mmol, 1.5equiv), [P(t-Bu)₃]₂Pd (20 mg), and KF (54 mg) were used and the mixture was stirred at 60° C. for 48 hr. Work-up and column chromatography yielded 99 mg (76%) of 3-tert-butoxycarbonylmethoxy-6-(4-cyanophenyl)-benzo[b]thiophene-2-carboxylic acid methyl ester.

¹H NMR (400 MHz, CHLOROFORM-D) δ ppm 1.48 (s, 9H) 3.94 (s, 3H) 4.95 (s, 2H) 7.63 (dd, J=8.46, 1.64 Hz, 1H) 7.76 (s, 4H) 7.93 (dd, J=1.52, 0.51 Hz, 1H) 8.21 (dd, J=8.59, 0.51 Hz, 1H).

The second step of Scheme 19: 3-tert-Butoxycarbonylmethoxy-6-(4-cyanophenyl)-benzo[b]thiophene-2-carboxylic acid methyl ester (99 mg, 0.23 mmol) was hydrolyzed according to the procedure in the fifth step of Scheme 13 of Example 24 for 17 hr at room temperature, then warmed to 40° C. for 1 hr. Work-up yielded 69 mg (83%) of 3-carboxymethoxy-6-(4-cyanophenyl)-benzo[b]thiophene-2-carboxylic acid as an orange solid.

¹H NMR (400 MHz, DMSO-D6) δ ppm 4.97 (s, 2H) 7.86 (dd, J=8.46, 1.64 Hz, 1H) 8.03-7.95 (m, 4H) 8.07 (d, J=8.08 Hz, 1H) 8.39 (d, J=1.01 Hz, 1H).

### EXAMPLE 51

### 3-Carboxymethoxy-6-(2-methoxyphenyl)-benzo[b]thiophene-2-carboxylic acid

The first step of Scheme 19: Following the procedure in the first step of Scheme 14 of Example 26, 6-bromo-3-ethoxycarbonylmethoxy-benzo[b]thiophene-2-carboxylic acid methyl ester (85 mg, 0.23 mmol), 2-methoxyphenyl boronic acid (58 mg, 0.38 mmol), Pd₂(dba)₃ (17 mg), HP(t-Bu)₃BF₄ (13 mg), and KF (28 mg) were stirred at room temperature for 17 hr. Work-up and purification by column chromatography afforded 77 mg (84%) of 3-ethoxycarbonylmethoxy-6-(2-methoxyphenyl)-benzo[b]thiophene-2-carboxylic acid methyl ester as a white film.

¹H NMR (400 MHz, CHLOROFORM-D) δ ppm 1.30 (t, J=7.07 Hz, 3H) 3.83 (s, 3H) 3.92 (s, 3H) 4.27 (q, J=7.07 Hz, 2H) 5.01 (s, 2H) 7.02 (dd, J=8.72, 0.88 Hz, 1H) 7.06 (td, J=7.52, 1.14 Hz, 1H) 7.39-7.34 (m, 2H) 7.60 (dd, J=8.59, 1.52 Hz, 1H) 7.88 (dd, J=1.52, 0.76 Hz, 1H) 8.10 (dd, J=8.34, 0.76 Hz, 1H).

The second step of Scheme 19: 3-Ethoxycarbonylmethoxy-6-(2-methoxyphenyl)-benzo[b]thiophene-2-carboxylic acid methyl ester (77 mg, 0.19 mmol) was hydrolyzed according to the procedure in the fifth step of Scheme 13 of Example 24. Work-up provided 53 mg (78%) of 3-carboxymethoxy-6-(2-methoxyphenyl)-benzo[b]thiophene-2-carboxylic acid as a white solid.

¹H NMR (400 MHz, DMSO-D6) δ ppm 3.79 (s, 3H) 5.01 (s, 2H) 7.07 (td, J=7.52, 1.14 Hz, 1H) 7.16 (dd, J=8.34, 0.76 Hz, 1H) 7.44-7.34 (m, 2H) 7.57 (dd, J=8.34, 1.52 Hz, 1H) 8.03-7.96 (m, J=9.47, 0.88 Hz, 2H).

### EXAMPLE 52

### 3-Carboxymethoxy-6-phenylamino-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (88 mg, 0.25 mmol, 1 equiv), 4-(4,4,5,5-tetramethyl-1,3,2-dioxaborolan-2-yl)analine (70 mg, 0.31 mmol, 1.3 equiv), Pd(OAc)₂ (8 mg), 2-(dicyclohexylphosphino)biphenyl (24 mg), and KF (50 mg) were used and the mixture was stirred at 60° C. for 3 days. Work-up and column chromatography yielded 46 mg (45%) of the unexpected product, 3-tert-butoxycarbonylmethoxy-6-phenylamino-benzo[b]thiophene-2-carboxylic acid methyl ester.

¹H NMR (400 MHz, CHLOROFORM-D) δ ppm 1.47 (s, 9H) 3.88 (s, 3H) 4.90 (s, 2H) 5.95 (s, 1H) 7.08-7.02 (m, 3H) 7.17 (d, J=8.59 Hz, 1H) 7.37-7.31 (m, 3H) 7.94 (d, J=8.34 Hz, 1H).

The second step of Scheme 19: 3-tert-Butoxycarbonylmethoxy-6-phenylamino-benzo[b]thiophene-2-carboxylic acid methyl ester (30 mg, 0.07 mmol) was hydrolyzed according to the procedure in the fifth step of Scheme 13 of Example 24. Work-up yielded 11 mg of 3-carboxymethoxy-6-phenylamino-benzo[b]thiophene-2-carboxylic acid as a yellow solid.

¹H NMR (400 MHz, DMSO-D6) δ ppm 4.94 (s, 2H) 6.94 (t, J=7.33 Hz, 1H) 7.13 (dd, J=8.84, 2.02 Hz, 1H) 7.19 (d, J=7.58 Hz, 2H) 7.31 (m, 2H) 7.46 (d, J=2.02 Hz, 1H) 7.79 (d, J=8.84 Hz, 1H) 8.65 (s, 1H).

### EXAMPLE 53

### 3-Carboxymethoxy-6-naphthalen-1-yl-benzo[b]thiophene-2-carboxylic acid

The first step of Scheme 19: Following the procedure in the first step of Scheme 14 of Example 26, 3-tert-butoxycarbonylmethoxy-6-chloro-benzo[b]thiophene-2-carboxylic acid methyl ester (93 mg, 0.26 mmol, 1 equiv), Pd₂(dba)₃ (17 mg), HP(t-Bu)₃BF₄ (12 mg), and KF (50 mg) were stirred at 60° C. for 24 hr. Work-up and column chromatography provided 97 mg of 3-tert-butoxycarbonylmethoxy-6-naphthalen-1-yl-benzo[b]thiophene-2-carboxylic acid methyl ester as a yellow film.

¹H NMR (400 MHz, CHLOROFORM-D) δ ppm 1.50 (s, 9H) 3.94 (s, 3H) 4.98 (s, 2H) 7.60-7.34 (m, 6H) 7.84 (dd, J=1.39, 0.63 Hz, 1H) 7.95-7.85 (m, 2H) 8.20 (dd, J=8.21, 0.63 Hz, 1H).

The second step of Scheme 19: 3-tert-Butoxycarbonylmethoxy-6-naphthalen-1-yl-benzo[b]thiophene-2-carboxylic acid methyl ester (74 mg, 0.17 mmol) was hydrolyzed according to the procedure in the fifth step of Scheme 13 of Example 24. Work-up yielded 30 mg (48%) of 3-carboxymethoxy-6-naphthalen-1-yl-benzo[b]thiophene-2-carboxylic acid as a white solid.

¹H NMR (400 MHz, DMSO-D6) δ ppm 4.92 (s, 2H) 7.65-7.48 (m, 5H) 7.81 (d, J=8.34 Hz, 1H) 8.09-7.98 (m, 4H).

### EXAMPLE 54

### 3-Carboxymethoxy-6-(4-methoxy-phenyl)-benzo[b]thiophene-2-carboxylic acid dilithio salt

The first step of Scheme 19: Following the procedure in the first step of Scheme 14 of Example 26, 3-tert-butoxycarbonylmethoxy-6-chloro-benzo[b]thiophene-2-carboxylic acid methyl ester (109 mg, 0.31 mmol, 1 equiv), 4-methoxyphenyl boronic acid (60 mg, 0.39 mmol), Pd₂(dba)₃ (17 mg), HP(t-Bu)₃BF₄ (12 mg), and KF (46 mg) were stirred at 60° C. for 3 days. Work-up and column chromatography yielded 66 mg (50%) of 3-tert-butoxycarbonylmethoxy-6-(4-methoxy-phenyl)-benzo[b]thiophene-2-carboxylic acid methyl ester as a white solid.

¹H NMR (400 MHz, CHLOROFORM-D) δ ppm 1.48 (s, 9H) 3.87 (s, 3H) 3.92 (s, 3 H) 4.93 (s, 2H) 7.01 (d, J=8.84 Hz, 2H) 7.64-7.58 (m, 3H) 8.12 (dd, J=8.46, 0.63 Hz, 1H).

The second step of Scheme 19: 3-tert-Butoxycarbonylmethoxy-6-(4-methoxy-phenyl)-benzo[b]thiophene-2-carboxylic acid methyl ester (66 mg, 0.15 mmol) was hydrolyzed according to the procedure in the fifth step of Scheme 13 of Example 24. Without acidifying the mixture, filtration of the white solid yielded 25 mg of 3-carboxymethoxy-6-(4-methoxy-phenyl)-benzo[b]thiophene-2-carboxylic acid dilithio salt.

¹H NMR (400 MHz, DMSO-D6) δ ppm 3.81 (s, 3H) 4.41 (s, 2H) 7.04 (d, J=8.84 Hz, 2H) 7.62 (d, J=7.58 Hz, 1H) 7.71 (d, J=8.84 Hz, 2H) 7.90 (d, J=8.34 Hz, 1H) 8.06 (s, 1H).

### EXAMPLE 55

### 3-Carboxymethoxy-6-(3-formyl-thiophen-2-yl)-benzo[b]thiophene-2-carboxylic acid

The first step of Scheme 19: Following the procedure in the first step of Scheme 14 of Example 26, 6-bromo-3-ethoxycarbonylmethoxy-benzo[b]thiophene-2-carboxylic acid methyl ester (305 mg, 0.82 mmol), 3-formyl-2-thiophene boronic acid (153 mg, 0.98 mmol), Pd₂(dba)₃ (38 mg), [P(t-Bu)₃]₂Pd (26 mg), and KF (95 mg) were stirred at room temperature for 24 hr. Work-up and column chromatography afforded 268 mg (81%) of 3-ethoxycarbonylmethoxy-6-(3-formyl-thiophen-2-yl)-benzo[b]thiophene-2-carboxylic acid methyl ester as a yellow-white solid.

¹H NMR (400 MHz, CDCl₃) δ ppm 1.29 (t, J=7.07 Hz, 3H) 3.94 (s, 3H) 4.26 (q, J=7.07 Hz, 2H) 5.07 (s, 2H) 7.34 (dd, J=5.43, 0.88 Hz, 1H) 7.56 (dd, J=8.34, 1.52 Hz, 1H) 7.61 (d, J=5.31 Hz, 1H) 7.86 (d, J=0.76 Hz, 1H) 8.21 (d, J=8.84 Hz, 1H) 9.92 (s, 1H).

The second step of Scheme 19: 3-Ethoxycarbonylmethoxy-6-(3-formyl-thiophen-2-yl)-benzo[b]thiophene-2-carboxylic acid methyl ester (61 mg, 0.15 mmol) was hydrolyzed according to the procedure in the fifth step of Scheme 13 of Example 24 at 50° C. until LC indicated disappearance of starting material. Work-up yielded 44 mg (81%) of 3-carboxymethoxy-6-(3-formyl-thiophen-2-yl)-benzo[b]thiophene-2-carboxylic acid as an off-white solid.

¹H NMR (400 MHz, DMSO-D6) δ ppm 5.03 (s, 2H) 7.55 (d, J=5.31 Hz, 1H) 7.71 (dd, J=8.46, 1.64 Hz, 1H) 7.75 (dd, J=5.31, 0.76 Hz, 1H) 8.10 (dd, J=8.34, 0.50 Hz, 1H) 8.30 (dd, J=1.64, 0.63 Hz, 1H) 9.85 (s, 1H).

### EXAMPLE 56

### 3-Carboxymethoxy-6-(3-hydroxymethyl-thiophen-2-yl)-benzo[b]thiophene-2-carboxylic acid

Sodium borohydride (10 mg, 0.26 mmol) was added to a chilled (0° C.) solution of 3-ethoxycarbonylmethoxy-6-(3-formyl-thiophen-2-yl)-benzo[b]thiophene-2-carboxylic acid methyl ester (91 mg, 0.22 mmol) in THF (3 mL). After 5 min, the mixture was warmed to room temperature and stirred for 1.5 hr. Water (5 mL) was added to the reaction followed by extraction with EtOAc. The organic layers were combined and concentrated. Column chromatography yielded 41 mg (45%) of 3-ethoxycarbonylmethoxy-6-(3-hydroxymethyl-thiophen-2-yl)-benzo[b]thiophene-2-carboxylic acid methyl ester.

¹H NMR (400 MHz, CHLOROFORM-D) δ ppm 1.29 (t, J=7.07 Hz, 4H) 1.74-1.69 (m, 1H) 3.93 (s, 3H) 4.26 (q, J=7.07 Hz, 2H) 4.72 (d, J=4.80 Hz, 2H) 5.04 (s, 2H) 7.22 (d, J=5.31 Hz, 1H) 7.35 (d, J=5.05 Hz, 1H) 7.57 (dd, J=8.46, 1.64 Hz, 1H) 7.88 (dd, J=1.39, 0.63 Hz, 1H) 8.13 (dd, J=8.46, 0.63 Hz, 1H).

The second step of Scheme 19: 3-Ethoxycarbonylmethoxy-6-(3-hydroxymethyl-thiophen-2-yl)-benzo[b]thiophene-2-carboxylic acid methyl ester (41 mg, 0.10 mmol) was hydrolyzed according to the procedure in the fifth step of Scheme 13 of Example 24. Work-up afforded 33 mg (89%) of 3-carboxymethoxy-6-(3-hydroxymethyl-thiophen-2-yl)-benzo[b]thiophene-2-carboxylic acid as an off-white solid.

¹H NMR (400 MHz, DMSO-D6) δ ppm 4.51 (s, 2H) 5.01 (s, 2H) 5.30 (s, 1H) 7.22 (d, J=5.05 Hz, 1H) 7.58 (d, J=5.05 Hz, 1H) 7.61 (dd, J=8.59, 1.52 Hz, 1H) 8.03 (d, J=8.34 Hz, 1H) 8.08 (d, J=1.01 Hz, 1H).

HRMS (ESI−, m/z) calcd for [M−H]⁻, 363.00025, found, 362.99953.

### EXAMPLE 57

### 6-Chloro-3-(3-cyanopropoxy)benzo[b]thiophene-2-carboxylic acid

The first step of Scheme 20: A solution of 6-chloro-3-hydroxybenzo[b]thiophene-2-carboxylic acid methyl ester (100 mg, 0.4 mmol), potassium carbonate (113 mg, 1.2 eq), 4-chlorobutyronitrile (106 μL, 2.5 eq), and potassium iodide (66 mg, 1 eq) in DMF (2 mL) were heated at 60° C. for 2 h. The cooled solution was diluted with ethyl acetate (100 mL) and washed with water (3×50 mL) and brine, dried over anhydrous magnesium sulfate, filtered and concentrated in vacuo. Flash chromatography (silica, 10-30% ethyl acetate/hexanes) provided 6-chloro-3-(3-cyanopropoxy)-benzo[b]thiophene-2-carboxylic acid methyl ester (113 mg, 89%) as a clear, colorless oil which solidified upon standing.

¹H NMR (400 MHz, chloroform-D) δ ppm 2.23 (m, 2H) 2.76 (t, J=7.20 Hz, 2H) 3.92 (s, 3H) 4.42 (t, J=5.68 Hz, 2H) 7.38 (dd, J=8.59, 1.77 Hz, 1H) 7.76 (m, 2H).

The second step of Scheme 20: A solution of 6-chloro-3-(3-cyanopropoxy)benzo[b]thiophene-2-carboxylic acid methyl ester (113 mg, 0.37 mmol) and lithium hydroxide (45 mg, 3 eq) in tetrahydrofuran (4 mL), methanol (1 mL), and water (1 mL) was stirred at room temperature overnight. The reaction was neutralized with aq. ammonium chloride, and the volatiles were removed in vacuo. The aqueous solution was acidified with aq. hydrochloric acid, the precipitate was collected by vacuum filtration, and dried under high vacuum to provide 6-chloro-3-(3-cyanopropoxy)benzo[b]thiophene-2-carboxylic acid (99 mg, 92%) as a white solid.

¹H NMR (400 MHz, DMSO-D6) δ ppm 2.03-2.15 (m, 2H) 2.76 (t, J=7.20 Hz, 2H) 3.32 (bs, 1H) 4.35 (t, J=5.94 Hz, 2H) 7.50 (dd, J=8.72, 1.89 Hz, 1H) 7.92 (d, J=8.59 Hz, 1H) 8.17 (d, J=1.77 Hz, 1H).

### EXAMPLE 58

### 6-Benzyloxy-3-carboxymethoxy-benzo[b]thiophene-2-carboxylic acid

The first step of Scheme 21: A solution of 4-benzyloxy-2-hydroxybenzoic acid methyl ester (2.0 g, 7.8 mmol), N,N-dimethylthiocarbamoyl chloride (1.9 g, 2eq), DABCO (1.74 g, 2eq) in DMF (10 mL) were heated at 50° C. overnight. The cooled solution was diluted with ethyl acetate (100 mL), washed with water (3×50 mL), brine, dried over anhydrous magnesium sulfate, filtered and concentrated in vacuo. Recrystallization from methanol provided 4-benzyloxy-2-dimethylthiocarbamoyloxybenzoic acid methyl ester (2.43 g, 90%) as a yellow crystalline solid.

¹H NMR (400 MHz, CHLOROFORM-D) δ ppm 3.39 (s, 3H) 3.47 (s, 3H) 3.81 (s, 3H) 6.73 (d, J=2.53 Hz, 1H) 6.90 (dd, J=8.84, 2.53 Hz, 1H) 7.38 (m, 5H) 7.98 (d, J=8.84 Hz, 1H).

The second step of Scheme 21: A solution of 4-benzyloxy-2-dimethylthiocarbamoyloxybenzoic acid methyl ester (2.43 g, 7 mmol) in diphenyl ether (13 mL) was heated at 220° C. overnight. The cooled homogeneous solution was poured into 25 mL of hexanes and stirred at 10° C. for 20 min. The resulting tan solid was filtered and dried under high vacuum to provide crude 4-benzyloxy-2-dimethylcarbamoylsulfanylbenzoic acid methyl ester (1.84 g, 76%).

The third step of Scheme 21: A solution of 4-benzyloxy-2-dimethylcarbamoylsulfanylbenzoic acid methyl ester (1.83 g, 5.3 mmol) and potassium hydroxide (0.88 g, 2.5 eq) in tetrahydrofuran (10 mL), methanol (5 mL), and water (3 mL) was heated at reflux overnight. The cooled solution was diluted with ethyl acetate (200 mL) and water (30 mL), then acidified with aq. hydrochloric acid. The organic phase was washed with water (2×50 mL) and brine, dried over anhydrous magnesium sulfate, filtered, and concentrated in vacuo to provide crude 4-benzyloxy-2-mercaptobenzoic acid. This intermediate was dissolved in a mixture of concentrated sulfuric acid (10 drops) and methanol (150 mL) and heated at reflux overnight. The cooled solution was neutralized with aqueous sodium bicarbonate, diluted with ethyl acetate (300 mL) and washed with water (3×50 mL). The organic phase was dried over anhydrous magnesium sulfate, filtered, and concentrated in vacuo. Flash chromatography (20% ethyl acetate:hexanes) provided crude 4-benzyloxy-2-mercaptobenzoic acid methyl ester (488 mg, 34%, 2 steps).

¹H NMR (400 MHz, CHLOROFORM-D) δ ppm 3.88 (s, 3H) 5.06 & 5.08 (ps, rotamers, 2H) 6.89 (d, J=2.27 Hz, 1H) 7.38 (m, 5H) 7.98 (d, J=9.10 Hz, 1H).

The fourth step of Scheme 21: To a solution of 4-benzyloxy-2-mercaptobenzoic acid methyl ester (488 mg, 1.8 mmol) and tert-butyl bromoacetate (0.523 mL, 2eq) in dimethylformamide (4 mL) was added sodium methoxide (0.24 g, 2.5 eq). After stirring for 1 h at room temperature, a second portion of sodium methoxide (0.24 g, 2.5 eq) was added to the reaction. After stirring at room temperature for an additional 2 h, the reaction was acidified with aqueous hydrochloric acid, diluted with ethyl acetate (100 mL), washed with water (3×30 mL), aqueous sodium acetate (20 mL), and brine. The resulting organic phase was dried over anhydrous magnesium sulfate, filtered, and concentrated in vacuo. Flash chromatography (10% ethyl acetate/hexanes) provided 6-benzyloxy-3-hydroxy-benzo[b]thiophene-2-carboxylic acid t-butyl ester (491 mg, 77%).

¹H NMR (400 MHz, CHLOROFORM-D) δ ppm 1.61 (s, 9H) 5.13 (s, 2H) 7.07 (dd, J=8.84, 2.27 Hz, 1H) 7.21 (d, J=2.02 Hz, 1H) 7.34-7.48 (m, 5H) 7.80 (d, J=9.10 Hz, 1H).

The fifth step of Scheme 21: 6-Benzyloxy-3-hydroxy-benzo[b]thiophene-2-carboxylic acid t-butyl ester (491 mg, 1.4 mmol) was converted to 6-benzyloxy-3-tert-butoxycarbonyl-methoxybenzo[b]thiophene-2-carboxylic acid t-butyl ester (650 mg, 99%), following the procedure in the first step of Scheme 20 of Example 57.

¹H NMR (400 MHz, CHLOROFORM-D) δ ppm 1.46 (s, 9H) 1.59 (s, 9H) 4.86 (s, 2H) 5.13 (s, 2H) 7.08 (dd, J=8.97, 2.15 Hz, 1H) 7.19 (d, J=2.02 Hz, 1H) 7.33-7.47 (m, 5H) 7.95 (d, J=8.84 Hz, 1H).

The sixth step of Scheme 21: 6-Benzyloxy-3-tert-butoxycarbonylmethoxy-benzo[b]thiophene-2-carboxylic acid t-butyl ester (50 mg, 1 mmol) was converted to 6-benzyloxy-3-carboxymethoxy-benzo[b]thiophene-2-carboxylic acid (40 mg, 100%), following the procedure in the second step of Scheme 20 of Example 57.

¹H NMR (400 MHz, DMSO-D6) δ ppm 4.95 (s, 2H) 5.20 (s, 2H) 7.15 (dd, J=8.97, 2.15 Hz, 1H) 7.33-7.37 (m, J=7.20, 7.20 Hz, 1H) 7.38-7.45 (m, J=7.33, 7.33 Hz, 2H) 7.46-7.52 (m, 2H) 13.12 (s, 1H).

### EXAMPLE 59

### 6-Acetyl-3-carboxymethoxy-benzo[b]thiophene-2-carboxylic acid

The first step of Scheme 22: To a solution of 3-tert-butoxycarbonylmethoxy-6-chlorobenzo[b]thiophene-2-carboxylic acid methyl ester (2.0 g, 5.6 mmol) in 1-methyl-2-pyrrolidinone (7 mL) was added tris(dibenzylideneacetone)dipalladium (258 mg, 5 mol %), tri-t-butylphosphine tetrafluoroborate (326 mg, 4 eq), tributyl(1-ethoxyvinyl)tin (2.3 mL, 1.2 eq), and cesium fluoride (1.87 g, 2.2 eq). The resulting suspension was stirred at room temperature for 48 h. The reaction solution was diluted with ethyl acetate (200 mL) filtered, and washed with water (4×30 mL) and brine, dried over anhydrous magnesium sulfate, filtered, and concentrated in vacuo. The crude vinyl ether was dissolved in methylene chloride (10 mL) and to this solution was added boron trifluoride etherate (0.96 mL, 1.5 eq), tetrabutylammonium fluoride hydrate (1.6 g, 1.0 eq) and water (182 μL). After stirring at room temperature for 200 min, the reaction was neutralized with aqueous sodium bicarbonate, diluted with ethyl acetate (100 mL), washed with water (2×30 mL) and brine, dried over anhydrous magnesium sulfate, filtered, and concentrated in vacuo. Flash chromatography (10% ethyl acetate/hexanes) provided 6-acetyl-3-tert-butoxycarbonylmethoxybenzo[b]thiophene-2-carboxylic acid methyl ester (1.26 g, 61%, 2 steps).

¹H NMR (400 MHz, CHLOROFORM-D) δ ppm 1.46 (s, 9H) 2.69 (s, 3H) 3.94 (s, 3H) 4.94 (s, 2H) 8.17 (d, J=8.59 Hz, 1H) 8.34 (s, 1H).

The second step of Scheme 22: 6-Acetyl-3-tert-butoxycarbonylmethoxy-benzo[b]thiophene-2-carboxylic acid methyl ester (50 mg, 0.14 mmol) was converted to 6-acetyl-3-carboxymethoxy-benzo[b]thiophene-2-carboxylic acid, following the procedure in the second step of Scheme 20 of Example 57.

¹H NMR (400 MHz, DMSO-D6) δ ppm 2.67 (s, 3H) 5.02 (s, 2H) 7.98 (dd, J=8.59, 1.52 Hz, 1H) 8.08 (dd, J=8.59, 0.76 Hz, 1H) 8.65 (d, J=0.76 Hz, 1H).

### EXAMPLE 60

### 3-Carboxymethoxy-6-(1-hydroxyiminoethyl)benzo[b]thiophene-2-carboxylic acid

The first step of Scheme 32: A mixture of 6-acetyl-3-tert-butoxycarbonylmethoxybenzo[b]thiophene-2-carboxylic acid methyl ester (100 mg, 0.28 mmol), hydroxylamine hydrochloride (40 mg, 2 eq), and pyridine (67 μL, 3eq) in methanol (10 mL) was heated at reflux for 2 h. The cooled solution was acidified with aqueous hydrogen chloride, diluted with ethyl acetate (100 mL), washed with water (2×30 mL) and brine, dried over anhydrous magnesium sulfate, filtered, and concentrated in vacuo. Flash chromatography (20% ethyl acetate/hexanes) provided 3-tert-butoxycarbonylmethoxy-6-(1-hydroxyiminoethyl)benzo[b]thiophene-2-carboxylic acid methyl ester (98 mg, 92%) as a white solid.

¹H NMR (400 MHz, CHLOROFORM-D) δ ppm 1.47 (s, 9H) 2.35 (s, 3H) 3.92 (s, 3H) 4.93 (s, 2H) 7.73 (dd, J=8.59, 1.52 Hz, 1H) 7.94 (d, J=0.76 Hz, 1H) 8.08 (d, J=8.59 Hz, 1H) 8.68 (s, 1H).

The second step of Scheme 23: 3-tert-Butoxycarbonylmethoxy-6-(1-hydroxy-iminoethyl)benzo[b]thiophene-2-carboxylic acid methyl ester (98 mg, 0.26 mmol) was converted to 3-carboxymethoxy-6-(1-hydroxyiminoethyl)benzo[b]thiophene-2-carboxylic acid (69 mg, 90 mg), following the procedure in the second step of Scheme 20 of Example 57.

¹H NMR (400 MHz, DMSO-D6) δ ppm 2.23 (s, 3H) 5.00 (s, 2H) 7.83 (dd, J=8.59, 1.26 Hz, 1H) 7.95 (d, J=8.59 Hz, 1H) 8.18 (d, J=0.76 Hz, 1H) 11.43 (s, 1H).

### EXAMPLE 61

### 3-Carboxymethoxy-6-(3-fluorophenyl)benzo-[b]thiophene-2-carboxylic acid

The first step of Scheme 19: A solution of 3-tert-butoxycarbonylmethoxy-6-chlorobenzo[b]thiophene-2-carboxylic acid methyl ester (100 mg, 0.28 mmol), tris(dibenzylideneacetone)dipalladium (26 mg, 10 mol %), tri-t-butylphosphine fluoroborate (32 mg, 4 eq), potassium fluoride (49 mg, 2 eq), 3-fluorophenylboronic acid (59 mg, 1.5 eq) in tetrahydrofuran (800 μL) was stirred at 60° C. overnight. The crude reaction solution was absorbed onto silica gel, evaporated, and flash chromatographed (5% ethyl acetate/hexanes). The resulting diester was converted to 3-carboxymethoxy-6-(3-fluorophenyl)benzo-[b]thiophene-2-carboxylic acid (14 mg, 14%, 2 steps), following the procedure in the second step of Scheme 20 of Example 57.

¹H NMR (400 MHz, DMSO-D6) δ ppm 5.02 (s, 2H) 7.2-7.32 (m, 1H) 7.51-7.59 (m, 1H) 7.62-7.68 (m, 2H) 7.84 (dd, J=8.59, 1.52 Hz, 1H) 8.06 (d, J=8.59 Hz, 1H) 8.33 (d, J=1.01 Hz, 1H).

### EXAMPLE 62

### 3-Carboxymethoxy-6-(2-fluorophenyl)-benzo[b]thiophene-2-carboxylic acid

The first step of Scheme 19: 3-tert-Butoxycarbonylmethoxy-6-chlorobenzo[b]thiophene-2-carboxylic acid methyl ester (100 mg, 0.28 mmol) was converted to 3-tert-butoxycarbonylmethoxy-6-(2-fluorophenyl)benzo[b]thiophene-2-carboxylic acid methyl ester 35 mg, 30%), following the procedure in Example 61.

¹H NMR (400 MHz, CHLOROFORM-D) δ ppm 1.48 (s, 9H) 4.94 (s, 2H) 7.20 (m, 2H) 7.36 (m, 1H) 7.49 (m, 1H) 7.60 (m, 1H) 7.90 (m, 1H) 8.16 (dd, J=8.46, 0.63 Hz, 1H).

The second step of Scheme 19: 3-tert-Butoxycarbonylmethoxy-6-(2-fluorophenyl)benzo[b]thiophene-2-carboxylic acid methyl ester (35 mg, 0.08 mmol) was converted to 3-carboxymethoxy-6-(2-fluorophenyl)-benzo[b]thiophene-2-carboxylic acid (21 mg, 72%), following the procedure in Example 57.

¹H NMR (400 MHz, DMSO-D6) δ ppm 5.03 (s, 2H) 7.36 (m, 2H) 7.48 (m, 1H) 7.64 (m, 2H) 8.07 (d, J=8.34 Hz, 1H) 8.14 (s, 1H).

### EXAMPLE 63

### 3-Carboxymethoxy-benzo[b]thiophene-2,6-dicarboxylic acid 6-tert-butyl ester

The first step of Scheme 24: A solution of 2-nitroterephthalic acid 1-methyl ester (5.0 g, 22 mmol), isobutylene (20 mL), concentrated sulfinuric acid (1 mL) in dioxane (25 mL) was sealed in a Parr bottle and shaken at room temperature overnight. A needle was inserted through the silicone stopper to release excessive pressure, the vessel was carefully removed from the shaker apparatus, and the reaction solution was stirred open to the atmosphere for 1 h. The solution was diluted with ethyl acetate (300 mL) and water (50 mL), neutralized with aq. sodium hydroxide, washed with water (3×50 mL) and brine, dried over anhydrous magnesium sulfate, filtered and concentrated in vacuo to provide 2-nitroterephthalic acid 4-tert-butyl ester 1-methyl ester (3.38 g, 55%).

¹H NMR (400 MHz, CHLOROFORM-D) δ ppm 1.62 (s, 9H) 3.95 (s, 3H) 7.79 (d, J=7.83 Hz, 1H) 8.27 (dd, J=7.83, 1.52 Hz, 1H) 8.48 (d, J=1.26 Hz, 1H).

The second step of Scheme 24: A solution of 2-nitroterephthalic acid 4-tert-butyl ester 1-methyl ester (3.3 g, 12 mmol) and methyl thioglycolate (1.6 mL, 1.5 eq) in dimethylformamide (15 mL) was cooled to 0° C., and lithium hydroxide monohydrate (0.99 g, 2 eq) was added over a 15 minute period. The reaction was stirred at 0° C. for an additional 30 min, allowed to warm to room temperature, and stirred an additional 2 h. The solution was acidified, diluted with ethyl acetate (300 mL), and washed with water (3×50 mL) and brine, dried over anhydrous magnesium sulfate, filtered, and concentrated in vacuo to provide 3-hydroxybenzo[b]thiophene-2,6-dicarboxylic acid 6-tert-butyl ester 2-methyl ester (3.10 g, 84%) as a yellow solid.

¹H NMR (400 MHz, CHLOROFORM-D) δ ppm 1.63 (s, 9H) 3.97 (s, 3H) 7.96 (dm, J=8.4 Hz, 1H) 8.00 (dm, J=8.4 Hz, 1H) 8.39 (s, 1H).

The third step of Scheme 24: 3-Hydroxybenzo[b]thiophene-2,6-dicarboxylic acid 6-tert-butyl ester 2-methyl ester (3.1 g, 10 mmol) was converted to 3-methoxycarbonylmethoxy-benzo[b]thiophene-2,6-dicarboxylic acid 6-tert-butyl ester 2-methyl ester (3.17 g, 83%), following the procedure in the first step of Scheme 20 of Example 57.

¹H NMR (400 MHz, CHLOROFORM-D) δ ppm 1.61 (s, 9H) 3.77 (s, 3H) 3.91 (s, 3H) 5.02 (s, 2H) 7.99 (dd, J=8.80, 1.30 Hz, 1H) 8.08 (d, J=8.80 Hz, 1H) 8.37 (s, 1H).

The fourth step of Scheme 24: 3-Methoxycarbonylmethoxy-benzo[b]thiophene-2,6-dicarboxylic acid 6-tert-butyl ester 2-methyl ester was converted to 3-carboxymethoxy-benzo[b]thiophene-2,6-dicarboxylic acid 6-tert-butyl ester (80 mg, 87%), following the procedure in the second step of Scheme 20 of Example 57.

¹H NMR (400 MHz, DMSO-D6) δ ppm 1.58 (s, 9H) 4.80 (s, 2H) 7.90 (dd, J=8.59, 1.52 Hz, 1H) 7.98 (d, J=8.59 Hz, 1H) 8.48 (s, 1H).

### EXAMPLE 64

### 6-Carbamoyl-3-carboxymethoxy-benzo[b]thiophene-2-carboxylic acid

The first step of Scheme 25: A solution of 3-methoxycarbonylmethoxy-benzo[b]thiophene-2,6-dicarboxylic acid 6-tert-butyl ester 2-methyl ester (2.80 g, 7.4 mmol) and trifluoroacetic acid (6 mL) in dichloromethane (40 mL) were stirred at room temperature 4.5 h. The solution was concentrated in vacuo to provide 3-methoxycarbonylmethoxy-benzo[b]thiophene-2,6-dicarboxylic acid 2-methyl ester (2.60 g, 108%).

¹H NMR (400 MHz, DMSO-D6) δ ppm 3.69 (s, 3H) 3.87 (s, 3H) 5.10 (s, 2H) 8.00 (dd, J=8.30, 1.52 Hz, 1H) 8.09 (d, J=8.34 Hz, 1H) 8.62 (s, 1H).

The second step of Scheme 25: A solution of 3-methoxycarbonylmethoxybenzo-[b]thiophene-2,6-dicarboxylic acid 2-methyl ester (84 mg, 0.3 mmol) and 1,1′-carbonyldiimidazole (90 mg, 2 eq) in tetrahydrofuran (3 mL) was heated at reflux for 2.5 h. Ammonia was bubbled into the cooled solution for 5 min, and the reaction was then stirred for an additional 30 min. The solution was diluted with ethyl acetate (50 mL), acidified with aq. hydrochloric acid, washed with water (2×10 mL), brine, dried over magnesium sulfate, filtered, and concentrated in vacuo to provide 6-carbamoyl-3-methoxycarbonylmethoxy-benzo[b]thiophene-2-carboxylic acid methyl ester (79 mg, 87%).

¹H NMR (400 MHz, DMSO-D6) δ ppm 3.69 (s, 3H) 3.87 (s, 3H) 5.10 (s, 2H) 7.57 (bs, 1H) 7.95 (dd, J=8.46, 1.39 Hz, 1H) 8.05 (d, J=8.34 Hz, 4H) 8.15 (bs, 1H) 8.47 (s, 1H).

The third step of Scheme 25: 6-Carbamoyl-3-methoxycarbonylmethoxy-benzo[b]thiophene-2-carboxylic acid methyl ester (74 mg, 0.23 mmol) was converted to 6-carbamoyl-3-carboxymethoxy-benzo[b]thiophene-2-carboxylic acid (29 mg, 43%), following the procedure in the second step of Scheme 20 of Example 57.

¹H NMR (400 MHz, DMSO-D6) δ ppm 5.01 (s, 2H) 7.54 (bs, 1H) 7.93 (dd, J=8.46, 1.39 Hz, 1H) 8.03 (d, J=8.34 Hz, 1H) 8.14 (bs, 1H) 8.44 (s, 1H) 13.32 (bs, 2H).

### EXAMPLE 65

### 3-Carboxymethoxy-6-dimethylcarbamoyl-benzo[b]thiophene-2-carboxylic acid

The second step of Scheme 25: 3-Methoxycarbonylmethoxybenzo[b]thiophene-2,6-dicarboxylic acid 2-methyl ester (75 mg, 0.23 mmol) and dimethylamine (0.46 mL, 4 eq, THF soln.) were converted to 6-dimethylcarbamoyl-3-methoxycarbonylmethoxy-benzo[b]thiophene-2-carboxylic acid methyl ester (73 mg, 90%), following the procedure in Example 64.

¹H NMR (400 MHz, DMSO-D6) 8 ppm 2.93 (bs, 3H) 3.02 (bs, 3H) 3.70 (s, 3H) 3.86 (s, 3H) 5.09 (s, 2H) 7.50 (dd, J=8.34, 1.26 Hz, 1H) 8.05 (d, J=8.34 Hz, 1H) 8.06 (bs, 1H).

The third step of Scheme 25: 6-Dimethylcarbamoyl-3-methoxycarbonylmethoxy-benzo[b]thiophene-2-carboxylic acid methyl ester (68 mg, 0.2 mmol) was converted to 3-carboxymethoxy-6-dimethylcarbamoyl-benzo[b]thiophene-2-carboxylic acid (48 mg, 78%), following the procedure in the second step of Scheme 20 of Example 57.

¹H NMR (400 MHz, DMSO-D6) δ ppm 2.93 (bs, 3H) 3.02 (bs, 3H) 5.01 (s, 2H) 7.48 (dd, J=8.59, 1.26 Hz, 1H) 8.02 (m, 8H).

### EXAMPLE 66

### 6-Benzylcarbamoyl-3-carboxymethoxy-benzo[b]thiophene-2-carboxylic acid

The second step of Scheme 25: A solution of 3-methoxycarbonylmethoxy-benzo[b]thiophene-2,6-dicarboxylic acid 2-methyl ester (100 mg, 0.3 mmol), benzylamine (52 μL, 1.3 eq), 1-(3-dimethylaminopropyl)-3-ethylcarbodiimide hydrochloride (65 mg, 1.1 eq), and 4-(dimethylamino)pyridine (4 mg, 10 mol %) in dimethylformamide (2 mL) was stirred overnight. The reaction was diluted with ethyl acetate, washed with 10% aq. hydrogen chloride, water and brine, dried over magnesium sulfate, filtered and evaporated. Flash chromatography (40% ethyl acetate/hexane) provided 6-benzylcarbamoyl-3-methoxycarbonylmethoxybenzo[b]thiophene-2-carboxylic acid methyl ester (84 mg, 63%) as a clear, colorless oil.

¹H NMR (400 MHz, CHLOROFORM-D) δ ppm 3.77 (s, 3H) 3.91 (s, 3H) 4.65 (d, J=5.56 Hz, 2H) 5.02 (s, 2H) 6.74 (t, J=5.43 Hz, 1H) 7.31 (m, 5H) 7.75 (dd, J=8.59, 1.52 Hz, 1H) 8.06 (dd, J=8.46, 0.63 Hz, 1H) 8.19 (dd, J=1.52, 0.76 Hz, 1H).

The third step of Scheme 25: 6-Benzylcarbamoyl-3-methoxycarbonylmethoxy-benzo[b]thiophene-2-carboxylic acid methyl ester (68 mg, 0.17 mmol) was converted to 6-benzylcarbamoyl-3-carboxymethoxybenzo[b]thiophene-2-carboxylic acid (57 mg, 90%), following the procedure in the second step of Scheme 20 of Example 57.

¹H NMR (400 MHz, DMSO-D6) δ ppm 4.52 (d, J=6.06 Hz, 2H) 4.87 (s, 2H) 7.25 (m, 1H) 7.33 (m, 4H) 7.93 (dd, J=8.34, 1.52 Hz, 1H) 8.00 (d, J=8.34 Hz, 1H) 8.44 (m, 1H) 9.20 (t, J=6.06 Hz, 1H).

HRMS (ESI+) calcd for C₁₉H₁₅NO₆S 386.06929; found 386.06953.

### EXAMPLE 67

### 6-(Benzylmethylcarbamoyl)-3-carboxymethoxy-benzo[b]thiophene-2-carboxylic acid

The second step of Scheme 25: 3-Methoxycarbonylmethoxybenzo[b]thiophene-2,6-dicarboxylic acid 2-methyl ester (100 mg, 0.3 mmol) was converted to 6-(benzyl-methyl-carbamoyl)-3-methoxycarbonylmethoxy-benzo[b]thiophene-2-carboxylic acid methyl ester (84 mg, 63%) as a clear, colorless oil, following the procedure in Example 66.

¹H NMR (400 MHz, CHLOROFORM-D) δ ppm 2.89 & 3.07 (s, rotamers, 2H) 3.78 (s, 3H) 3.91 (s, 3H) 4.53 & 4.78 (s, rotamers, 3H) 7.17 (m, 1H) 7.34 (m, 4H) 7.49 (dd, J=8.34, 1.52 Hz, 1H) 7.84 (s, 1H) 8.10 (m, 1H).

The third step of Scheme 25: 6-(Benzylmethylcarbamoyl)-3-methoxycarbonylmethoxy-benzo[b]thiophene-2-carboxylic acid methyl ester (84 mg, 0.32 mmol) was converted to 6-(benzylmethylcarbamoyl)-3-carboxymethoxy-benzo[b]thiophene-2-carboxylic acid (54 mg, 68%), following the procedure in the second step of Scheme 20 of Example 57.

¹H NMR (400 MHz, DMSO-D6) δ ppm 2.89 (m, 3H) 4.49 & 4.72 (s, rotamers, 2H) 5.01 (s, 2H) 7.37 (m, 6H) 8.07 (m, 2H).

HRMS (ESI) calcd for C₂₀H₁₇NO₆S 400.08494; found 400.0847.

### EXAMPLE 68

### 3-Carboxmethoxy-6-(2-methyl-5-phenyl-2H-pyrazol-3-ylcarbamoyl)-benzo[b]thiophene-2-carboxylic acid

The second step of Scheme 25: A solution of 3-methoxycarbonylmethoxy-benzo[b]thiophene-2,6-dicarboxylic acid 2-methyl ester (16, 3 mmol) and thionyl chloride (10 mL) in toluene (50 mL) was heated at reflux overnight. The cooled solution was concentrated in vacuo, and the residual thionyl chloride was removed by azeotroping with added toluene. The crude acid chloride, 6-chlorocarbonyl-3-methoxycarbonylmethoxy-benzo[b]thiophene-2-carboxylic acid methyl ester was used without additional purification. A solution of 6-chlorocarbonyl-3-methoxycarbonyl-methoxybenzo[b]thiophene-2-carboxylic acid methyl ester (75 mg, 0.22 mmol), 5-amino-1-methyl-3-phenylpyrazole (57 mg, 1.5 eq), and pyridine (53 μL, 3 eq) in dichloromethane (3 mL) was stirred at room temperature for 4 h. The reaction was quenched with aq. sodium bicarbonate, diluted with ethyl acetate (50 mL) and washed with brine, dried over magnesium sulfate, filtered and concentrated in vacuo to provide a crude yellow sold which was recrystallized from ethanol to provide 3-methoxycarbonylmethoxy-6-(2-methyl-5-phenyl-2H-pyrazol-3-ylcarbamoyl)-benzo[b]thiophene-2-carboxylic acid methyl ester (83 mg, 79%) as a white solid.

¹H NMR (400 MHz, DMSO-D6) δ ppm 3.70 (s, 3H) 3.80 (s, 3H) 3.88 (s, 3H) 5.13 (s, 2H) 6.76 (s, 1H) 7.30 (m, 1H) 7.41 (m, 2H) 7.80 (m, 2H) 8.06 (dd, J=8.60, 1.52 Hz 1H) 8.16 (d, J=8.40 Hz, 1H) 8.65 (d, J=0.76 Hz, 1H) 10.63 (s, 1H).

The third step of Scheme 25: 3-Methoxycarbonylmethoxy-6-(2-methyl-5-phenyl-2H— pyrazol-3-ylcarbamoyl)-benzo[b]thiophene-2-carboxylic acid methyl ester 78 mg, 0.2 mmol) was converted to 3-carboxymethoxy-6-(2-methyl-5-phenyl-2H-pyrazol-3-ylcarbamoyl)-benzo[b]thiophene-2-carboxylic acid (67 mg, 93%), following the procedure in the second step of Scheme 20 of Example 57.

¹H NMR (400 MHz, DMSO-D6) δ ppm 3.80 (s, 3H) 4.96 (s, 2H) 6.76 (s, 1H) 7.30 (m, 1H) 7.41 (m, 2H) 7.80 (m, 2H) 8.02 (dd, J=8.30, 1.30 Hz, 1H) 8.10 (d, J=8.60 Hz, 1H) 8.60 (d, J=0.76 Hz, 1H) 10.61 (s, 1H).

### EXAMPLE 69

### 3-carboxymethoxy-6-(3-methyl-isothiazol-5-ylcarbamoyl)-benzo[b]thiophene-2-carboxylic acid

The second step of Scheme 25: 6-Chlorocarbonyl-3-methoxycarbonylmethoxy-benzo[b]thiophene-2-carboxylic acid methyl ester (75 mg, 0.22 mmol) was converted to 3-methoxycarbonylmethoxy-6-(3-methyl-isothiazol-5-ylcarbamoyl)-benzo[b]thiophene-2-carboxylic acid methyl ester (44 mg, 48%), following the procedure in Example 68.

¹H NMR (400 MHz, DMSO-D6) δ ppm 2.37 (s, 3H) 3.70 (s, 3H) 3.88 (s, 3H) 5.13 (s, 2H) 6.95 (s, 1H) 8.09 (dd, J=8.59, 1.26 Hz, 1H) 8.18 (d, J=8.34 Hz, 1H) 8.67 (d, J=0.76 Hz, 1H) 12.44 (s, 1H).

The third step of Scheme 25: 3-Methoxycarbonylmethoxy-6-(3-methyl-isothiazol-5-ylcarbamoyl)-benzo[b]thiophene-2-carboxylic acid methyl ester (39 mg, 0.1 mmol) was converted to 3-carboxymethoxy-6-(3-methyl-isothiazol-5-ylcarbamoyl)-benzo[b]thiophene-2-carboxylic acid (35 mg, 96%), following the procedure in the second step of Scheme 20 of Example 57.

¹H NMR (400 MHz, DMSO-D6) δ ppm 2.37 (s, 3H) 5.04 (s, 2H) 7.08 (s, 1H) 8.12-8.19 (m, 2H) 8.77 (s, 1H) 12.79 (s, 1H).

HRMS (ESI) calcd for C₁₆H₁₂N₂O₆S₂ 393.02096; found 393.02099.

### EXAMPLE 70

### 3-Carboxymethoxy-6-(thiazol-2-ylcarbamoyl)benzo[b]thiophene-2-carboxylic acid

The second step of Scheme 25: 6-Chlorocarbonyl-3-methoxycarbonylmethoxy-benzo[b]thiophene-2-carboxylic acid methyl ester (75 mg, 0.22 mmol) and 2-aminothiazole (33 mg, 1.5 eq) were converted to 3-methoxycarbonylmethoxy-6-(thiazol-2-ylcarbamoyl)-benzo[b]thiophene-2-carboxylic acid methyl ester (26 mg, 29%), following the procedure in Example 68.

¹H NMR (400 MHz, DMSO-D6) δ ppm 3.70 (s, 3H) 3.88 (s, 3H) 5.12 (s, 2H) 7.32 (d, J=3.54 Hz, 1H) 7.59 (d, J=3.54 Hz, 1H) 8.12-8.15 (m, 2H).

The third step of Scheme 25: 3-Methoxycarbonylmethoxy-6-(thiazol-2-ylcarbamoyl)-benzo[b]thiophene-2-carboxylic acid methyl ester (48 mg, 0.12 mmol) was converted to 3-carboxymethoxy-6-(thiazol-2-ylcarbamoyl)benzo[b]thiophene-2-carboxylic acid (42 mg, 93%), following the procedure in the second step of Scheme 20 of Example 57.

¹H NMR (400 MHz, DMSO-D6) δ ppm 5.04 (s, 2H) 7.32 (d, J=3.79 Hz, 1H) 7.59 (d, J=3.54 Hz, 1H) 8.08-8.14 (m, 2H) 8.70 (s, 1H). HRMS (ESI) calcd for C₁₅H₁₀N₂O₆S₂ 379.00531; found 379.00485.

### EXAMPLE 71

### 3-carboxymethoxy-6-(5-methyl-1-phenyl-1H-pyrazol-3-ylcarbamoyl)-benzo[b]thiophene-2-carboxylic acid

The second step of Scheme 25: 6-Chlorocarbonyl-3-methoxycarbonylmethoxy-benzo[b]thiophene-2-carboxylic acid methyl ester (75 mg, 0.22 mmol) and 3-amino-5-methyl-1-phenylpyrazole (57 mg, 1.5 eq) were converted to 3-methoxycarbonylmethoxy-6-(5-methyl-1-phenyl-1H-pyrazol-3-ylcarbamoyl)-benzo[b]thiophene-2-carboxylic acid methyl ester, following the procedure in Example 68.

¹H NMR (400 MHz, DMSO-D6) δ ppm 3.33 (s, 3H) 3.69 (s, 3H) 3.87 (s, 3H) 5.11 (s, 2H) 6.31 (s, 1H) 7.31 (m, 1H) 7.45 (m, 2H) 7.54 (m, 2H) 7.92 (d, J=8.59 Hz, 1H) 8.10 (d, J=8.34 Hz, 1H) 8.49 (s, 1H) 10.57 (s, 1H).

The third step of Scheme 25: 3-Methoxycarbonylmethoxy-6-(5-methyl-1-phenyl-1H— pyrazol-3-ylcarbamoyl)-benzo[b]thiophene-2-carboxylic acid methyl ester (53 mg, 0.11 mmol) was converted to 3-carboxymethoxy-6-(5-methyl-1-phenyl-1H-pyrazol-3-ylcarbamoyl)-benzo[b]thiophene-2-carboxylic acid (39 mg, 79%), following the procedure in the second step of Scheme 20 of Example 57.

¹H NMR (400 MHz, DMSO-D6) δ ppm 5.02 (s, 2H) 6.31 (s, 1H) 7.31 (m, 1H) 7.45 (m, 5H) 7.54 (m, 2H) 7.89 (m, 1H) 8.07 (d, J=8.59 Hz, 1H) 8.45 (s, 1H) 10.54 (s, 1H).

HRMS (ESI) calcd for C₂₂H₁₇N₃O₆S 452.09109; found 452.08967.

### EXAMPLE 72

### 3-Carboxymethoxy-6-(3-methylisoxazol-5-ylcarbamoyl)-benzo[b]thiophene-2-carboxylic acid

The second step of Scheme 25: 6-Chlorocarbonyl-3-methoxycarbonylmethoxy-benzo[b]thiophene-2-carboxylic acid methyl ester (75 mg, 0.22 mmol) and 5-amino-3-methylisoxazole (32 mg, 1.5 eq) was converted to 3-methoxycarbonylmethoxy-6-(3-methyl-isoxazol-5-ylcarbamoyl)-benzo[b]thiophene-2-carboxylic acid methyl ester (52 mg, 59%), following the procedure in Example 68.

¹H NMR (400 MHz, DMSO-D6) δ ppm 3.33 (s, 3H) 3.70 (s, 3H) 3.88 (s, 3H) 5.12 (s, 2H) 6.37 (s, 1H) 8.05 (m, 1H) 8.13 (m, 1H) 8.65 (s, 1H) 12.11 (s, 1H).

The third step of Scheme 25: 3-Methoxycarbonylmethoxy-6-(3-methylisoxazol-5-ylcarbamoyl)-benzo[b]thiophene-2-carboxylic acid methyl ester (47 mg, 0.12 mmol) was converted to 3-carboxymethoxy-6-(3-methylisoxazol-5-ylcarbamoyl)-benzo[b]thiophene-2-carboxylic acid (22 mg, 50%), following the procedure in the second step of Scheme 20 of Example 57.

¹H NMR (400 MHz, DMSO-D6) δ ppm 5.04 (s, 2H) 6.36 (s, 1H) 8.03 (dd, J=8.59, 1.52 Hz, 1H) 8.09-8.13 (m, 1H) 8.61 (s, 1H) 12.09 (s, 1H).

HRMS (ESI) calcd for C₁₆H₁₂N₂O₇S 377.0438; found 377.04352.

### EXAMPLE 73

### 6-tert-Butoxycarbonylamino-3-carboxymethoxy-benzo[b]thiophene-2-carboxylic acid

The first step of Scheme 26: A solution of 3-methoxycarbonylmethoxy-benzo[b]thiophene-2,6-dicarboxylic acid 2-methyl ester (1.0 g, 3.1 mmol), triethylamine (0.86 mL, 2 eq), tert-butyl alcohol (2 mL, 6.5 eq), and diphenylphosphoryl azide (0.93 mL, 1.4 eq) in toluene (20 mL) were heated at 100° C. for 18 h. The cooled solution was diluted with ethyl acetate, washed with aqueous sodium bicarbonate and brine, dried over magnesium sulfate, filtered and evaporated. Flash chromatography (30% ethyl acetate/hexanes) provided 6-tert-butoxycarbonylamino-3-methoxycarbonylmethoxy-benzo[b]thiophene-2-carboxylic acid methyl ester (815 mg, 67%) as a white solid.

¹H NMR (400 MHz, CHLOROFORM-D) δ ppm 1.54 (s, 9H) 3.79 (s, 3H) 3.89 (s, 3H) 5.02 (s, 2H) 6.68 (bs, 1H) 7.09 (dd, J=8.84, 2.02 Hz, 1H) 7.95 (dd, J=8.84, 0.51 Hz, 1H) 8.14 (bs, 1H).

The fourth step of Scheme 26: 6-tert-Butoxycarbonylamino-3-methoxycarbonyl-methoxybenzo[b]thiophene-2-carboxylic acid methyl ester (50 mg, 0.1 mmol) was converted to 6-tert-butoxycarbonylamino-3-carboxymethoxy-benzo[b]thiophene-2-carboxylic acid (35 mg, 75%), following the procedure in the second step of Scheme 20 of Example 57.

¹H NMR (400 MHz, DMSO-D6) δ ppm 1.50 (s, 9H) 4.91 (s, 2H) 7.42 (dd, J=8.84, 1.77 Hz, 1H) 7.82 (d, J=8.84 Hz, 1H) 8.10 (d, J=1.26 Hz, 1H) 9.73 (s, 1H).

HRMS (ESI) calcd for C₁₆H₁₇NO₇S 368.07985; found 368.07987.

### EXAMPLE 74

### 6-Benzoylamino-3-carboxymethoxybenzo[b]thiophene-2-carboxylic acid

The second step of Scheme 26: A solution of 6-tert-butoxycarbonylamino-3-methoxycarbonylmethoxy-benzo[b]thiophene-2-carboxylic acid methyl ester (800 mg, 2 mmol) and trifluoroacetic acid (2.5 mL) in dichloromethane (20 mL) was stirred at room temperature for 40 min. The solution was concentrated in vacuo to provide crude 6-amino-3-methoxycarbonylmethoxy-benzo[b]thiophene-2-carboxylic acid methyl ester (756 mg, 128%) as a pink solid.

¹H NMR (400 MHz, DMSO-D6) δ ppm 3.69 (s, 3H) 3.78 (s, 3H) 5.00 (s, 2H) 6.82 (dd, J=8.84, 2.02 Hz, 1H) 6.97 (d, J=1.77 Hz, 1H) 7.68 (d, J=8.59 Hz, 1H).

The third step of Scheme 26: A solution of 6-amino-3-methoxycarbonylmethoxy-benzo[b]thiophene-2-carboxylic acid methyl ester (80 mg, 0.3 mmol), benzoyl chloride (63 μL, 2 eq), 4-(dimethylamino)pyridine (33 mg, 1 eq), and triethylamine (189 μL, 5 eq) in dichloromethane (10 mL) was stirred at room temperature for 2 h. The reaction was quenched with methanol, absorbed on silica gel, and flash chromatographed (40% ethyl acetate/hexanes) to provide 6-benzoylamino-3-methoxycarbonylmethoxy-benzo[b]thiophene-2-carboxylic acid methyl ester (37 mg, 34%) as a white solid.

¹H NMR (400 MHz, CHLOROFORM-D) δ ppm 3.79 (s, 3H) 3.90 (s, 3H) 5.03 (s, 2H) 7.36 (dd, J=8.72, 1.89 Hz, 1H) 7.46-7.53 (m, 2H) 7.54-7.60 (m, 1H) 7.86-7.92 (m, 2H) 8.01 (d, J=9.10 Hz, 1H) 8.12 (s, 1H) 8.49 (d, J=1.77 Hz, 1H).

The fourth step of Scheme 26: 6-Benzoylamino-3-methoxycarbonylmethoxybenzo-[b]thiophene-2-carboxylic acid methyl ester (37 mg, 0.01 mmol) was converted to 6-benzoylamino-3-carboxymethoxybenzo[b]thiophene-2-carboxylic acid (28 mg, 81%), following the procedure in the second step of Scheme 20 of Example 57.

¹H NMR (400 MHz, DMSO-D6) δ ppm 4.82 (s, 2H) 7.52-7.65 (m, 3H) 7.75 (dd, J=8.84, 2.02 Hz, 1H) 7.90 (d, J=8.84 Hz, 1H) 7.96-8.03 (m, 2H) 8.48 (d, J=1.77 Hz, 1H) 10.53 (s, 1H).

HRMS (ESI) calcd for C₁₈H₁₃NO₆S 372.05364; found 372.05328.

### EXAMPLE 75

### 6-(3-Benzylureido)-3-carboxymethoxybenzo[b]thiophene-2-carboxylic acid

The third step of Scheme 26: A solution of 6-amino-3-methoxycarbonylmethoxy-benzo[b]thiophene-2-carboxylic acid methyl ester (75 mg, 0.25 mmol) and benzyl isocyanate (63 μL, 2 eq) in dichloroethane was stirred at room temperature for 45 min. The reaction was diluted with ethyl acetate and washed with water and brine, dried over magnesium sulfate, filtered, and concentrated in vacuo. Recrystallization from ethanol provided 6-(3-benzylureido)-3-methoxycarbonylmethoxy-benzo[b]thiophene-2-carboxylic acid methyl ester (28 mg, 26%) as a white solid.

¹H NMR (400 MHz, DMSO-D6) δ ppm 3.69 (s, 3H) 3.81 (s, 3H) 4.32 (d, J=6.06 Hz, 2H) 5.04 (s, 2H) 6.80 (t, J=5.94 Hz, 3H) 7.17-7.42 (m, 6H) 8.19 (d, J=1.52 Hz, 1H) 9.00 (s, 1H).

The fourth step of Scheme 26: 6-(3-Benzylureido)-3-methoxycarbonylmethoxybenzo-[b]thiophene-2-carboxylic acid methyl ester (24 mg, 0.06 mmol) was converted to 6-(3-benzylureido)-3-carboxymethoxybenzo[b]thiophene-2-carboxylic acid (19 mg, 83%), following the procedure in the second step of Scheme 20 of Example 57.

¹H NMR (400 MHz, DMSO-D6) δ ppm 4.32 (d, J=5.81 Hz, 2H) 4.96 (s, 2H) 6.79 (t, J=5.81 Hz, 1H) 7.21-7.38 (m, 6H) 7.82 (d, J=8.84 Hz, 1H) 8.14 (d, J=1.52 Hz, 1H) 8.97 (s, 1H).

HRMS (ESI) calcd for C₁₉H₁₆N₂O₆S 401.08019; found 401.07987.

### EXAMPLE 76

### 3-Carboxymethoxy-7-chloro-benzo[b]thiophene-2-carboxylic acid

The first step of Scheme 27: 3-Chloro-2-nitrobenzoic acid methyl ester (1.98 g, 9.2 mmol) was converted to 7-cloro-3-hydroxybenzo[b]thiophene-2-carboxylic acid methyl ester (1.12 g, 50%) after recrystallization from ethanol, following the procedure in the second step of Scheme 24 of Example 63.

¹H NMR (400 MHz, CHLOROFORM-D) δ ppm 3.98 (s, 3H) 7.38 (t, J=7.83 Hz, 1H) 7.49-7.54 (m, 1H) 7.87 (dd, J=8.08, 1.01 Hz, 1H) 10.09 (s, 1H).

The second step of Scheme 27: 7-Chloro-3-hydroxy-benzo[b]thiophene-2-carboxylic acid methyl ester was converted to 7-chloro-3-methoxycarbonylmethoxy-benzo[b]thiophene-2-carboxylic acid methyl ester (1.34 g, 95%), following the procedure in the fourth step of Scheme 21 of Example 58.

¹H NMR (400 MHz, DMSO-D6) δ ppm 3.69 (s, 3H) 3.88 (s, 3H) 5.12 (s, 2H) 7.57 (t, J=7.83 Hz, 1H) 7.75 (dd, J=7.71, 0.88 Hz, 1H) 8.01 (dd, J=8.08, 0.76 Hz, 1H).

The third step of Scheme 27: 7-Chloro-3-methoxycarbonylmethoxy-benzo[b]thiophene-2-carboxylic acid methyl ester was converted to 3-carboxymethoxy-7-chloro-benzo[b]thiophene-2-carboxylic acid (65 mg, 71%), following the procedure in the second step of Scheme 20 of Example 57.

¹H NMR (400 MHz, DMSO-D6) δ ppm 5.02 (s, 2H) 7.53 (t, J=7.83 Hz, 1H) 7.70 (d, J=6.82 Hz, 1H) 7.98 (dd, J=8.08, 1.01 Hz, 1H).

HRMS (ESI) calcd for C₁₁H₆ClO₅S 284.96299; found 284.96231.

### EXAMPLE 77

### 3-Carboxymethoxy-7-methyl-benzo[b]thiophene-2-carboxylic acid

The first and second steps of Scheme 27: 3-Methyl-2-nitro-benzoic acid methyl ester (3.2 g, 17 mmol) was converted to crude 3-hydroxy-7-methyl-benzo[b]thiophene-2-carboxylic acid methyl ester which was subsequently converted to 3-methoxycarbonylmethoxy-7-methylbenzo[b]thiophene-2-carboxylic acid methyl ester (1.56 g, 32% 2 steps), following the procedure in the second step of Scheme 24 of Example 63 and the procedure in the fourth step of Scheme 21 of Example 58.

¹H NMR (400 MHz, CHLOROFORM-D) δ ppm 2.51 (s, 3H) 3.80 (s, 3H) 3.92 (s, 3H) 5.02 (s, 2H) 7.30 (d, J=7.07 Hz, 1H) 7.28-7.32 (m, 1H) 7.91 (d, J=8.08 Hz, 1H)

The third step of Scheme 27: 3-Methoxycarbonylmethoxy-7-methylbenzo-[b]thiophene-2-carboxylic acid methyl ester (100 mg, 0.34 mmol) was converted to 3-carboxymethoxy-7-methyl-benzo[b]thiophene-2-carboxylic acid (81 mg, 90%), following the procedure in the second step of Scheme 20 of Example 57.

¹H NMR (400 MHz, DMSO-D6) δ ppm 2.45 (s, 3H) 4.68 (s, 2H) 7.30 (d, J=7.07 Hz, 1H) 7.33-7.39 (m, 1H) 7.72 (d, J=7.83 Hz, 1H).

HRMS (ESI) calcd for C₁₂H₉O₅S 265.017; found 265.017.

### EXAMPLE 78

### 3-Carboxymethoxy-6-(2-oxo-2-piperidin-1-yl-ethyl)-benzo[b]thiophene-2-carboxylic acid

The first step of Scheme 28: A solution of 3-methoxycarbonylmethoxybenzo-[b]thiophene-2,6-dicarboxylic acid 2-methyl ester (1.0 g, 3 mmol) in thionyl chloride (25 mL) was heated at reflux for 2 h and then concentrated in vacuo. The crude acid chloride was dissolved in THF (30 mL), and to this was added tetrakis(triphenylphosphine)palladium (165 mg, 5 mol %) followed by dropwise addition of tributyltin hydride (0.68 mL, 1.2 eq) over a 10 min period. After stirring at room temperature for 1 h, the solution was absorbed onto silica and flash chromatographed (100% CH₂Cl₂ followed by 30% ethyl acetate/hexanes) to provide 6-formyl-3-methoxycarbonylmethoxybenzo[b]thiophene-2-carboxylic acid methyl ester (800 mg, 84%) as a pale-yellow solid.

¹H NMR (400 MHz, CHLOROFORM-D) δ ppm 3.79 (s, 3H) 3.95 (s, 3H) 5.07 (s, 2H) 7.93 (dd, J=8.46, 1.39 Hz, 1H) 8.23 (d, J=8.59 Hz, 1H) 8.26 (s, 1H) 10.13 (s, 1H).

The second step of Scheme 28: A solution of 6-formyl-3-methoxycarbonylmethoxy-benzo[b]thiophene-2-carboxylic acid methyl ester (800 mg, 0.26 mmol), carbon tetrabromide (1.29 g, 1.5 eq), and triphenylphosphine (2.0 g, 3 eq) in dichloromethane (30 mL) was stirred at 0° C. for 15 min. The solution was diluted with dichloromethane, washed with aqueous sodium bicarbonate and brine, dried over magnesium sulfate, filtered and concentrated in vacuo. Flash chromatography (15% ethyl acetate/hexanes) provided 6-(2,2-dibromovinyl)-3-methoxycarbonylmethoxybenzo[b]thiophene-2-carboxylic acid methyl ester (890 mg, 71%).

¹H NMR (400 MHz, CHLOROFORM-D) δ ppm 3.80 (s, 3H) 3.92 (s, 3H) 5.03 (s, 2H) 7.53 (dd, J=8.84, 1.26 Hz, 1H) 7.58 (s, 1H) 7.95-7.96 (m, 1H) 8.06 (d, J=8.08 Hz, 1H).

The third step of Scheme 28: According to the procedure of Shen and Kunzer (Org. Lett. 2002, 4, 1315-1317), a solution of 6-(2,2-dibromovinyl)-3-methoxycarbonylmethoxy-benzo[b]thiophene-2-carboxylic acid methyl ester (100 mg, 0.2 mmol), piperidine (100 μL, 5 eq), and water (100 μL) in dimethylformamide (1 mL) was heated at 80° C. for 3 h. The cooled solution was diluted with ethyl acetate, washed with water and brine, dried, filtered, evaporated, and flash chromatographed (50-100% ethyl acetate/hexanes) to provide 3-methoxycarbonylmethoxy-6-(2-oxo-2-piperidin-1-ylethyl)benzo[b]thiophene-2-carboxylic acid methyl ester (38 mg, 45%) as a yellow residue.

¹H NMR (400 MHz, CHLOROFORM-D) δ ppm 1.20-1.60 (m, 6H) 3.40 (m, 2H) 3.59 (m, 2H) 3.80 (s, 3H) 3.84 (s, 2H) 3.90 (s, 3H) 5.01 (s, 2H) 7.32 (dd, J=8.34, 1.52 Hz, 1H) 7.63 (s, 1H) 8.01 (d, J=8.34 Hz, 1H).

The fourth step of Scheme 28: 3-Methoxycarbonylmethoxy-6-(2-oxo-2-piperidin-1-ylethyl)benzo[b]thiophene-2-carboxylic acid methyl ester (38 mg, 0.01 mmol) was converted to 3-carboxymethoxy-6-(2-oxo-2-piperidin-1-yl-ethyl)-benzo[b]thiophene-2-carboxylic acid (29 mg, 82%), following the procedure in the second step of Scheme 20 of Example 57.

¹H NMR (400 MHz, CHLOROFORM-D) δ ppm 1.20-1.60 (m, 6H) 3.57 (m, 2H) 3.66 (m, 2H) 3.88 (s, 2H) 4.87 (s, 2H) 7.28 (dd, J=8.34, 1.52 Hz, 1H) 7.58 (s, 1H) 7.73 (d, J=8.34 Hz, 1H).

### EXAMPLE 79

### 3-Ethoxycarbonylmethoxy-6-methyl-thieno[3,2-c]pyridine-2-carboxylic acid methyl ester

The first step of Scheme 29: The aryl chloride (640 mg, 1.9 mmol) was shaken under a hydrogen atmosphere at 45 psi in the presence of 10% Pd/C (0.5 g) in MeOH and EtOAc. The mixture was filtered and the solvent was removed by rotary evaporation. Purification was achieved by silica column chromatography eluting with a gradient from 10% to 75% EtOAc in hexanes. The des-chloro compound was isolated as a yellow solid (134 mg, 23%).

¹H NMR (400 MHz, CHLOROFORM-D) δ ppm 1.27 (t, J=7.07 Hz, 3H) 2.68 (s, 3H) 3.91 (s, 3H) 4.24 (q, J=7.07 Hz, 2H) 5.09 (s, 2H) 7.49 (s, 1H) 9.22 (s, 1H).

### EXAMPLE 80

### 3-Carboxymethoxy-6-methyl-thieno[3,2-c]pyridine-2-carboxylic acid

The second step of Scheme 29: Lithium hydroxide (56 mg) was added to 3-ethoxycarbonylmethoxy-6-methyl-thieno[3,2-c]pyridine-2-carboxylic acid methyl ester (92 mg, 0.3 mmol) in 2 mL THF and 3 mL water and stirred at room temperature overnight. Following the work up procedure in the fifth step of Scheme 13 of Example 24, 3-carboxymethoxy-6-methyl-thieno[3,2-c]pyridine-2-carboxylic acid (28 mg, 35%) was obtained as a white solid.

¹H NMR (400 MHz, DMSO-D6) δ ppm 2.58 (s, 3H) 5.04 (s, 1H) 7.84 (s, 1H) 9.07 (s, 1H).

### EXAMPLE 81

### 3-Carboxymethoxy-5-isobutylamino-thieno[2,3-b]pyridine-2-carboxylic acid

5-Isobutylamino-3-methoxycarbonylmethoxy-thieno[2,3-b]pyridine-2-carboxylic acid methyl ester was prepared according to the procedure in the seventh step of Scheme 4 of Example 8, except that the crude product was purified by flash chromatography using CH₂CL₂/EtOAc (0 to 3% gradient). 5-Isobutylamino-3-methoxycarbonylmethoxy-thieno[2,3-b]pyridine-2-carboxylic acid methyl ester was obtained in 26% yield as a yellow solid.

¹H NMR (400 MHz, CHLOROFORM-D) δ ppm 1.03 (d, J=6.57 Hz, 6H) 1.27 (m, 1H) 3.01 (dd, J=6.32, 4.29 Hz, 2H) 3.80 (s, 3H) 3.90 (s, 3H) 5.00 (s, 2H) 7.37 (d, J=2.78 Hz, 1H) 8.21 (d, J=2.78 Hz, 1H).

5-Isobutylamino-3-methoxycarbonylmethoxy-thieno[2,3-b]pyridine-2-carboxylic acid methyl ester was hydrolyzed following the procedure in the eighth step of Scheme 4 of Example 6 to give 3-carboxymethoxy-5-isobutylamino-thieno[2,3-b]pyridine-2-carboxylic acid in 79% yield as a yellow solid.

¹H NMR (400 MHz, DMSO-D6) δ ppm 0.97 (d, J=6.57 Hz, 6H) 1.90 (m, 1H) 2.88 (d, J=6.82 Hz, 2H) 4.92 (s, 2H) 6.24 (s, 1H) 7.25 (d, J=2.78 Hz, 1H) 8.28 (d, J=2.78 Hz, 1H).

ESI-MS: m/e=325 [M+H]⁺.

### EXAMPLE 82

### 6-Bromo-3-carboxymethoxy-thieno[3,2-b]thiophene-2-carboxylic acid

The first step of Scheme 30: To a solution of 4-bromo-3-hydroxy-thiophene-2-carboxylic acid methyl ester (2.37 g, 10 mmol) in DCM (20 mL) was added TEA (2.09 mL, 15 mmol), DMAP (61 mg, 0.5 mmol) and Tf₂O (2.02 mL, 12 mmol) at 0° C. The reaction mixture was stirred at room temperature for 2 hours, washed with aq. NaHCO₃, and dried over MgSO₄. The crude product was purified on a CombiFlash column eluted with hexanes/EtOAc to give the desired product, 4-bromo-3-trifluoromethanesulfonyloxy-thiophene-2-carboxylic acid methyl ester (3.69 g, 100%).

¹H NMR (400 MHz, CHLOROFORM-D) 8 ppm 3.95 (s, 3H) 7.55 (s, 1H).

The second step of Scheme 30: To a solution of 4-bromo-3-trifluoromethanesulfonyloxy-thiophene-2-carboxylic acid methyl ester (3.55 g, 9.62 mmol) in DMF (55 mL) was added methyl thioglycolate (1.055 mL, 11.8 mmol) and DBU (3.16 mL, 20.2 mmol) at −78° C. The temperature was allowed to rise to room temperature, and the reaction mixture was stirred at room temperature overnight. To this was added ethyl bromoacetate (2.22 mL, 20 mmol) and K₂CO₃ (2.76 g, 20 mmol). The reaction mixture was stirred for additional 6 hours, then diluted with EtOAc and washed with aq. NH₄Cl. The crude product was purified on a CombiFlash column eluted with hexanes/EtOAc to give the desired product, 6-bromo-3-ethoxycarbonylmethoxy-thieno[3,2-b]thiophene-2-carboxylic acid methyl ester (820 mg, 23% overall).

¹H NMR (400 MHz, CHLOROFORM-D) δ ppm 1.28 (t, J=7.20 Hz, 3H) 3.90 (s, 3H) 4.25 (q, J=7.16 Hz, 2H) 5.01 (s, 2H) 7.47 (s, 1H).

Hydrolysis: To a solution of 6-bromo-3-ethoxycarbonylmethoxy-thieno[3,2-b]thiophene-2-carboxylic acid methyl ester (60 mg, 0.158 mmol) in THF (2 mL) and water (2 mL) was added 2.0 M aq. LiOH (0.4 mL, 0.792 mmol) at room temperature. After stirring for 4 hours, the mixture was concentrated and acidified with 10% aq. HCl. The precipitate was collected by filtration to give 6-bromo-3-carboxymethoxy-thieno[3,2-b]thiophene-2-carboxylic acid as a white solid (32 mg, 60%).

¹H NMR (400 MHz, DMSO-D6) δ ppm 4.82 (s, 2H) 7.91 (s, 1H) 13.26 (s, 1H).

### EXAMPLE 83

### 3-Carboxmethoxy-6-(3-cyclohexylamino-phenyl)-thieno[3,2-b]thiophene-2-carboxylic acid

The third step of Scheme 30: 6-Bromo-3-ethoxycarbonylmethoxy-thieno[3,2-b]thiophene-2-carboxylic acid methyl ester (417 mg, 1.1 mmol), 3-aminophenylboronic acid (255.7 mg, 1.65 mmol), Pd(PPh₃)₄ (125.9 mg, 0.11 mmol) and KF (255.21 mg, 4.4 mmol) were mixed and purged with N₂ in a high pressure tube. THF (10 mL) was added and the tube was sealed. The reaction mixture was stirred at 80° C. for 4 hours, diluted with EtOAc and filtered through a pad of Celite. The crude product was purified on a CombiFlash column eluted with hexanes/EtOAc to give the desired product, 6-(3-amino-phenyl)-3-ethoxycarbonylmethoxy-thieno[3,2-b]thiophene-2-carboxylic acid methyl ester (230 mg, 54%) as a light yellow solid.

The fourth step of Scheme 30: To a solution of 6-(3-amino-phenyl)-3-ethoxycarbonylmethoxy-thieno[3,2-b]thiophene-2-carboxylic acid methyl ester (150 mg, 0.418 mmol) in DCE (9 mL) was added cyclohexanone (65 μL, 0.627 mmol), HOAc (36 μL, 0.627 mmol) and NaBH(OAc)₃ (198.4 mg, 0.936 mmol). The resultant mixture was stirred at room temperature overnight before directly loaded to a CombiFlash column, eluted with hexanes/EtOAc to give the desired product, 6-(3-cyclohexylamino-phenyl)-3-ethoxycarbonylmethoxy-thieno[3,2-b]thiophene-2-carboxylic acid methyl ester (165 mg, 83%).

¹H NMR (400 MHz, CHLOROFORM-D) δ ppm 1.12-1.33 (m, 6H) 1.34-1.49 (m, 2H) 1.63-1.71 (m, 1H) 1.71-1.84 (m, 2H) 2.05-2.16 (m, 2H) 3.27-3.40 (m, 1H) 3.89 (s, 3H) 4.27 (q, J=7.16 Hz, 2H) 5.05 (s, 2H) 6.59 (dd, J=8.08, 1.52 Hz, 1H) 6.88 (t, J=1.89 Hz, 1H) 6.93-7.01 (m, 1H) 7.23 (t, J=7.83 Hz, 1H) 7.61 (s, 1H).

The fifth step of Scheme 30: 3-Carboxymethoxy-6-(3-cyclohexylamino-phenyl)-thieno[3,2-b]thiophene-2-carboxylic acid, was prepared from 6-(3-cyclohexylamino-phenyl)-3-ethoxycarbonylmethoxy-thieno[3,2-b]thiophene-2-carboxylic acid methyl ester (38 mg, 0.08 mmol) according to procedures of the hydrolysis step of Example 82 as a white solid (31 mg, 90%).

¹H NMR (400 MHz, DMSO-D6) δ ppm 1.08-1.28 (m, 3H) 1.27-1.44 (m, 2H) 1.54-1.67 (m, 1H) 1.67-1.82 (m, 2H) 1.88-2.03 (m, 2H) 3.17-3.35 (m, 1H) 5.07 (s, 2H) 6.54-6.76 (m, 1H) 6.89-7.03 (m, 1H) 7.14-7.31 (m, 1H) 8.13 (s, 1H).

LCMS m/e: 431.87 [M]⁺.

### EXAMPLE 84

### 6-(3-Acetylamino-phenyl)-3-carboxymethoxy-thieno[3,2-b]thiophene-2-carboxylic acid

Hydrolysis: 6-(3-acetylamino-phenyl)-3-carboxymethoxy-thieno[3,2-b]thiophene-2-carboxylic acid, was prepared from 6-(3-acetylamino-phenyl)-3-ethoxycarbonylmethoxy-thieno[3,2-b]thiophene-2-carboxylic acid methyl ester (21 mg, 0.048 mmol) according to procedures of the hydrolysis step of Example 82 as a white solid (16.5 mg, 87%).

¹H NMR (400 MHz, DMSO-D6) 8 ppm 2.09 (s, 3H) 5.07 (s, 2H) 7.40-7.47 (m, 1H) 7.57-7.66 (m, 1H) 8.01-8.08 (m, 1H) 8.20 (s, 1H) 10.16 (s, 1H).

### EXAMPLE 85

### 3-Carboxymethoxy-6-[3-(cyclohexyl-methoxycarbonyl-amino]-phenyl)-thieno[3,2-b]thiophene-2-carboxylic acid

The fifth step of Scheme 30: To a solution of 6-(3-cyclohexylamino-phenyl)-3-ethoxycarbonylmethoxy-thieno[3,2-b]thiophene-2-carboxylic acid methyl ester (40 mg, 0.1 mmol) in DCM (1 mL) and pyridine (1 mL) was added methyl chloroformate (23.2 μL, 0.3 mmol) and DMAP (cat.) at −78° C. The reaction mixture was then allowed stir at room temperature overnight, then directly loaded to a CombiFlash column, eluted with hexanes/EtOAc to give the desired compound, 6-[3-(cyclohexyl-methoxycarbonyl-amino)-phenyl]-3-ethoxycarbonylmethoxy-thieno[3,2-b]thiophene-2-carboxylic acid methyl ester (36 mg, 68%) as a light yellow oil.

¹H NMR (400 MHz, CHLOROFORM-D) 8 ppm 1.04-1.39 (m, 8H) 1.70 (d, J=13.39 Hz, 2H) 1.87 (d, J=10.86 Hz, 2H) 3.59 (s, 3H) 3.83 (s, 3H) 4.07-4.17 (m, 1H) 4.20 (q, J=7.07 Hz, 2H) 4.99 (s, 2H) 6.98-7.06 (m, 1H) 7.35 (t, J=1.89 Hz, 1H) 7.39 (t, J=7.83 Hz, 1H) 7.53-7.60 (m, 1H) 7.62 (s, 1H).

Hydrolysis: 3-carboxymethoxy-6-[3-(cyclohexyl-methoxycarbonyl-amino)-phenyl]-thieno[3,2-b]thiophene-2-carboxylic acid, was prepared from 6-[3-(cyclohexyl-methoxycarbonyl-amino)-phenyl]-3-ethoxycarbonylmethoxy-thieno[3,2-b]thiophene-2-carboxylic acid methyl ester (31 mg, 0.058 mmol) according to procedures of the hydrolysis step of Example 82 as a white solid (22 mg, 76%).

LCMS m/e: 489.88 [M]⁺.

### EXAMPLE 86

### 3-Carboxymethoxy-6-{3-[cyclohexyl-(3-methyl-butyryl)-amino]-phenyl}-thieno[3,2-b]thiophene-2-carboxylic acid

3-Carboxymethoxy-6-{3-[cyclohexyl-(3-methyl-butyryl)-amino]-phenyl}-thieno[3,2-b]thiophene-2-carboxylic acid was prepared from 6-{3-[cyclohexyl-(3-methyl-butyryl)-amino]-phenyl}-3-ethoxycarbonylmethoxy-thieno[3,2-b]thiophene-2-carboxylic acid methyl ester (34 mg, 0.061 mmol) according to the procedures of the fifth step of Scheme 30, then the hydrolysis step of Example 82 as a white solid (29.7 mg, 94%).

H NMR (400 MHz, MeOD) δ ppm 0.85 (d, J=6.57 Hz, 6H) 0.90-1.26 (m, 5H) 1.34-1.53 (m, 2H) 1.61 (d, J=12.63 Hz, 1H) 1.80 (d, J=11.12 Hz, 2H) 1.91-1.98 (m, 2H) 2.02-2.15 (m, 1H) 4.48-4.68 (m, 1H) 5.10 (s, 2H) 7.21 (d, J=7.83 Hz, 1H) 7.53 (d, J=1.52 Hz, 1H) 7.62 (t, J=7.83 Hz, 1H) 7.84 (d, J=7.83 Hz, 1H) 8.08 (s, 1H).

LCMS m/e: 515.89 [M]+.

### EXAMPLE 87

### 3-Carboxymethoxy-6-[3-(1-cyclohexyl-3-isopropyl-ureido)-phenyl]-thieno[3, 2-b]thiophene-2-carboxylic acid

3-Carboxymethoxy-6-[3-(1-cyclohexyl-3-isopropyl-ureido)-phenyl]-thieno[3,2-b]thiophene-2-carboxylic acid was prepared from 6-[3-(1-cyclohexyl-3-ethyl-ureido)-phenyl]-3-ethoxycarbonylmethoxy-thieno[3,2-b]thiophene-2-carboxylic acid methyl ester (15 mg, 0.028 mmol) according to the procedures of the fifth step of Scheme 30, then the hydrolysis step of Example 82 as a white solid (7 mg, 49%).

¹H NMR (400 MHz, DMSO-D6) δ ppm 0.80-1.02 (m, 3H) 0.97 (d, J=6.57 Hz, 6H) 1.19-1.39 (m, 2H) 1.45-1.57 (m, 1H) 1.68 (d, J=13.90 Hz, 2H) 1.80 (d, J=10.11 Hz, 2H) 3.71-3.87 (m, 1H) 4.12-4.34 (m, 1H) 4.78 (d, J=7.83 Hz, 1H) 7.17 (d, J=7.83 Hz, 1H) 7.42-7.50 (m, 1H) 7.59 (t, J=7.83 Hz, 1H) 7.77 (d, J=8.59 Hz, 1H) 8.35 (s, 1H).

LCMS m/e: 516.94 [M]⁺.

### EXAMPLE 88

### 3-(Carboxymethoxy)thieno[3,2-b]pyridine-2-carboxylic acid

Step 1 of Scheme 31: A solution of 3-mercaptopicolinic acid (750 mg, 3.9 mmol) and hydrochloric acid (3 drops) in methanol (100 mL) was heated at reflux for 48 h. The cooled solution was neutralized with aqueous sodium hydroxide and sodium bicarbonate and evapotated to provide crude methyl 3-mercaptopicolinate.

¹H NMR (400 MHz, CHLOROFORM-D) δ ppm 4.02-4.03 (m, 3H) 7.29 (dd, J=8.08, 4.55 Hz, 1H) 7.69 (dd, J=8.34, 1.52 Hz, 1H) 8.50 (dd, J=4.29, 1.52 Hz, 1H)

Step 2 of Scheme 31: A solution of methyl 3-mercaptopicolinate 149 mg, 0.88 mmol), ethyl bromoacetate (367 mg, 2.5 eq), and potassium carbonate (520 mg) in DMF were heated at 90° C. for 48 h. The cooled solution was acidified with aqueous hydrochloric acid, diluted with water (20 mL), and extracted with ethyl acetate (3×30 mL). The combined organic layers were washed with saturated aqueous sodium chloride, dried with magnesium sulfate, filtered, evaporated, and flash chromatographed (10-75% ethyl acete/hexanes) to provide ethyl 3-(2-ethoxy-2-oxoethoxy)thieno[3,2-b]pyridine-2-carboxylate (66 mg) as a red solid. ¹H NMR (400 MHz, CHLOROFORM-D) δ ppm 1.24 (t, J=7.20 Hz, 3H) 1.41 (t, J=7.07 Hz, 3H) 4.23 (q, J=7.07 Hz, 2H) 4.41 (q, J=7.07 Hz, 2H) 5.47 (s, 2H) 8.08 (dd, J=8.34, 1.52 Hz, 1H) 8.66 (dd, J=4.55, 1.52 Hz, 1H)

Step 3 of Scheme 31: A solution of ethyl 3-(2-ethoxy-2-oxoethoxy)thieno[3,2-b]pyridine-2-carboxylate (55 mg, 0.18 mmol) and lithium hydroxide hydrate (37 mg, 5 eq) in tetrahydrofuran (2 mL) and water (2 mL) was stirred at room temperature for 18 hours. The solution was evaporated, acidified to pH 4, and cooled to 0° C. The resulting precipitate was collected by filtration and dried under vacuum to provide 3-(carboxymethoxy)thieno[3,2-b]pyridine-2-carboxylic acid (4 mg) as an off-white solid.

1H NMR (400 MHz, DMSO-D6) δ ppm 5.23-5.35 (m, 2H) 7.50 (dd, J=8.34, 4.55 Hz, 1H) 8.46 (dd, J=8.34, 1.52 Hz, 1H) 8.69 (dd, J=4.55, 1.52 Hz, 1H).

### EXAMPLE 89

### 3-(Carboxymethoxy)-6-(trifluoromethyl)thieno[3,2-b]pyridine-2-carboxylic acid

Step 1 of Scheme 32: A solution of methyl 3-chloro-5-(trifluoromethyl)picolinate (454 mg, 1.9 mmol), methyl thioglycolate (186 μL, 1.05 eq), sodium tert-butoxide (210 mg) in DMF was stirred at 40° C. for 24 h. Additional sodium tert-butoxide (210 mg) was added and the reaction was heated to 65° C. for 4 h. The cooled solution was diluted with water (30 mL), acidified with aqueous hydrochloric acid to pH 6. The resulting precipitate was collected by filtration to provide methyl 3-hydroxy-6-(trifluoromethyl)thieno[3,2-b]pyridine-2-carboxylate (323 mg, 61%). ¹H NMR (400 MHz, CHLOROFORM-D) δ ppm 4.03 (s, 3H) 8.40 (s, 1H) 9.01 (s, 1H).

Step 2 of Scheme 32: A solution of methyl 3-hydroxy-6-(trifluoromethyl)thieno[3,2-b]pyridine-2-carboxylate (190 mg, 0.72 mmol), tert-butyl bromoacetate (159 μL, 1.5 eq), and sodium tert-butoxide (183 mg, 2.5 eq) in DMF (10 mL) were heated at 60° C. for 16 h. The cooled reaction solution was acidified with aqueous hydrochloric acid, diluted with water, and washed with ethyl acetate (3×20 mL). The combined organic layers were dried, filtered, evaporated and flash chromatographed (ethyl acetate/hexanes 1:4) to provide methyl 3-(2-tert-butoxy-2-oxoethoxy)-6-(trifluoromethyl)thieno[3,2-b]pyridine-2-carboxylate (77 mg, 27%) as a yellow film. ¹H NMR (400 MHz, CHLOROFORM-D) δ ppm 1.43 (s, 9H) 3.96 (s, 3H) 5.42 (s, 2H) 8.36 (d, J=1.26 Hz, 1H) 8.86 (s, 1H).

Step 3 of Scheme 32: A solution of methyl 3-(2-tert-butoxy-2-oxoethoxy)-6-(trifluoromethyl)thieno[3,2-b]pyridine-2-carboxylate (77 mg, 0.2 mmol) and lithium hydroxide hydrate (41 mg, 5 eq) in tetrahydrofuran (5 mL) and water (5 mL) was stirred at room temperature for 18 hours. The solution was acidified, diluted with water (20 mL), and washed with ethyl acetate. The combined organic layers were dried, filtered, evaporated to provide 3-(carboxymethoxy)-6-(trifluoromethyl)thieno[3,2-b]pyridine-2-carboxylic acid (24 mg, 38%) as a light yellow solid. ¹H NMR (400 MHz, DMSO-D6) δ ppm 4.98 (s, 2H) 8.99 (d, J=1.26 Hz, 1H) 9.03 (d, J=1.52 Hz, 1H).

### EXAMPLE 90

### 3-(Carboxymethoxy)thieno[3,2-b]thiophene-2-carboxylic acid

Step 1 of Scheme 33: A solution of methyl 3-chlorothiophene-2-carboxylate (2.75g, 15.6 mmol) methyl thioglycolate (1.42 mL, 1 eq), and potassium carbonate (4.74g, 2 eq) in DMF (60 mL) was heated at 60° C. for 18 h. The cooled solution was diluted with water (100 mL) and washed with ethyl acetate (3×50 mL). The combined organic layers were dried over magnesium sulfate, filtered, evaporated, and flash chromatographed (2%-35% ethyl acetate/hexanes) to provide methyl 3-(2-methoxy-2-oxoethylthio)thiophene-2-carboxylate (81 mg). ¹H NMR (400 MHz, CHLOROFORM-D) δ ppm 3.75 (s, 3H) 3.76 (s, 2H) 3.89 (s, 3H) 7.09 (d, J=5.31 Hz, 1H) 7.26 (s, 1H) 7.51 (d, J=5.31 Hz, 1H).

Step 2 of Scheme 33: A solution of methyl 3-(2-methoxy-2-oxoethylthio)thiophene-2-carboxylate (240 mg, 0.97 mmol) and sodium tert-butoxide (230 mg, 2.5 eq) in DMF (7 mL) was heated at 60° C. for 18 h. The cooled solution was acidified with aqueous hydrochloric acid and washed with ethyl acetate (3×20 mL). The combined organic layers were dried over magnesium sulfate, filtered and evaporated to provide methyl 3-hydroxythieno[3,2-b]thiophene-2-carboxylate (163 mg, 76%) as a red solid. ¹H NMR (400 MHz, CHLOROFORM-D) δ ppm 3.93 (s, 3H) 7.21 (d, J=5.05 Hz, 1H) 7.63 (d, J=5.31 Hz, 1H).

Step 3 of Scheme 33: A solution of methyl 3-hydroxythieno[3,2-b]thiophene-2-carboxylate (140 mg, 0.85 mmol) ethyl bromoacetate (100 μL, 1.1 eq), and sodium tert-butoxide (75 mg, 0.9 eq) in DMF (3 mL) was heated at 40° C. for 2 h. The cooled solution was acidified with aqueous hydrochloric acid and washed with ethyl acetate. The combined organic layers were dried over magnesium sulfate, filtered, and evaporated to provide methyl 3-(2-ethoxy-2-oxoethoxy)thieno[3,2-b]thiophene-2-carboxylate (195 mg, 64%) as a pale-brown liquid. ¹H NMR (400 MHz, CHLOROFORM-D) δ ppm 1.28 (t, J=7.07 Hz, 3H) 3.89 (s, 3H) 4.26 (q, J=7.07 Hz, 2H) 5.02-5.04 (m, 2H) 7.19 (d, J=5.31 Hz, 1H) 7.58 (d, J=5.31 Hz, 1H).

Step 4 of Scheme 33: A solution of methyl 3-(2-ethoxy-2-oxoethoxy)thieno[3,2-b]thiophene-2-carboxylate (71 mg, 0.18 mmol) and lithium hydroxide hydrate (37 mg, 5 eq) in tetrahydrofuran (2 mL) and water (2 mL) was heated at 40° C. for 4 h. The cooled solution was evaporated, and the resulting aqueous mixture was acidified with hydrochloric acid. The resulting yellow precipitate, 3-(carboxymethoxy)thieno[3,2-b]thiophene-2-carboxylic acid (60 mg), was collected by filtration. ¹H NMR (400 MHz, DMSO-D6) δ ppm 4.54 (s, 2H) 7.45 (d, J=5.05 Hz, 1H) 7.85 (d, J=5.31 Hz, 1H).

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A compound of formula (I), 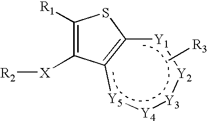
wherein R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O; where one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;
each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —N⁺RC(O)R₅, —N⁺RC(O)NR₅R₆, —N⁺RC(O)OR₅, —N₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₁₂alkyl; where each R₇, R₈, and R₉ is optionally substituted with one or more halogen, oxo, CN, OCF₃, OH, NH₂, or NO₂;
or a salt thereof;
with the proviso that when R₃ is H, the ring system is 1-benzothiophene, R₁ is C(O)OCH₃, and X is —OCH₂—, then R₂ is not C(O)OCH₃;
when R₃ is H, the ring system is 1-benzothiophene, R₁ is C(O)OH, and X is —OCH₂—, then R₂ is not C(O)OH;
when R₃ is H, the ring system is thieno[2,3-b]pyridine, R₁ is isopropyl ester, and X is —OCH₂—, then R₂ is not C₁₋₃alkyl ester;
when R₃ is H, the ring system is thieno[2,3-b]pyridine, R₁ is C(O)OC₁₋₄alkyl, and X is —OCH₂— or —OCH(CH₃)—, then R₂ is not CN;
when R₃ is H, the ring system is thieno[2,3-b]pyridine, R₁ is isopropyl ester, and X is —SCH₂CH₂—, then R₂ is not CN; or
when R₃ is H, the ring system is thieno[2,3-b]pyridine, R₁ is isopropyl ester, and X is —SCH₂—, then R₂ is not isopropyl ester.

2. The compound of claim 1, wherein R₁ is C(O)OH.

3. The compound of claim 1, wherein R₁ is C(O)OCH₃.

4. The compound of claim 1, wherein R₁ is C(O)NH₂.

5. The compound of claim 1, wherein R₁ is C(O)NHCH₃.

6. The compound of claim 1, wherein R₁ is CN.

7. The compound of claim 1, wherein R₁ is a 5-membered heterocycle.

8. The compound of claim 1, wherein X is —O—C₁₋₃alkylene-.

9. The compound of claim 1, wherein X is —OCH₂—.

10. The compound of claim 1, wherein X is —OCHF—.

11. The compound of claim 1, wherein R₂ is C(O)OH.

12. The compound of claim 1, wherein R₂ is C(O)OCH₃.

13. The compound of claim 1, wherein R₂ is C(O)OC₂₋₄alkane.

14. The compound of claim 1, wherein X is —OCH₂— and R₂ is C(O)OH.

15. The compound of claim 1, wherein R₂ is C(O)NH₂.

16. The compound of claim 1, wherein R₂ is CN.

17. The compound of claim 1, wherein Y₅ is absent and each Y₁, Y₂, Y₃, and Y₄ is CR₃.

18. The compound of claim 1, wherein Y₅ is absent and where one of Y₁, Y₂, Y₃, or Y₄ is N and the remaining Y₁, Y₂, Y₃, or Y₄ are each CR₃.

19. The compound of claim 1, wherein X is —OCH₂— and Y₅ is absent and each Y₁, Y₂, Y₃, and Y₄ is CR₃.

20. The compound of claim 1, wherein X is —OCH₂—; Y₅ is absent and each Y₁, Y₂, Y₃, and Y₄ is CR₃; R₁ is C(O)OH; and R₂ is C(O)OH.

21. The compound of claim 1, wherein X is —OCH₂—, Y₅ is absent, and where one of Y₁, Y₂, Y₃, or Y₄ is N and the remaining Y₁, Y₂, Y₃, or Y₄ are each CR₃.

22. The compound of claim 1, wherein X is —OCH₂—; Y₅ is absent, and where one of Y₁, Y₂, Y₃, or Y₄ is N and the remaining Y₁, Y₂, Y₃, or Y₄ are each CR₃; R₁ is C(O)OH; and R₂ is C(O)OH.

23. The compound of claim 1, wherein R₃ is a halogen.

24. The compound of claim 1, wherein R₃ is an optionally substituted aryl.

25. A pharmaceutical composition comprising a compound of formula (I), or a pharmaceutically acceptable salt or prodrug thereof, and a pharmaceutically acceptable excipient or carrier, the compound of formula (I) being: 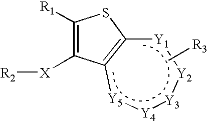
wherein R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O; where one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;
each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₉, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₁₋₁₂alkyl, C₁₋₁₂alkenyl, C₁₋₁₂alkynyl, C₃₋₁₂cycloalkyl, aryl, or arylC₁₋₁₂alkyl; where each R₇, R₈, and R₉ is optionally substituted with one or more halogen, oxo, CN, OCF₃, OH, NH₂, or NO₂.

26. A method of treating a PTPase-mediated disorder or condition comprising administering to a mammal a therapeutically effective amount of a compound of formula (I) or a pharmaceutically acceptable salt or prodrug thereof, the compound of formula (I) being: 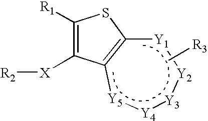
wherein R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O; where one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q; each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₂₋₁₂alkyl, C₂₋₁₂alkenyl, C₂₋₁₂alkynyl, C₃₋₁₂cycloalkyl, aryl, or arylC₁₋₂alkyl; where each R₇, R₈, and R₉ is optionally substituted with one or more halogen, oxo, CN, OCF₃, OH, NH₂, or NO₂.

27. The method of claim 26, wherein the PTPase is PTP1B.

28. The method of claim 26, wherein the disorder or condition is selected from type I diabetes, type II diabetes, obesity, cancer, autoimmune disease, allergic disorder, acute inflammation, chronic inflammation, metabolic syndrome, and osteoporosis.

29. A method of inhibiting a PTPase activity in a sample comprising contacting the sample with an effective amount of a compound of formula (I) or a pharmaceutically acceptable salt or prodrug thereof, the compound of formula (I) being: 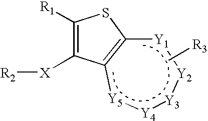
wherein R₁ is C(O)OR₇, 5- to 6-membered heterocycle, H, halogen, CN, or C(O)NR₇R₈;
R₂ is C(O)ZR₄ or CN;
Z is —O— or —NR₅—;
X is —O—C₁₋₃alkylene-, —NR₈—C₁₋₃alkylene-, —S—C₁₋₃alkylene-, —SO—C₁₋₃alkylene-, —SO₂—C₁₋₃alkylene-, —C₁₋₄alkylene-, —C₂₋₄alkenylene-, —C₂₋₄alkynylene-; where any of the alkylene, alkenylene and alkynylene groups is optionally substituted with one or more halogen, oxo, HN═, CN, OCF₃, OH, NH₂, NO₂, R₄, or Q;
each Y₁, Y₂, Y₃, Y₄, and Y₅ is, independently, CR₃, N, S, or O; where one or two of Y₁, Y₂, Y₃, Y₄, and Y₅ can be absent;
each R₃ is, independently, H, aryl, 5- to 8-membered heterocyclyl, C₁₋₆alkyl, C₂₋₆alkenyl, C₂₋₆alkynyl, halogen, CN, OCF₃, OH, NH₂, NO₂, or Q; where any of the aryl, heterocyclic, alkyl, alkenyl or alkynyl groups is optionally substituted with one or more halogen, oxo, CN, OCF₃, OH, NH₂, NO₂, N₃, R₄, or Q;
each Q is, independently, —OC(O)NR₄R₅, —OR₄, —OC(O)R₄, —COOR₄, —C(O)NR₄R₅, —C(O)R₄, —C(═N—OH)R₄, —NR₄R₅, —N⁺R₄R₅R₆, —NR₄C(O)R₅, —NR₄C(O)NR₅R₆, —NR₄C(O)OR₅, —NR₄S(O)₂R₅, —SR₄, —S(O)R₄, —S(O)₂R₄, or —S(O)₂NR₄R₅;
each R₄, R₅, and R₆ is, independently, H, C₁₋₁₆alkyl, C₂₋₁₂alkenyl, C₂₋₁₂alkynyl, C₃₋₈cycloalkyl, cycloalkylC₁₋₆alkyl, 5- to 8-membered heterocycle, heterocyclicC₁₋₆alkyl, aryl, arylC₁₋₆alkyl, arylC₂₋₆alkenyl, or arylC₂₋₆alkynyl; where each R₄, R₅, and R₆ is optionally substituted with one or more C₁₋₆alkyl, C₂₋₆alkenyl, C₂₋₆alkynyl, halogen, oxo, CN, OCF₃, OH, NH₂, NO₂, N₃, —OC(O)NR₇R₈, —OR₇, —OC(O)R₇, —COOR₇, —C(O)NR₇R₈, —C(O)R₇, —NR₇R₈, —N⁺R₇R₈R₉, —NR₇C(O)R₈, —NR₇C(O)NR₈R₉, —NR₇C(O)OR₈, —NR₇S(O)₂R₈, —SR₇, —S(O)R₇, —S(O)₂R₇, or —S(O)₂NR₇R₈;
each R₇, R₈, and R₉ is, independently, H, C₁₋₁₂alkyl, C₂₋₁₂alkenyl, C₂₋₁₂alkynyl, C₃₋₁₂cycloalkyl, aryl, or arylC₁₋₂alkyl; where each R₇, R₈, and R₉ is optionally substituted with one or more halogen, oxo, CN, OCF₃, OH, NH₂, or NO₂.

30. The method of claim 29, wherein the PTPase is PTP1B.
